SEQUENCE LISTING (185 sequences)

SEQ ID NO: 1 - protein, 56 aa, Homo sapiens
  ref <308>: DATABASE ACCESSION NUMBER: AAM71141
  ref <309>: DATABASE ENTRY DATE: 2002-10-07
  ref <313>: RELEVANT RESIDUES IN SEQ ID NO: (1)..(56)
TGTSHLVKCAEKEKTFCVNGGECFMVKDLSNPSRYLCKCQPGFTGARCTENVPMKV

SEQ ID NO: 2 - protein, 56 aa, Homo sapiens
  ref <308>: DATABASE ACCESSION NUMBER: AAM71136
  ref <309>: DATABASE ENTRY DATE: 2002-10-07
  ref <313>: RELEVANT RESIDUES IN SEQ ID NO: (1)..(56)
TGTSHLVKCAEKEKTFCVNGGECFMVKDLSNPSRYLCKCPNEFTGDRCQNYVMASF

SEQ ID NO: 3 - protein, 53 aa, Homo sapiens
  ref <308>: DATABASE ACCESSION NUMBER: NP_004874
  ref <309>: DATABASE ENTRY DATE: 2005-11-27
  ref <313>: RELEVANT RESIDUES IN SEQ ID NO: (1)..(53)
SWSGHARKCNETAKSYCVNGGVCYYIEGINQLSCKCPNGFFGQRCLEKLPLRL

SEQ ID NO: 4 - protein, 53 aa, Homo sapiens
  ref <308>: DATABASE ACCESSION NUMBER: NP_053586
  ref <309>: DATABASE ENTRY DATE: 2005-11-27
  ref <313>: RELEVANT RESIDUES IN SEQ ID NO: (1)..(53)
SWSGHARKCNETAKSYCVNGGVCYYIEGINQLSCKCPVGYTGDRCQQFAMVNF

SEQ ID NO: 5 - protein, 55 aa, Homo sapiens
  ref <308>: DATABASE ACCESSION NUMBER: P56975
  ref <309>: DATABASE ENTRY DATE: 2006-02-07
  ref <313>: RELEVANT RESIDUES IN SEQ ID NO: (1)..(55)
ERSEHFKPCRDKDLAYCLNDGECFVIETLTGSHKHCRCKEGYQGVRCDQFLPKTD

SEQ ID NO: 6 - protein, 53 aa, Homo sapiens
  ref <308>: DATABASE ACCESSION NUMBER: NP_612640
  ref <309>: DATABASE ENTRY DATE: 2005-10-17
  ref <313>: RELEVANT RESIDUES IN SEQ ID NO: (1)..(53)
MPTDHEEPCGPSHKSFCLNGGLCYVIPTIPSPFCRCVENYTGARCEEVFLPGS

SEQ ID NO: 7 - protein, 53 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(53)
SVRNSDSECPLSHDGYCLHDGVCMYIEALDKYACNCVVGYIGERCQYRDLKWW

SEQ ID NO: 8 - protein, 52 aa, Homo sapiens
  ref <308>: DATABASE ACCESSION NUMBER: P01135
  ref <309>: DATABASE ENTRY DATE: 2006-02-07
  ref <313>: RELEVANT RESIDUES IN SEQ ID NO: (1)..(52)
AVVSHFNDCPDSHTQFCFHGTCRFLVQEDKPACVCHSGYVGARCEHADLLAV

SEQ ID NO: 9 - protein, 52 aa, Homo sapiens
  ref <308>: DATABASE ACCESSION NUMBER: P35070
  ref <309>: DATABASE ENTRY DATE: 2006-02-07
  ref <313>: RELEVANT RESIDUES IN SEQ ID NO: (1)..(52)
KRKGHFSRCPKQYKHYCIKGRCRFVVAEQTPSCVCDEGYIGARCERVDLFYL

SEQ ID NO: 10 - protein, 52 aa, Homo sapiens
  ref <308>: DATABASE ACCESSION NUMBER: AAA51781
  ref <309>: DATABASE ENTRY DATE: 1994-10-31
  ref <313>: RELEVANT RESIDUES IN SEQ ID NO: (1)..(52)
RNRKKKNPCNAEFQNFCIHGECKYIEHLEAVTCKCQQEYFGERCGEKSMKTH

SEQ ID NO: 11 - protein, 52 aa, Homo sapiens
  ref <308>: DATABASE ACCESSION NUMBER: AAH33097
  ref <309>: DATABASE ENTRY DATE: 2005-01-18
  ref <313>: RELEVANT RESIDUES IN SEQ ID NO: (1)..(52)
GLGKKRDPCLRKYKDFCIHGECKYVKELRAPSCICHPGYHGERCHGLSLPVE

SEQ ID NO: 12 - protein, 52 aa, Homo sapiens
  ref <308>: DATABASE ACCESSION NUMBER: NP_001423
  ref <309>: DATABASE ENTRY DATE: 2006-01-29
  ref <313>: RELEVANT RESIDUES IN SEQ ID NO: (1)..(52)
VAQVSITKCSSDMNGYCLHGQCIYLVDMSQNYCRCEVGYTGVRCEHFFLTVH

SEQ ID NO: 13 - protein, 52 aa, Mus musculus
  ref <308>: DATABASE ACCESSION NUMBER: CAC39435
  ref <309>: DATABASE ENTRY DATE: 2005-04-15
  ref <313>: RELEVANT RESIDUES IN SEQ ID NO: (1)..(52)
VALKFSHPCLEDHNSYCINGACAFHHELKQAICRCFTGYTGQRCEHLTLTSY

SEQ ID NO: 14 - protein, 57 aa, Homo sapiens
  ref <308>: DATABASE ACCESSION NUMBER: AM71141
  ref <309>: DATABASE ENTRY DATE: 2002-10-07
  ref <313>: RELEVANT RESIDUES IN SEQ ID NO: (1)..(57)
TGTSHLVKCAEKEKTFCVNGGECFMVKDLSNPSRYLCKCQPGFTGARCTENVPMKVQ

SEQ ID NO: 15 - protein, 57 aa, Homo sapiens
  ref <308>: DATABASE ACCESSION NUMBER: AAM71136
  ref <309>: DATABASE ENTRY DATE: 2002-10-07
  ref <313>: RELEVANT RESIDUES IN SEQ ID NO: (1)..(57)
TGTSHLVKCAEKEKTFCVNGGECFMVKDLSNPSRYLCKCPNEFTGDRCQNYVMASFY

SEQ ID NO: 16 - protein, 54 aa, Homo sapiens
  ref <308>: DATABASE ACCESSION NUMBER: NP_004874
  ref <309>: DATABASE ENTRY DATE: 2005-11-07
  ref <313>: RELEVANT RESIDUES IN SEQ ID NO: (1)..(54)
SWSGHARKCNETAKSYCVNGGVCYYIEGINQLSCKCPNGFFGQRCLEKLPLRLY

SEQ ID NO: 17 - protein, 54 aa, Homo sapiens
  ref <308>: DATABASE ACCESSION NUMBER: NP_053586
  ref <309>: DATABASE ENTRY DATE: 2005-11-27
  ref <313>: RELEVANT RESIDUES IN SEQ ID NO: (1)..(54)
SWSGHARKCNETAKSYCVNGGVCYYIEGINQLSCKCPVGYTGDRCQQFAMVNFY

SEQ ID NO: 18 - protein, 55 aa, Homo sapiens; identical to SEQ ID NO: 5
  ref <308>: DATABASE ACCESSION NUMBER: P56975
  ref <309>: DATABASE ENTRY DATE: 2006-02-07
  ref <313>: RELEVANT RESIDUES IN SEQ ID NO: (1)..(55)

SEQ ID NO: 19 - protein, 54 aa, Homo sapiens
  ref <308>: DATABASE ACCESSION NUMBER: NP_612640
  ref <309>: DATABASE ENTRY DATE: 2005-10-17
  ref <313>: RELEVANT RESIDUES IN SEQ ID NO: (1)..(54)
MPTDHEEPCGPSHKSFCLNGGLCYVIPTIPSPFCRCVENYTGARCEEVFLPGSS

SEQ ID NO: 20 - protein, 54 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(54)
SVRNSDSECPLSHDGYCLHDGVCMYIEALDKYACNCVVGYIGERCQYRDLKWWE

SEQ ID NO: 21 - protein, 53 aa, Homo sapiens
  ref <308>: DATABASE ACCESSION NUMBER: P01135
  ref <309>: DATABASE ENTRY DATE: 2006-02-07
  ref <313>: RELEVANT RESIDUES IN SEQ ID NO: (1)..(53)
AVVSHFNDCPDSHTQFCFHGTCRFLVQEDKPACVCHSGYVGARCEHADLLAVV

SEQ ID NO: 22 - protein, 53 aa, Homo sapiens
  ref <308>: DATABASE ACCESSION NUMBER: P35070
  ref <309>: DATABASE ENTRY DATE: 2006-02-07
  ref <313>: RELEVANT RESIDUES IN SEQ ID NO: (1)..(53)
KRKGHFSRCPKQYKHYCIKGRCRFVVAEQTPSCVCDEGYIGARCERVDLFYLR

SEQ ID NO: 23 - protein, 53 aa, Homo sapiens
  ref <308>: DATABASE ACCESSION NUMBER: AAA51781
  ref <309>: DATABASE ENTRY DATE: 1994-10-31
  ref <313>: RELEVANT RESIDUES IN SEQ ID NO: (1)..(53)
RNRKKKNPCNAEFQNFCIHGECKYIEHLEAVTCKCQQEYFGERCGEKSMKTHS

SEQ ID NO: 24 - protein, 53 aa, Homo sapiens
  ref <308>: DATABASE ACCESSION NUMBER: AAH33097
  ref <309>: DATABASE ENTRY DATE: 2005-01-18
  ref <313>: RELEVANT RESIDUES IN SEQ ID NO: (1)..(53)
GLGKKRDPCLRKYKDFCIHGECKYVKELRAPSCICHPGYHGERCHGLSLPVEN

SEQ ID NO: 25 - protein, 53 aa, Homo sapiens
  ref <308>: DATABASE ACCESSION NUMBER: NP_001423
  ref <309>: DATABASE ENTRY DATE: 2006-01-29
  ref <313>: RELEVANT RESIDUES IN SEQ ID NO: (1)..(53)
VAQVSITKCSSDMNGYCLHGQCIYLVDMSQNYCRCEVGYTGVRCEHFFLTVHQ

SEQ ID NO: 26 - protein, 53 aa, Mus musculus
  ref <308>: DATABASE ACCESSION NUMBER: CAC39435
  ref <309>: DATABASE ENTRY DATE: 2005-04-15
  ref <313>: RELEVANT RESIDUES IN SEQ ID NO: (1)..(53)
VALKFSHPCLEDHNSYCINGACAFHHELKQAICRCFTGYTGQRCEHLTLTSYA

SEQ ID NO: 27 - protein, 37 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(37)
CKLRKGNCSSTVCGQDLQSHLCMCAEGYALSRDRKYC

SEQ ID NO: 28 - protein, 36 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(36)
CAFWNHGCTLGCKNTPGSYYCTCPVGFVLLPDGKRC

SEQ ID NO: 29 - protein, 36 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(36)
CPRNVSECSHDCVLTSEGPLCFCPEGSVLERDGKTC

SEQ ID NO: 30 - protein, 38 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(38)
CSSPDNGGCSQLCVPLSPVSWECDCFPGYDLQLDEKSC

SEQ ID NO: 31 - protein, 36 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(36)
CLYQNGGCEHICKKRLGTAWCSCREGFMKASDGKTC

SEQ ID NO: 32 - protein, 34 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(34)
CAPVGCSMYARCISEGEDATCQCLKGFAGDGKLC

SEQ ID NO: 33 - protein, 37 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(37)
CEMGVPVCPPASSKCINTEGGYVCRCSEGYQGDGIHC

SEQ ID NO: 34 - protein, 36 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(36)
CQLGVHSCGENASCTNTEGGYTCMCAGRLSEPGLIC

SEQ ID NO: 35 - protein, 37 aa, Homo sapiens
  ref <308>: DATABASE ACCESSION NUMBER: NP_001954
  ref <309>: DATABASE ENTRY DATE: 2006-01-08
  ref <313>: RELEVANT RESIDUES IN SEQ ID NO: (1)..(37)
CPLSHDGYCLHDGVCMYIEALDKYACNCVVGYIGERC

SEQ ID NO: 36 - protein, 34 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(34)
CSQPGETCLNGGKCEAANGTEACVCGGAFVGPRC

SEQ ID NO: 37 - protein, 36 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(36)
CLSTPCKNAGTCHVVDRRGVADYACSCALGFSGPLC

SEQ ID NO: 38 - protein, 33 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(33)
CLTNPCRNGGTCDLLTLTEYKCRCPPGWSGKSC

SEQ ID NO: 39 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CASNPCANGGQCLPFEASYICHCPPSFHGPTC

SEQ ID NO: 40 - protein, 34 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(34)
CGQKPRLCRHGGTCHNEVGSYRCVCRATHTGPNC

SEQ ID NO: 41 - protein, 33 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(33)
CSPSPCQNGGTCRPTGDVTHECACLPGFTGQNC

SEQ ID NO: 42 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CPGNNCKNGGACVDGVNTYNCPCPPEWTGQYC

SEQ ID NO: 43 - protein, 34 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(34)
CQLMPNACQNGGTCHNTHGGYNCVCVNGWTGEDC

SEQ ID NO: 44 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CASAACFHGATCHDRVASFYCECPHGRTGLLC

SEQ ID NO: 45 - protein, 34 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(34)
CISNPCNEGSNCDTNPVNGKAICTCPSGYTGPAC

SEQ ID NO: 46 - protein, 34 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(34)
CSLGANPCEHAGKCINTLGSFECQCLQGYTGPRC

SEQ ID NO: 47 - protein, 32 aa, Mus musculus
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CVSNPCQNDATCLDQIGEFQCMCMPGYEGVHC

SEQ ID NO: 48 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CASSPCLHNGRCLDKINEFQCECPTGFTGHLC

SEQ ID NO: 49 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CASTPCKNGAKCLDGPNTYTCVCTEGYTGTHC

SEQ ID NO: 50 - protein, 31 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(31)
CDPDPCHYGSCKDGVATFTCLCRPGYTGHHC

SEQ ID NO: 51 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CSSQPCRLRGTCQDPDNAYLCFCLKGTTGPNC

SEQ ID NO: 52 - protein, 31 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(31)
CASSPCDSGTCLDKIDGYECACEPGYTGSMC

SEQ ID NO: 53 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CAGNPCHNGGTCEDGINGFTCRCPEGYHDPTC

SEQ ID NO: 54 - protein, 31 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(31)
CNSNPCVHGACRDSLNGYKCDCDPGWSGTNC

SEQ ID NO: 55 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CESNPCVNGGTCKDMTSGIVCTCREGFSGPNC

SEQ ID NO: 56 - protein, 32 aa, Homo sapiens
CASNPCLNKGTCIDDVAGYKCNCLLPYTGATC

SEQ ID NO: 57 - protein, 35 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(35)
CAPSPCRNGGECRQSEDYESFSCVCPTAGAKGQTC

SEQ ID NO: 58 - protein, 32 aa, Homo sapiens
  misc_feature (18)..(18): X = undefined amino acid
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CVLSPCRHGASCQNTHGXYRCHCQAGYSGRNC

SEQ ID NO: 59 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CRPNPCHNGGSCTDGINTAFCDCLPGFRGTFC

SEQ ID NO: 60 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CASDPCRNGANCTDCVDSYTCTCPAGFSGIHC

SEQ ID NO: 61 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CTESSCFNGGTCVDGINSFTCLCPPGFTGSYC

SEQ ID NO: 62 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CDSRPCLLGGTCQDGRGLHRCTCPQGYTGPNC

SEQ ID NO: 63 - protein, 32 aa, Homo sapiens
  ref <308>: DATABASE ACCESSION NUMBER: AAG33848
  ref <309>: DATABASE ENTRY DATE: 2001-11-22
  ref <313>: RELEVANT RESIDUES IN SEQ ID NO: (1)..(32)
CDSSPCKNGGKCWQTHTQYRCECPSGWTGLYC

SEQ ID NO: 64 - protein, 42 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(42)
CEVAAQRQGVDVARLCQHGGLCVDAGNTHHCRCQAGYTGSYC

SEQ ID NO: 65 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CSPSPCQNGATCTDYLGGYSCKCVAGYHGVNC

SEQ ID NO: 66 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CLSHPCQNGGTCLDLPNTYKCSCPRGTQGVHC

SEQ ID NO: 67 - protein, 40 aa, Mus musculus
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(40)
CNPPVDPVSRSPKCFNNGTCVDQVGGYSCTCPPGFVGERC

SEQ ID NO: 68 - protein, 34 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(34)
CLSNPCDARGTQNCVQRVNDFHCECRAGHTGRRC

SEQ ID NO: 69 - protein, 35 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(35)
CKGKPCKNGGTCAVASNTARGFICKCPAGFEGATC

SEQ ID NO: 70 - protein, 32 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(32)
CGSLRCLNGGTCISGPRSPTCLCLGPFTGPEC

SEQ ID NO: 71 - protein, 35 aa, Homo sapiens
  ref <308>: DATABASE ACCESSION NUMBER: AAG33848
  ref <309>: DATABASE ENTRY DATE: 2000-11-22
  ref <313>: RELEVANT RESIDUES IN SEQ ID NO: (1)..(35)
CLGGNPCYNQGTCEPTSESPFYRCLCPAKFNGLLC

SEQ ID NO: 72 - protein, 36 aa, Homo sapiens
CPDSHTQFCFHGTCRFLVQEDKPACVCHSGYVGARC

SEQ ID NO: 73 - protein, 38 aa, Homo sapiens
  ref <308>: DATABASE ACCESSION NUMBER: NT_007995.11
  ref <309>: DATABASE ENTRY DATE: 2003-01-04
  ref <313>: RELEVANT RESIDUES IN SEQ ID NO: (1)..(38)
TGTSHLVKCAEKEKTFCVNGGECFMVKDLSNPSRYLCK

SEQ ID NO: 74 - protein, 35 aa, Homo sapiens
  ref <308>: DATABASE ACCESSION NUMBER: NT_029289
  ref <309>: DATABASE ENTRY DATE: 2004-08-19
  ref <313>: RELEVANT RESIDUES IN SEQ ID NO: (1)..(35)
SWSGHARKCNETAKSYCVNGGVCYYIEGINQLSCK

SEQ ID NO: 75 - protein, 37 aa, Homo sapiens
  ref <308>: DATABASE ACCESSION NUMBER: NT_033890
  ref <309>: DATABASE ENTRY DATE: 2003-04-10
  ref <313>: RELEVANT RESIDUES IN SEQ ID NO: (1)..(37)
ERSEHFKPCRDKDLAYCLNDGECFVIETLTGSHKHCR

SEQ ID NO: 76 - protein, 35 aa, Homo sapiens
  ref <308>: DATABASE ACCESSION NUMBER: NT_024654
  ref <309>: DATABASE ENTRY DATE: 2003-01-05
  ref <313>: RELEVANT RESIDUES IN SEQ ID NO: (1)..(35)
MPTDHEEPCGPSHKSFCLNGGLCYVIPTIPSPFCR

SEQ ID NO: 77 - protein, 35 aa, Homo sapiens
  ref <308>: DATABASE ACCESSION NUMBER: NT_028147
  ref <309>: DATABASE ENTRY DATE: 2002-08-01
  ref <313>: RELEVANT RESIDUES IN SEQ ID NO: (1)..(35)
SVRNSDSECPLSHDGYCLHDGVCMYIEALDKYACK

SEQ ID NO: 78 - protein, 34 aa, Homo sapiens
  ref <308>: DATABASE ACCESSION NUMBER: NT_22184.9
  ref <309>: DATABASE ENTRY DATE: 2003-01-03
  ref <313>: RELEVANT RESIDUES IN SEQ ID NO: (1)..(34)
AVVSHFNDCPDSHTQFCFHGTCRFLVQEDKPACV

SEQ ID NO: 79 - protein, 34 aa, Homo sapiens
  ref <308>: DATABASE ACCESSION NUMBER: NT_034698
  ref <309>: DATABASE ENTRY DATE: 2002-08-01
  ref <313>: RELEVANT RESIDUES IN SEQ ID NO: (1)..(34)
KRKGHFSRCPKQYKHYCIKGRCRFVVAEQTPSCV

SEQ ID NO: 80 - protein, 34 aa, Homo sapiens
  ref <308>: DATABASE ACCESSION NUMBER: NT_006216.12
  ref <309>: DATABASE ENTRY DATE: 2003-01-03
  ref <313>: RELEVANT RESIDUES IN SEQ ID NO: (1)..(34)
RNRKKKNPCNAEFQNFCIHGECKYIEHLEAVTCK

SEQ ID NO: 81 - protein, 34 aa, Homo sapiens
  ref <308>: DATABASE ACCESSION NUMBER: NT_034777.1
  ref <309>: DATABASE ENTRY DATE: 2002-08-01
  ref <313>: RELEVANT RESIDUES IN SEQ ID NO: (1)..(34)
GLGKKRDPCLRKYKDFCIHGECKYVKELRAPSCM

SEQ ID NO: 82 - protein, 34 aa, Homo sapiens
  ref <308>: DATABASE ACCESSION NUMBER: NT_006216.11
  ref <309>: DATABASE ENTRY DATE: 2003-01-03
  ref <313>: RELEVANT RESIDUES IN SEQ ID NO: (1)..(34)
VAQVSITKCSSDMNGYCLHGQCIYLVDMSQNYCR

SEQ ID NO: 83 - protein, 34 aa, Mus musculus
  ref <308>: DATABASE ACCESSION NUMBER: NT_039307.1
  ref <309>: DATABASE ENTRY DATE: 2003-02-24
  ref <313>: RELEVANT RESIDUES IN SEQ ID NO: (1)..(34)
VALKFSHPCLEDHNSYCINGACAFHHELKQAICR

SEQ ID NO: 84 - protein, 34 aa, Homo sapiens
  ref <308>: DATABASE ACCESSION NUMBER: NT_006216.1
  ref <309>: DATABASE ENTRY DATE: 2001-02-09
  ref <313>: RELEVANT RESIDUES IN SEQ ID NO: (1)..(34)
IALKFSHLCLEDHNSYCINGACAFHHELEKAICR

SEQ ID NO: 85 - protein, 360 aa, Homo sapiens
TARGAGEEFPETCWNSGLARRPGAERRRLPDDGSVSRTVITSPRSGCEGAGQRPGREPPAAGPIDDFPGRQEQPREPGRAPVPGGRTARRVRAALPAGNGRRPRAARAPQRGRSLSPSRDKLFPNPIRALGPNSPAPRAVRVERSVSGEMSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCK

SEQ ID NO: 86 - protein, 43 aa, Homo sapiens
TSTSTTGTSHLVKCAEKEKTFCVNGGECFMVKDLSNPSRYLCK

SEQ ID NO: 87 - protein, 43 aa, Homo sapiens; identical to SEQ ID NO: 86

SEQ ID NO: 88 - protein, 211 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCK

SEQ ID NO: 89 - protein, 211 aa, Homo sapiens; identical to SEQ ID NO: 88

SEQ ID NO: 90 - protein, 211 aa, Mus musculus
MSERKEGRGKGKGKKKDRGSRGKPAPAEGDPSPALPPRLKEMKSQESAAGSKLVLRCETSSEYSSLRFKWFKNGNELNRRNKPQNVKIQKKPGKSELRINKASLADSGEYMCKVISKLGNDSASANITIVESNDLTTGMSASTERPYVSSESPIRISVSTEGANTSSSTSTSTTGTSHLIKCAEKEKTFCVNGGECFMVKDLSNPSRYLCK

SEQ ID NO: 91 - protein, 211 aa, Mus musculus; identical to SEQ ID NO: 90

SEQ ID NO: 92 - protein, 73 aa, Mus musculus
MSASTERPYVSSESPIRISVSTEGANTSSSTSTSTTGTSHLIKCAEKEKTFCVNGGECFMVKDLSNPSRYLCK

SEQ ID NO: 93 - protein, 137 aa, Homo sapiens
  misc_feature (113)..(113): X = undefined amino acid
TRPKLKKMKSQTGQVGEKQSLKCEAAAINPQPSYRWFKDGKELNRSRDIRIKYGNGRKNSRLQFNKVKVEDAGEYVCEAENILGKDTVRGRLYVNSVTTTLSSWSGHAGKCNXTAKSYCVNGGVCYYIEGINQLSCK

SEQ ID NO: 94 - protein, 73 aa, Homo sapiens
SSSSFDVGHEGDDSWGLGIVSVRHWHMSLIPSVSTTLSSWSGHARKCNETAKSYCVNGGVCYYIEGINQLSCK

SEQ ID NO: 95 - protein, 78 aa, Homo sapiens
EINIIIWYYFPSAWRTCFNISSSVGLLLTNSYKFYTTTYSTERSEHFKPCRDKDLAYCLNDGECFVIETLTGSHKHCR

SEQ ID NO: 96 - protein, 42 aa, Homo sapiens
NYLQIKMPTDHEEPCGPSHKSFCLNGGLCYVIPTIPSPFCRK

SEQ ID NO: 97 - protein, 36 aa, Homo sapiens
MPTDHEEPCGPSHKSFCLNGGLCYVIPTIPSPFCRK

SEQ ID NO: 98 - protein, 36 aa, Homo sapiens; identical to SEQ ID NO: 97

SEQ ID NO: 99 - protein, 37 aa, Mus musculus
MPTGNFLSRAALWSQAQVILPQWGDLLCDPYYPQPIL

SEQ ID NO: 100 - protein, 37 aa, Mus musculus; identical to SEQ ID NO: 99

SEQ ID NO: 101 - protein, 25 aa, Homo sapiens
SHKSFCLNGGLCYVIPTIPSPFCRK

SEQ ID NO: 102 - protein, 30 aa, Sus scrofa
EPCGPSHRSFCLNGGICYVIPTIPSPFCRK

SEQ ID NO: 103 - protein, 30 aa, Sus scrofa; identical to SEQ ID NO: 102

SEQ ID NO: 104 - protein, 46 aa, Mus musculus
CLFAPADSPVAAAVVSHFNKCPDSHTQYCFHGTCRFLVQEEKPACV

SEQ ID NO: 105 - protein, 51 aa, Homo sapiens
DLSPASFLSPADPPVAAAVVSHFNDCPDSHTQFCFHGTCRFLVQEDKPACV

SEQ ID NO: 106 - protein, 42 aa, Homo sapiens
VQTEDNPRVAQVSITKCSSDMNGYCLHGQCIYLVDMSQNYCR

SEQ ID NO: 107 - protein, 40 aa, Homo sapiens
QTEDNPRVAQVSITKCSSDMNGYCLHGQCIYLVDMSQNYC

SEQ ID NO: 108 - protein, 42 aa, Mus musculus
VQMEDDPRVAQVQITKCSSDMDGYCLHGQCIYLVDMREKFCR

SEQ ID NO: 109 - protein, 93 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCR

SEQ ID NO: 110 - protein, 93 aa, Homo sapiens; identical to SEQ ID NO: 109

SEQ ID NO: 111 - protein, 180 aa, Homo sapiens
  misc_feature: X = undefined amino acid
  misc_feature (118)..(118): X = undefined amino acid
PGEKATRPKLKKMKSQTGQVGEKQSLKCEAAAGNPQPSYRWFKDGKELNRSRDIRIKYGNGRKNSRLQFNKVKVEDAGEYVCEAENILGKDTVGGRLYVNSVTTTLSSWSGHARKCNXTAKSYCVNGGVCYYIEGINQLSCKAPGLHCLELGTQSHHFPISASPGSSQGSWNQLPQHPLS

SEQ ID NO: 112 - protein, 120 aa, Homo sapiens
  misc_feature (13)..(13): X = undefined amino acid
EAENILGKDTVRXRLYVNSVSTTLSSWSGHARKCNETAKSYCVNGGVCYYIEGINQLSCKAHGLHCLELGTQSHHFPISASPGSSQGSWNQLPQHPLSALGGEGSPGGDAVRTPGPQSCA

SEQ ID NO: 113 - protein, 76 aa, Mus musculus
VRQRRETPSPPIAGSRMARNSTGVVIFASSMAMAVSTTLSSWSGHARKCNETAKSYCVNGGVCYYIEGINQLSCKG

SEQ ID NO: 114 - protein, 167 aa, Danio rerio
KDCASAPKVKPMDSQWLQEGKKLTLKCEAVGNPSPSFNWYKDGSQLRQKKTVKIKTNKKNSKLHISKVRLEDSGNYTCVVENSLGRENATSFVSVQSITTTLSPGSSHARKCNETEKTYCINGGDCYFIHGINQLSCKCPNDYTGERCQTSVMAGFYKAEELYQNEC

SEQ ID NO: 115 - protein, 84 aa, Gallus gallus
AVQSLELLQQTWRLSTLQFEYDRRVACGFHYTTTYSTERSEHFKPCKDKDLAYCLNEGECFVIETLTGSHKHCRSNCPSGVFCW

SEQ ID NO: 116 - protein, 77 aa, Gallus gallus
MRTDHEELCGTSYGSFCLNGGICYMIPTVPSPFCRHLPKAANQASALHKSVFSIFVLHTDTTALPSCHLMPAHFYTQ

SEQ ID NO: 117 - protein, 65 aa, Mus musculus
MPTDHEQPCGPRHRSFCLNGGICIDPYYPHPFCRFYHLFLRHCLLKPFVQLGTLVYPVFLKELFH

SEQ ID NO: 118 - protein, 70 aa, Homo sapiens
DVIAQHKPESENTSDKPKRKKKGGKNGKNRRNRKKKNPCDAEFQNFCIHGECKYIEHLEAVTCNVSRIFP

SEQ ID NO: 119 - protein, 112 aa, Homo sapiens
  misc_feature (2)..(2): X = undefined amino acid
LXATTQSKWKGHSSRCPKQYKHYCIKGRCRFVVAEQTPSCVPLRKRRKRKKKEEEMETLGKDMTPINEDIEETNIAYKAMKLPPGWWQAAKCLAHLKMDRMRLRKTASRHEF

SEQ ID NO: 120 - protein, 119 aa, Mus musculus
KSLTWKSFNFLSLLLPLGSTGTRRILCPLSTPSCSAGLAILHCVVADGNTTRTPETNGSLCGAPGENCTGTTPRQKVKTHFSRCPKQYKHYCIHGRCRFVVDEQTPSCMARLSIYLWRN

SEQ ID NO: 121 - protein, 141 aa, Cercopithecus aethiops (African green monkey)
MKLLPSVVLKLLLAAVLSALVTGESLEQLRRGPAAGTSNPDPSTGSTDQLLRLGGGRDRKVRDLQEADLDLLRVTLSSKPQALATPSKEEHGKRKKKGKGLGKKRDPCLRKYKDFCIHGECKYVKELRAPSCMAAGQKDVT

SEQ ID NO: 122 - protein, 79 aa, Homo sapiens
MTALTEEAAVTVTPPITAQQADNIEGPIALKFSHLCLEDHNSYCINGACAFHHELEKAICRCLKLKSPYNVCSGERRPL

SEQ ID NO: 123 - protein, 96 aa, Homo sapiens
GTREALCYRCFCPLNTAMRALTEEAAVTVTPPITAQQADNIEGPIALKFSHLCLEDHNSYCINGACAFHHELEKAICRCLKLKSPYNVCSGERRPL

SEQ ID NO: 124 - protein, 96 aa, Homo sapiens; identical to SEQ ID NO: 123

SEQ ID NO: 125 - protein, 97 aa, Homo sapiens
LQEMALGVPISVYLLFNAMTALTEEAAVTVTPPITAQQADNIEGPIALKFSHLCLEDHNSYCINGACAFHHELEKAICRCLKLKSPYNVCSGERRPL

SEQ ID NO: 126 - protein, 115 aa, Homo sapiens
KDKRKKVKQLQEMALGVPISVYLLFNAMTALTEEAAVTVTPPITAQQGNWTVNKTEADNIEGPIALKFSHLCLEDHNSYCINGACAFHHELEKAICRCLKLKSPYNVCSGERRPL

SEQ ID NO: 127 - protein, 94 aa, Homo sapiens
MALGVPISVYLLFNAMTALTEEAAVTVTPPITAQQADNIEGPIALKFSHLCLEDHNSYCINGACAFHHELEKAICRCLKLKSPYNVCSGERRPL

SEQ ID NO: 128 - DNA, 117 bp, Homo sapiens
  ref <308>: DATABASE ACCESSION NUMBER: NT_007995.10
  ref <309>: DATABASE ENTRY DATE: 2003-01-04
  ref <313>: RELEVANT RESIDUES IN SEQ ID NO: (1)..(117)
actgggacaagccatcttgtaaaatgtgcggagaaggagaaaactttctgtgtgaatggaggggagtgcttcatggtgaaagacctttcaaacccctcgagatacttgtgcaagtaa

SEQ ID NO: 129 - DNA, 108 bp, Homo sapiens
  ref <308>: DATABASE ACCESSION NUMBER: NT_029289
  ref <309>: DATABASE ENTRY DATE: 2004-08-19
  ref <313>: RELEVANT RESIDUES IN SEQ ID NO: (1)..(108)
tcctggtcggggcacgcccggaagtgcaacgagacagccaagtcctattgcgtcaatggaggcgtctgctactacatcgagggcatcaaccagctctcctgcaagtaa

SEQ ID NO: 130 - DNA, 114 bp, Homo sapiens
  ref <308>: DATABASE ACCESSION NUMBER: NT_0033890.2
  ref <309>: DATABASE ENTRY DATE: 2003-04-10
  ref <313>: RELEVANT RESIDUES IN SEQ ID NO: (1)..(114)
gagcgatccgagcacttcaaaccctgccgagacaaggaccttgcatactgtctcaatgatggcgagtgctttgtgatcgaaaccctgaccggatcccataaacactgtcggtaa

SEQ ID NO: 131 - DNA, 99 bp, Homo sapiens
  ref <308>: DATABASE ACCESSION NUMBER: NT_024654.12
  ref <309>: DATABASE ENTRY DATE: 2003-01-05
  ref <313>: RELEVANT RESIDUES IN SEQ ID NO: (1)..(99)
gatcacgaagagccctgtggtcccagtcacaagtcgttttgcctgaatggggggctttgttatgtgatacctactattcccagcccattttgtaggtga

SEQ ID NO: 132 - DNA, 108 bp, Homo sapiens
  ref <308>: DATABASE ACCESSION NUMBER: NT_028147.9
  ref <309>: DATABASE ENTRY DATE: 2002-08-01
  ref <313>: RELEVANT RESIDUES IN SEQ ID NO: (1)..(108)
tccgtaagaaatagtgactctgaatgtcccctgtcccacgatgggtactgcctccatgatggtgtgtgcatgtatattgaagcattggacaagtatgcatgcaagtaa

SEQ ID NO: 133 - DNA, 105 bp, Homo sapiens
  ref <308>: DATABASE ACCESSION NUMBER: NT_022184.9
  ref <309>: DATABASE ENTRY DATE: 2003-01-03
  ref <313>: RELEVANT RESIDUES IN SEQ ID NO: (1)..(105)
gcagtggtgtcccattttaatgactgcccagattcccacactcagttctgcttccatggaacctgcaggtttttggtgcaggaggacaagccagcatgtgtgtaa

SEQ ID NO: 134 - DNA, 105 bp, Homo sapiens
  ref <308>: DATABASE ACCESSION NUMBER: NT_034698.1
  ref <309>: DATABASE ENTRY DATE: 2002-08-01
  ref <313>: RELEVANT RESIDUES IN SEQ ID NO: (1)..(105)
aagcggaaaggccacttctctaggtgccccaagcaatacaagcattactgcatcaaagggagatgccgcttcgtggtggccgagcagacgccctcctgtgtgtaa

SEQ ID NO: 135 - DNA, 105 bp, Homo sapiens
  ref <308>: DATABASE ACCESSION NUMBER: NT_06216.11
  ref <309>: DATABASE ENTRY DATE: 2003-01-03
  ref <313>: RELEVANT RESIDUES IN SEQ ID NO: (1)..(105)
agaaacagaaagaagaaaaatccatgtaatgcagaatttcaaaatttctgcattcacggagaatgcaaatatatagagcacctggaagcagtaacatgcaagtaa

SEQ ID NO: 136 - DNA, 105 bp, Homo sapiens
  ref <308>: DATABASE ACCESSION NUMBER: NT_034777.1
  ref <309>: DATABASE ENTRY DATE: 2002-08-01
  ref <313>: RELEVANT RESIDUES IN SEQ ID NO: (1)..(105)
gggctagggaagaagagggacccatgtcttcggaaatacaaggacttctgcatccatggagaatgcaaatatgtgaaggagctccgggctccctcctgcatgtaa

SEQ ID NO: 137 - DNA, 105 bp, Homo sapiens
  ref <308>: DATABASE ACCESSION NUMBER: NT_06216.11
  ref <309>: DATABASE ENTRY DATE: 2003-01-03
  ref <313>: RELEVANT RESIDUES IN SEQ ID NO: (1)..(105)
gtggctcaagtgtcaataacaaagtgtagctctgacatgaatggctattgtttgcatggacagtgcatctatctggtggacatgagtcaaaactactgcaggtaa

SEQ ID NO: 138 - DNA, 105 bp, Mus musculus
  ref <308>: DATABASE ACCESSION NUMBER: NT_039307.1
  ref <309>: DATABASE ENTRY DATE: 2003-02-24
  ref <313>: RELEVANT RESIDUES IN SEQ ID NO: (1)..(105)
gtagctctgaagttctctcatccttgtctggaagaccataatagttactgcattaatggagcatgtgcattccaccatgagctgaagcaagccatttgcaggtaa

SEQ ID NO: 139 - DNA, 105 bp, Homo sapiens
  ref <308>: DATABASE ACCESSION NUMBER: NT_06216.12
  ref <309>: DATABASE ENTRY DATE: 2001-02-09
  ref <313>: RELEVANT RESIDUES IN SEQ ID NO: (1)..(105)
atagccttgaagttctcacacctttgcctggaagatcataacagttactgcatcaacggtgcttgtgcattccaccatgagctagagaaagccatctgcaggtaa

SEQ ID NO: 140 - DNA, 1651 bp, Homo sapiens
  ref <308>: DATABASE ACCESSION NUMBER: A81177.1
  ref <309>: DATABASE ENTRY DATE: 2000-01-21
  ref <313>: RELEVANT RESIDUES IN SEQ ID NO: (1)..(1651)
  ref <302>: TITLE: HEREGULIN GAMMA
  ref <308>: DATABASE ACCESSION NUMBER: A81177.1
  ref <309>: DATABASE ENTRY DATE: 2000-01-21
  ref <310>: PATENT DOCUMENT NUMBER: WO9914323
  ref <311>: PATENT FILING DATE: 1997-10-17
  ref <312>: PUBLICATION DATE: 1999-03-25
  ref <313>: RELEVANT RESIDUES IN SEQ ID NO: (1)..(1651)
acggcacgaggagccggcgaggagttccccgaaacttgttggaactccgggctcgcgcggaggccaggagctgagcggcggcggctgccggacgatgggagcgtgagcaggacggtgataacctctccccgatcgggttgcgagggcgccgggcagaggccaggacgcgagccgccagcggcgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccatcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtaagaaaagaaatcctgtgtgtcgcttatgtctataactccttgtttcagatgattctatgtctcatgattgattgttgctttttttccaattttgttgcatcatgttgaataatgctgttttatatgtagagtcttttaaaacattcacaccattcgtcatcactcctctgtcatatgcagttttgttttttgctcttttcaatgtgtgtgaggtgttttttgtttttgtttttgtttttttgccatgttatttatagtgttgctttccttgtgctttccttgtggttttcttggttggttattcagaaaagatgtgcagatatcacagaggcctatagccttttggtatctacttctacatccaatgtatgaattaagctgtaagataatgttgctttcttatcccagtgatcacctgccaaatgaataagacaacaaagagaagcagaagggcaagaagattatttactgacatatatctattacacttgggattgtgcttactgttgcataactattttttaaacggagtttagttttatattgctagtaaaaaaaaaaaaaaaaaa

SEQ ID NO: 141 - DNA, 675 bp, Homo sapiens
  ref <302>: TITLE: HUMAN SCHIZOPHRENIA GENE
  ref <308>: DATABASE ACCESSION NUMBER: AX269478
  ref <309>: DATABASE ENTRY DATE: 2001-11-30
  ref <310>: PATENT DOCUMENT NUMBER: WO0164876
  ref <311>: PATENT FILING DATE: 2001-02-28
  ref <312>: PUBLICATION DATE: 2001-09-07
  ref <313>: RELEVANT RESIDUES IN SEQ ID NO: (1)..(675)
ctacatctacatccaccactgggacaagccatcttgtaaaatgtgcggagaaggagaaaactttctgtgtgaatggaggggagtgcttcatggtgaaagacctttcaaacccctcgagatacttgtgcaagtaagaaaagaaatcctgtgtgtcgcttatgtctataactccttgtttcagatgattctatgtctcatgatgtattgttgctttttttccaattttgttgcatcatgttgaataatgctgttttatatgtagagtgttttaaaacattcacaccattcgtcatcactcctctgtcatatgcagaattgttttttgctcttttcaatgtgtgtgaggtgttttttgtttttgtttttgttttttgccatgttatttatagtgttgctttccttgtggtttttcttgttgttattcagaaaagatgtgcagatatcacagaggcctataacttttggtatctacttctacatccaatgtatgaattaagctgtaagataatgttgctttcttatcccrgtgatcacctgccaaatgaataagacaacaaagagaagcagaagggcagaagattatttactgacatatatctattacacttgggattgtctyactgttgcataactattttttaaacggagtttagttttatattgctagtaaaaaaa

SEQ ID NO: 142 - DNA, 675 bp, Homo sapiens; identical to SEQ ID NO: 141
  ref <302>: TITLE: HUMAN SCHIZOPHRENIA GENE
  ref <308>: DATABASE ACCESSION NUMBER: AX271009.1
  ref <309>: DATABASE ENTRY DATE: 2001-11-30
  ref <310>: PATENT DOCUMENT NUMBER: WO0164877
  ref <311>: PATENT FILING DATE: 2001-02-28
  ref <312>: PUBLICATION DATE: 2001-09-07
  ref <313>: RELEVANT RESIDUES IN SEQ ID NO: (1)..(675)

SEQ ID NO: 143 - DNA, 1651 bp, Homo sapiens; identical to SEQ ID NO: 140
  ref <308>: DATABASE ACCESSION NUMBER: NM_004495.1
  ref <309>: DATABASE ENTRY DATE: 2006-02-12
  ref <313>: RELEVANT RESIDUES IN SEQ ID NO: (1)..(1651)

SEQ ID NO: 144 - DNA, 1651 bp, Homo sapiens; identical to SEQ ID NO: 140
  ref <308>: DATABASE ACCESSION NUMBER: AF026146
  ref <309>: DATABASE ENTRY DATE: 1999-01-05
  ref <313>: RELEVANT RESIDUES IN SEQ ID NO: (1)..(1651)

SEQ ID NO: 145 - DNA, 1590 bp, Mus musculus
  ref <308>: DATABASE ACCESSION NUMBER: NM_178591
  ref <309>: DATABASE ENTRY DATE: 2003-12-22
  ref <313>: RELEVANT RESIDUES IN SEQ ID NO: (1)..(1590)
gactccgggccgcgccggcagcaggagcggaacgcagcgcagcggcggcagctgccaggagatgcgagcatagaccggactgtgagcacctttccctcttcgggctgtaagggagcgagacagccaccggagcgaggccactccagagccggcagcggcaggacccgggacacaagagtagccccgagacacccccagacgtagcgggcgctccaggtgatcgagtccacgccgctccctgcaggcgacaggcgacgcccccgcgcagcccggccactggctcttccctcccgggacaaacttttctgcaagcccttggaccaaacttgtcgcgcgtcaccgtcgcccagccgggtccgcgtagagcgctcatctttagcgagatgtctgagcgcaaagaaggcagaggcaaggggaagggcaagaagaaggaccggggatcccgcgggaagcccgcgcccgccgaaggcgacccgagcccagcattgcctcccagattgaaagagatgaaaagccaggagtcagctgcaggctccaagctcgtgcttcggtgtgaaaccagctctgagtactcctcactcagattcaaatggttcaagaacgggaatgagctgaaccgtaggaataaaccacaaaacgtcaagatacagaagaagccagggaagtcagagcttcgaatcaacaaagcgtccctggctgactctggagaatatatgtgcaaagtgatcagcaagttaggaaacgacagtgcctctgccaacatcaccattgttgagtcaaacgacctcaccactggcatgtcagcctcaactgaaagaccttatgtgtcctcagagtctcccattagaatatcagtttcaacagaaggcgcaaatacttcttcatccacatctacatccacgactgggaccagccatctcataaagtgtgcggagaaggagaaaactttctgtgtgaatggaggcgagtgcttcatggtgaaggacctgtcaaacccctcaagatacttgtgcaagtaagaaatgaattcctctctgtgcctcgtacctgtaacagcttatcccagattgttctgtgtcgccatgaacccctggcttttttttccttactttgttacatcttgttttaaataattctcatttatttgtggagggttttttgaaatatttgcaccatctgccattgcctctgtcatgttcagaattgattttacttttcaaggttttagggtgtttttggttcttgatgggttgagtattttttttgtttggttggttttgggtttttgctgttttgttttgttttttgtttttgttttcttttttgccttcatatatataattttgctttcctcctggtgttccttaatagctactgaaagaagtgtgcaaatattgtagaaagctgtcactttgaatccctacttttttatcccatgtattaattgagccataaggtacataaggtaacttttttttaacctcagtgcttacctgcaaggtgaacaggacaaatagaggttgcaagagagcagaaagttacctgctaaagcatttcttatgctctggattatggtattgccccataatt

SEQ ID NO: 146 - DNA, 1630 bp, Mus musculus
  ref <308>: DATABASE ACCESSION NUMBER: AK051824.1
  ref <309>: DATABASE ENTRY DATE: 1999-01-05
  ref <313>: RELEVANT RESIDUES IN SEQ ID NO: (1)..(1630)
gactccgggccgcgccggcagcaggagcggaacgcagcgcagcggcggcagctgccaggagatgcgagcatagaccggactgtgagcacctttccctcttcgggctgtaagggagcgagacagccaccggagcgaggccactccagagccggcagcggcaggacccgggacacaagagtagccccgagacacccccagacgtagcgggcgctccaggtgatcgagtccacgccgctccctgcaggcgacaggcgacgcccccgcgcagcccggccactggctcttccctcccgggacaaacttttctgcaagcccttggaccaaacttgtcgcgcgtcaccgtcgcccagccgggtccgcgtagagcgctcatctttagcgagatgtctgagcgcaaagaaggcagaggcaaggggaagggcaagaagaaggaccggggatcccgcgggaagcccgcgcccgccgaaggcgacccgagcccagcattgcctcccagattgaaagagatgaaaagccaggagtcagctgcaggctccaagctcgtgcttcggtgtgaaaccagctctgagtactcctcactcagattcaaatggttcaagaacgggaatgagctgaaccgtaggaataaaccacaaaacgtcaagatacagaagaagccagggaagtcagagcttcgaatcaacaaagcgtccctggctgactctggagaatatatgtgcaaagtgatcagcaagttaggaaacgacagtgcctctgccaacatcaccattgttgagtcaaacgacctcaccactggcatgtcagcctcaactgaaagaccttatgtgtcctcagagtctcccattagaatatcagtttcaacagaaggcgcaaatacttcttcatccacatctacatccacgactgggaccagccatctcataaagtgtgcggagaaggagaaaactttctgtgtgaatggaggcgagtgcttcatggtgaaggacctgtcaaacccctcaagatacttgtgcaagtaagaaatgaattcctctctgtgcctcgtacctgtaacagcttatcccagattgttctgtgtcgccatgaacccctggcttttttttccttactttgttacatcttgttttaaataattctcatttatttgtggagggttttttgaaatatttgcaccatctgccattgcctctgtcatgttcagaattgattttacttttcaaggttttagggtgtttttggttcttgatgggttgagtattttttttgtttggttggttttgggtttttgctgttttgttttgttttttgtttttgttttcttttttgccttcatatatataattttgctttcctcctggtgttccttaatagctactgaaagaagtgtgcaaatattgtagaaagctgtcactttgaatccctacttttttatcccatgtattaattgagccataaggtacataaggtaacttttttttaacctcagtgcttacctgcaaggtgaacaggacaaatagaggttgcaagagagcagaaagttacctgctaaagcatttcttatgctctggattatggtattgccccataattagttttcaagacaaattttaagttgccctttctagttact

SEQ ID NO: 147 - DNA, 366 bp, Mus musculus
  ref <308>: DATABASE ACCESSION NUMBER: BY212704.1
  ref <309>: DATABASE ENTRY DATE: 2002-12-10
  ref <313>: RELEVANT RESIDUES IN SEQ ID NO: (1)..(366)
ttcaaggcactgctcgtccttgctcgcactcatttgcccttggatcataggcgatggccccagctcctagcctcctgcactaccccataatcgtctgtcacccttttgttttttgcagagctcacaactggcatgtcagcctcaactgaaagaccctatgtgtcctcagagtctcccattagaatatcagtttcaacagaaggcgcaaatacttcttcatccacatctacatccacgactgggacaagccatctaataaagtgtgcggagaaggagaaaactttctgtgtgaacggaggcgagtgcttcatggtgaaggacctgtcaaacccctcaagatacttgtgcaagtaagaaatgaattcc

SEQ ID NO: 148 - DNA, 412 bp, Homo sapiens
  misc_feature (339)..(339): n = undefined nucleitide
  ref <308>: DATABASE ACCESSION NUMBER: AI041451.1
  ref <309>: DATABASE ENTRY DATE: 1998-08-28
  ref <313>: RELEVANT RESIDUES IN SEQ ID NO: (339)..(339)
cacccggcccaagttgaagaagatgaagagccagacgggacaggtgggtgagaagcaatcgctgaagtgtgaggcagcagcgataaatccccagccttcctaccgttggttcaaggatggcaaggagctcaaccgcagccgagacattcgcatcaaatatggcaacggcagaaagaactcacgactacagttcaacaaggtgaaggtggaggacgctggggagtatgtctgcgaggccgagaacatcctggggaaggacaccgtacgaggccggctttacgtcaacagcgtgacgaccaccctgtcatcctggtcggggcacgccgggaagtgcaacgngacagccaagtcctattgcgtcaatggaggcgtctgctactacatcgagggcatcaaccagctctcctgcaag

SEQ ID NO: 149 - DNA, 350 bp, Homo sapiens
  ref <308>: DATABASE ACCESSION NUMBER: AX406619.1
  ref <309>: DATABASE ENTRY DATE: 2002-06-14
  ref <313>: RELEVANT RESIDUES IN SEQ ID NO: (1)..(350)
ggtcatcttccagttttgacgtggggcatgaaggagatgattcctggggcctagggatagtctcagtgcgtcactggcacatgtctctcataccctcagtgagcaccaccctgtcatcctggtcggggcacgcccggaagtgcaacgagacagccaagtcctattgcgtcaatggaggcgtctgctactacatcgagggcatcaaccagctctcctgcaagtaagtgaccagtaggggtgggcatgggagcaagaacagggtaggagatgctgggtcagaagtggagggctctaggaaaagagggttccaagccactgacaagaggtccccaaggggtgtagacaggaag

SEQ ID NO: 150 - DNA, 629 bp, Homo sapiens
  misc_feature (554)..(554): n = undefined nucleitide
  misc_feature (577)..(577): n = undefined nucleitide
  misc_feature (594)..(594): n = undefined nucleitide
  ref <308>: DATABASE ACCESSION NUMBER: BX495970
  ref <309>: DATABASE ENTRY DATE: 2003-09-04
  ref <313>: RELEVANT RESIDUES IN SEQ ID NO: (1)..(629)
gggagtcaagagatggcagtacttggctgaaggttggtagtgagagatcaatataatcatctggtattattttccttctgcctggaggacttgctttaacatttcaagtagtgtgggtctgctgctgacgaattcatacaaattttatacgacgacatattccacagagcgatccgagcacttcaaaccctgccgagacaaggaccttgcatactgtctcaatgatggcgagtgctttgtgatcgaaaccctgaccggatcccataaacactgtcggtaagccactgaggccactgatggaaagggcaggcccgttgcaaggcgtgggggtggagggtgctggcagcatctggtatgtgtcatatccgggatacacacagtcccaccgtttgaatagcagaattgcgagtcttaatttggaaagggcaaggctgctgcctctttaacagtggaagaagacaaaatggaaacaaagtagttacggtttaagttttacctgaccaagcaaacaaagatttacttttagatctgcaaagttaatggaaataattatntacacactttagaagcgtctgtntatgatgtggagcttangcatatatcctagtactcagaaataatctgttctt

SEQ ID NO: 151 - DNA, 595 bp, Homo sapiens
  misc_feature (205)..(205): n = undefined nucleitide
  ref <308>: DATABASE ACCESSION NUMBER: BE787057.1
  ref <309>: DATABASE ENTRY DATE: 2000-10-20
  ref <313>: RELEVANT RESIDUES IN SEQ ID NO: (1)..(595)
gtgtctgcggtattcaaaaacttttgaaacactgcatgtccaacaaaatttattttttgtgtgaatgtaagtttttattgagggtactgtttttcaaccctactctcttgaccaagaatgaaactatttacaaattaagatgccaacagatcacgaagagccctgtggtcccagtcacaagtcgttttgcctgaatggggggctntgttatgtgatacctactattcccagcccattttgtaggaagtgaactgatgctggcttctctttgtcttattccaagttgggcatgagattttccctgcattagaaggttgttgagacctgaagcctgggaaggtgcgttgaaaactatacaggagctcgttgtgaagaggtttttctcccaggctccagcatccaaactaaaagtaacctgtttgaagcttttgtggcattggcggtcctagtaacacttatcattggagccttctacttcctttgcaggaaaggccactttcagagagccagttcagtccagtatgatatcaacctggtagagacgagcagtaccagtgcccaccacagtcatgaacaacactgaagaaacgtcaaa

SEQ ID NO: 152 - DNA, 545 bp, Homo sapiens
  ref <308>: DATABASE ACCESSION NUMBER: BF061527.1
  ref <309>: DATABASE ENTRY DATE: 2000-10-16
  ref <313>: RELEVANT RESIDUES IN SEQ ID NO: (1)..(545)
taagaaataaaggattagatttttaattcttttacctagtggtgtttcattttctgcctttgtaaaataaaaacaatgatttggttcactttgacgtttcttcagtgttgttcatgactgtggtgggcactggtactgctcgtctctaccaggttgatatcatactggactgaactggctctctgaaagtggcctttcctgcaaaggaagtagaaggctccaatgataagtgttactaggaccgccaatgccacaaaagcttcaaacaggttacttttagtttggatgctggagcctgggagaaaaacctcttcacaacgagctcctgtatagttttcaacgcaccttcccaggcttcaggtctcaacaaccttctaatgcagggaaaatctcatgcccaacttggaataagacaaagagaagccagcatcagttcacttcctacaaaatgggctgggaatagtaggtatcacataacaaagccccccattcaggcaaaacgacttgtgactgggaccacagggctcttcgtgatctgttggcat

SEQ ID NO: 153 - DNA, 715 bp, Homo sapiens
  ref <308>: DATABASE ACCESSION NUMBER: BX095400.1
  ref <309>: DATABASE ENTRY DATE: 2003-02-04
  ref <313>: RELEVANT RESIDUES IN SEQ ID NO: (1)..(715)
gcctgagctgggcagggggcggaggcgggggctcggctgtctccggggctgccacgcagagcgggcttcgtggcgtggatgaagaaactgaggcacagagggattaagtagcctgctcaagatcacacagctagtaaggaaccaagattcaaacttgggcagtgtgattcagagactttaaattcaacgctggtgcctcactgcctcacactaaaagtgaatcagaaaaataaagaaccagcatcaaatttgaagtggccacaaattctattaaagcagaagaaatagtggtgaaccataaaagataaccagtttcctctctattctgcaatttagaggaaaaattttcatccaaggacagatcaggtggtggacctagatgggaaacccaaattataatcaagagatttcttggtactgtttttcaaccctactctcttgaccaagaatgaaactatttacaaattaagatgccaacagatcacgaagagccctgtggtcccagtcacaagtcgttttgcctgaatggggggctttgttatgtgatacctactattcccagcccattttgtaggaagtgaactgatgctggcttctctttgtcttattccaagttgggcatgagattttccctgcattagaaggttgttgagacctgaagcctgggaaggtgcgttgaaaactatacaggagctcgttgtgaagaggtttttct

SEQ ID NO: 154 - DNA, 669 bp, Mus musculus
  ref <308>: DATABASE ACCESSION NUMBER: BB637399
  ref <309>: DATABASE ENTRY DATE: 2001-10-26
  ref <313>: RELEVANT RESIDUES IN SEQ ID NO: (1)..(669)
gagtgttcaaacacttgtgaaacgctgcatgtctagcaaaattttctttttttatgggaatataaatttctgttgaggtgctgattttcaaccttaattcttccatcaagaatgaaactatttaaaaattaagatgccaacaggtaatttcttatcacgagcagccctgtggtcccaggcacaggtcattttgcctcaatggggggatttgttatgtgatccctactatccccagcccattctgtaggaagtgaactgttgctggcttctctttgtcttattccaagttgggtcatgagattttccctgcaccctgggaaggtgcattgaaaattacaccggagcacgctgcgaagaggtttttctcccaagctccagcatcccaagcgaaagtaatctgtcggcagctttcgtggtgctggcggtcctcctcactcttaccatcgcggcgctctgcttcctgtgcagggccgagtggaactgaccctccaggacatatgtgagatgctaaaaggaagactaaagaagtggaagggccaccttcagagggccagttcagtccaatgtgagatcagcctggtggaaacaaacaataccagaacccgtcacagccacagaaaacactggaaacatacatccccagggaagggcatcattacctacaaaggg

SEQ ID NO: 155 - DNA, 614 bp, Mus musculus
  ref <308>: DATABASE ACCESSION NUMBER: BB637505.1
  ref <309>: DATABASE ENTRY DATE: 2001-10-26
  ref <313>: RELEVANT RESIDUES IN SEQ ID NO: (1)..(614)
gagtgttcaaacacttgtgaaacgctgcatgtctagcaaaattttctttttttatgggaatataaatttctgttgaggtgctgattttcaaccttaattcttccatcaagaatgaaactatttaaaaattaagatgccaacaggtaatttcttatcacgagcagccctgtggtcccaggcacaggtcattttgcctcaatggggggatttgttatgtgatccctactatccccagcccattctgtaggaagtgaactgttgctggcttctctttgtcttattccaagttgggtcatgagattttccctgcaccctgggaaggtgcattgaaaattacaccggagcacgctgcgaagaggtttttctcccaagctccagcatcccaagcgaaagtaatctgtcggcagctttcgtggtgctggcggtcctcctcactcttaccatcgcggcgctctgcttcctgtgcaggaagggccaccttcagagggccagttcagtccagtgtgagatcagcctggtagagacaaacaataccagaacccgtcacagccacagagaacactgaagacatacatccccagtgaagggcatcattacctacaaaggcggactgg

SEQ ID NO: 156 - DNA, 513 bp, Homo sapiens
  ref <308>: DATABASE ACCESSION NUMBER: AI743118
  ref <309>: DATABASE ENTRY DATE: 1999-12-20
  ref <313>: RELEVANT RESIDUES IN SEQ ID NO: (1)..(513)
ttaagaaataaaggattagatttttaattcttttacctagtggtgtttcattttctgcctttgtaaaataaaaacaatgatttggttcactttgacgtttcttcagtgttgttcatgactgtggtgggcactggtactgctcgtctctaccaggttgatatcatactggactgaactggctctctgaaagtggcctttcctgcaaaggaagtagaaggctccaatgataagtgttactaggaccgcccatgccacaaaagcttcaaacaggttacttttagtttggatgctggagcctgggagaaaaacctcttcacaacgagctcctgtatagttttcaacgcaccttcccaggcttcaggtctcaacaaccttctaatgcagggaaaatctcatgcccaacttggaataagacaaagagaagccagcatcagttcacttcctacaaaatgggctgggaatagtaggtatcacataacaaagccccccattcaggcaaaacgacttgtgact

SEQ ID NO: 157 - DNA, 243 bp, Sus scrofa
  ref <308>: DATABASE ACCESSION NUMBER: AU059620.1
  ref <309>: DATABASE ENTRY DATE: 1999-04-29
  ref <313>: RELEVANT RESIDUES IN SEQ ID NO: (1)..(243)
aagagccctgtggtcccagtcacaggtcattttgcctgaatggagggatttgttatgtgatacctactattcccagccccttttgtaggaagtgaactgatgctggcttctctttgtcttattccaagttggggcatgagattttgcctgcattagaaggttgttgagacctgaagcctggtaaggtcatgcagaacattgaagaaataccatagtgaactcaaaatcgttgcttctttgtta

SEQ ID NO: 158 - DNA, 300 bp, Sus scrofa
  misc_feature (111)..(275): n = undefined nucleitide
  ref <308>: DATABASE ACCESSION NUMBER: C94578
  ref <309>: DATABASE ENTRY DATE: 1998-06-10
  ref <313>: RELEVANT RESIDUES IN SEQ ID NO: (1)..(300)
aagagccctgtggtcccagtcacaggtcattttgcctgaatggagggatttgttatgtgatacctactattcccagccccttttgtaggaagtgaactgatgctggctttncnttggcctaatnccagnttgggcatgagatttgcctgcattagaanggtgttgaganctgaagcctggtaaaggcatgcagaacattgaagaatacntagtgaactccaaatcggtgcttccttggtacaaaaggcgnaatgnagcccatacggtaaagatcnatgagttaatcctccttggccccaa

SEQ ID NO: 159 - DNA, 2360 bp, Mus musculus
  ref <308>: DATABASE ACCESSION NUMBER: AK089870.1
  ref <309>: DATABASE ENTRY DATE: 2005-09-02
  ref <313>: RELEVANT RESIDUES IN SEQ ID NO: (1)..(2360)
ttgtttgttgttgcatacaccaggctgctggacactgaacttctggcaattctcttgtctctgaccccatctcctggtagaggtgcactggactacagacatgtgccctactgcactggctatttatgtggatttgaactcaggtcatcaggctgtggggcgagtgccttaccctctgaactatcttcccagcccctgttgttggcttgtgtctcatgtgttagggaggttcagtgccctcatggcacttggcagtgctttgtgaggcaccagagagttggaggccaccatggtgtgacatgaccctttgcatgtccttccagctatttctcaggctggatacaaagtgccaggtgcatggaaacttcattatagaggttcaggtacccaggtcaatgttttcctcaggaactctaagtagaaaactaaactctagtcagtttgctattaaaaacagatcccagctcaagcgtcccgggactccttttgtaccctggacatctggttgacagttctcatccttcaacttgctcagccctctgggtctcagatcagtagccagccacatagaagcaaacactcttttaatcgggtacttggccacccccttcctcccctaagacgaggggaatactcacacacatgctggcttctcttcctgcaccaaaaaccggcaggttccatggaagcagtactgagtgtgggaatctgggcacttgttgaagtgagacaccactgcagccgccacgggtgagtctgctggggcaaagagacatcatcagacctggcacagctcacacccaggaggaatttctgccctcacctgatgccttctgcaaaactcacgtcctaatgcccagccagggctcagagttttcattaagcagtctgtatatttttctaagataacaaaataatttctccaaaggctttggtataattcaaagatagctagttagattcatttgcaaaatggcacacacctgaaatcccagcactcagaaggtagaggcacaaggatcaggagttcgaggccaacctagtccatatgtggagtttgaggtcagttggatctcattatctccaacccccaaaagaagccaaggaacggctcagtaggcaaagtgcttgctgtgcaaagatgaggacctgagcttggactccagcacccacataaagagacatcacagtaaggattgcaactccagcattctagttcctggggaaccactatactgctgaaggcagagctctatgccttgtaacagaataaacaaagatgctcaatggataaacatactgacacacatgtaggatggactcaacattctgtgttcagagtctttgaaggagtcattgtaagcaaaggcagaaacctcctcaatgacatcccaaagattcctgccagtgccccttctcctgtgtcatcatacagcccaaaagctggggtccacaccatgaagaaactccacatgacacccaaaggtttgtctctctgtccctggagcatagggtgagaatgagaagcctgctacttctgattctctggtttctgagcctcaagtagttcaggctggcctagaattcactgtgtagcccaggctagtcttgaactcttgatcctcctgcctccacccccaccaagtgttggggttataggagtgtgatgccactcctggtttattcagtattgggattgaaaccaggccagcactctacaactctacctcatcccagcccacttctggtccttcatacagccaactatcttcctgctacttataataaatgcttccagtcctttctgctgcccttctccaggctaagagaagaaaggatgaaggaagaggaatgacaatccatgctatgacaactaaatggtagctaaaaataaaacaaccctttgctttaattacagtgatacatacacttttgaaacttttccagaagcttttctgaatggcaaaggtagttcactgaaactactgacatagaataaaatccaccttagagaataaagcacatcttaatcctcaactcatcaagagtcataaaaacacagcacacaccaatgacatacttgtgaacttacattcctgttctaaaaatcaagggtgaatcacattgcaaccaggaaactgcccttgcctgggactcaggggcagctgccaaagcacagaactggtaagtttacgaggagactccaagttcccgatatcttcccccaagattggacctttcaactctttttctctttttattcttttaaattaaaagatgtgtgcgttgtgtgtgtgtgtgcgcacgcgcttgtgactgcaaatgctgccaagtgaacttggacaagcattactgcatct

SEQ ID NO: 160 - DNA, 180 bp, Homo sapiens
  ref <302>: TITLE: Human transforming growth factor
  ref <308>: DATABASE ACCESSION NUMBER: I01190.1
  ref <309>: DATABASE ENTRY DATE: 1993-05-21
  ref <310>: PATENT DOCUMENT NUMBER: US4742003
  ref <311>: PATENT FILING DATE: 1985-05-14
  ref <312>: PUBLICATION DATE: 1988-05-03
  ref <313>: RELEVANT RESIDUES IN SEQ ID NO: (1)..(180)
gatctgagccctgcatctttcctctccccagcagacccgcccgtggctgcagcagtggtgtcccattttaatgactgcccagattcccacactcagttctgcttccatggaacctgcaggtttttggtgcaggaggacaagccagcatgtgtgtaagtatcccctgttctcctggagatc

SEQ ID NO: 161 - DNA, 129 bp, Homo sapiens
  ref <302>: TITLE: Human-derived tumor cell growth inhibitors
  ref <308>: DATABASE ACCESSION NUMBER: AR019352
  ref <309>: DATABASE ENTRY DATE: 1998-12-05
  ref <310>: PATENT DOCUMENT NUMBER: US5783414
  ref <311>: PATENT FILING DATE: 1996-06-02
  ref <312>: PUBLICATION DATE: 1998-06-21
  ref <313>: RELEVANT RESIDUES IN SEQ ID NO: (1)..(129)
cagttcagacagaagacaatccacgtgtggctcaagtgtcaataacaaagtgtagctctgacatgaatggctattgtttgcatggacagtgcatctatctggtggacatgagtcaaaactactgcaggt

SEQ ID NO: 162 - DNA, 120 bp, Homo sapiens
  ref <302>: TITLE: Human-derived tumor cell growth inhibitors
  ref <308>: DATABASE ACCESSION NUMBER: AR019354
  ref <309>: DATABASE ENTRY DATE: 1998-12-05
  ref <310>: PATENT DOCUMENT NUMBER: US5783417
  ref <311>: PATENT FILING DATE: 1996-06-02
  ref <312>: PUBLICATION DATE: 1998-06-21
  ref <313>: RELEVANT RESIDUES IN SEQ ID NO: (1)..(120)
cagacagaagacaatccacgtgtggctcaagtgtcaataacaaagtgtagctctgacatgaatggctattgtttgcatggacagtgcatctatctggtggacatgagtcaaaactactgc

SEQ ID NO: 163 - DNA, 129 bp, Mus musculus
  ref <302>: TITLE: Human-derived tumor cell growth inhibitors
  ref <308>: DATABASE ACCESSION NUMBER: AR019353
  ref <309>: DATABASE ENTRY DATE: 1998-12-05
  ref <310>: PATENT DOCUMENT NUMBER: US5783417
  ref <311>: PATENT FILING DATE: 1996-06-02
  ref <312>: PUBLICATION DATE: 1998-06-21
  ref <313>: RELEVANT RESIDUES IN SEQ ID NO: (1)..(129)
tagttcagatggaagacgatccccgtgtggctcaagtgcagattacaaagtgtagttctgacatggacggctactgcttgcatggccagtgcatctacctggtggacatgagagagaaattctgcagat

SEQ ID NO: 164 - DNA, 1299 bp, Homo sapiens
  ref <308>: DATABASE ACCESSION NUMBER: BC035806
  ref <309>: DATABASE ENTRY DATE: 2003-03-04
  ref <313>: RELEVANT RESIDUES IN SEQ ID NO: (1)..(1299)
gacacagccaacgtggggtcccttctaggctgacagccgctctccagccactgccgcgagcccgtctgctcccgccctgcccgtgcactctccgcagccgccctccgccaagccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtaatatgtcagaaataaacaaacacagtttgtaaaattttgttttatagatttaggggtacaagtgcagatttgctagtggatatattcagtagtggtgaagtctgagcttttagagtacctacccctcaaatagtgtgcatggaacccattaggtaatttttcatcccttaacccccccaaaactcttctaccttttgaagtctccagagtctattactccactctctatgacaatgtgtacacattatttagctcccacttgtgagaacatgtgataaacaaatgcagttttactctttgtatttctatttttataatttgaaattaccctatatttccatgggctgttaaatgcagtatatatattattagaaacttttctgagtttttaaaaattaggtagtaaatagtagcttttaaattgcacacatatgtcagaggtgcagagcagggaggacttctgatgcttctcacacttgccaagatggtgtctctctgctttggatcttttccttcaatttctatatcaggtattgttttaagaattgattccaggccggacgcgttggctcatgcctgtaatcccagcactttgggaggccgaggcgggcggatcacggggtcaggagatcaagaccatcctggcgaacacggtgaaaccccgtctctactaaaaatacaaaaaaaaaaaaaattagccaggggtagtggcggacgcctgaagtcccagctactcgggaggctgaggcaggagaatggcatgaacccggggggtggagcttgcagtgagcggagatcatgccactgtactccagcctgggcaacacagcgagactccgtctcaaaaaaaaaaaaaaaaaaaaaaaaaaaaaaaaaaaaaaa

SEQ ID NO: 165 - DNA, 1215 bp, Homo sapiens
  misc_feature (554)..(839): n = undefined nucleitide
  ref <308>: DATABASE ACCESSION NUMBER: BM561909
  ref <309>: DATABASE ENTRY DATE: 2002-02-20
  ref <313>: RELEVANT RESIDUES IN SEQ ID NO: (1)..(1215)
taatacgaagacacagccaacgtggggtcctttctcggctgacagccgctctccagccactgccgcgagcccgtctgctcccgccctgcccgtgcactctccgcagccgccctccgccaagccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtaatatgtcagaaataaacaaacacagtttgtaaaattttgttttatagatttaggggtacaagtgcagatttgctagtggatatattcagtagtggtgaagactgctattactccatgtgcttcccgcnnnnnnnnnnnnnnnnnnnnnnnnnnnnnnnnnnnnnnnnnnnnnnnnnnnnnnnnnnnnnnggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccnnnnnnnnnnncnnngnnnnngnnnnnnngnnnnnnnnnnnnngttnttttngaaactttttttttgaggtttttaaaaaaattaggggtagtaaaaatagggaggttttttaaaatttgccccaccattatgtccaaaagtggccacaagtcaggaaaggaaccttttggagggcttttctcccccctttgcccccggaaggggggtcctcctccgggccttgggaatcttttttcccttacattttccaaaattccggggattttgttttttaaaaaaatggagatttccccgcgccccggacgccgtatggggcttcatggccctggaaaccccaccccactcttttgtggggggtcccgaggcaggggggggggaattccgcgggggccccggggaaattacaaacaccctccccctggggcga

SEQ ID NO: 166 - DNA, 549 bp, Homo sapiens
  misc_feature (355)..(355): n = undefined nucleitide
  ref <308>: DATABASE ACCESSION NUMBER: AA706226
  ref <309>: DATABASE ENTRY DATE: 1999-01-12
  ref <313>: RELEVANT RESIDUES IN SEQ ID NO: (1)..(549)
atcccggggagaaagccacccggcccaagttgaagaagatgaagagccagacgggacaggtgggtgagaagcaatcgctgaagtgtgaggcagcagcgggtaatccccagccttcctaccgttggttcaaggatggcaaggagctcaaccgcagccgagacattcgcatcaaatatggcaacggcagaaagaactcacgactacagttcaacaaggtgaaggtggaggacgctggggagtatgtctgcgaggccgagaacatcctggggaaggacaccgtcggaggccggctttacgtcaacagcgtgacgaccaccctgtcatcctggtcggggcacgcccggaagtgcaacgngacagccaagtcctattgcgtcaatggaggcgtctgctactacatcgagggcatcaaccagctctcctgcaaggcacctgggctgcactgcttagaacttggtacccagagccaccacttccccatctcagcctcccctggttccagccaaggttcctggaaccaacttccccaacaccctttgtcagccctcg

SEQ ID NO: 167 - DNA, 362 bp, Homo sapiens
  misc_feature (323)..(323): n = undefined nucleitide
  ref <308>: DATABASE ACCESSION NUMBER: BX089049
  ref <309>: DATABASE ENTRY DATE: 2003-01-23
  ref <313>: RELEVANT RESIDUES IN SEQ ID NO: (1)..(362)
agcacagctctgaggacctggtgttctgaccgcatctccaccagggctgccctctcccccgagggctgacaaagggtgttggggaagttggttccaggaaccttggctggaaccaggggaggctgagatggggaagtggtggctctgggtaccaagttctaagcagtgcagcccatgtgccttgcaggagagctggttgatgccctcgatgtagtagcagacgcctccattgacgcaataggacttggctgtctcgttgcacttccgggcgtgccccgaccaggatgacagggtggtgctcacgctgttgacgtaaagccggncccggacggtgtccttccccaggatgttctcggcctcgc

SEQ ID NO: 168 - DNA, 458 bp, Mus musculus
  ref <308>: DATABASE ACCESSION NUMBER: AI152190
  ref <309>: DATABASE ENTRY DATE: 1998-09-30
  ref <313>: RELEVANT RESIDUES IN SEQ ID NO: (1)..(458)
gtgtgaggcagcggcgggaaacccccagccctcctatcgctggttcaaggatggcaaggaactcaaccggagtcgtgatattcgcatcaagtatggcaatggcagtgagcaccactctgtcatcctggtcgggacatgcccggaagtgcaatgagaccgccaagtcctactgtgtgaatggaggcgtgtgctactacatcgagggcatcaaccagctctcctgcaaaggctgaggagctgtaccagaagagagtgctgacaattactggtatctgtgtggccctgctggtcgtgggcatcgtctgtgtggtcgcctactgcaagaccaaaaaacagaggaggcagatgcatcatcatctccggcagaacatgtgcccagcccaccagaaccgaagcctggccaacgggccagccaccctcggctggaccatgaggagaccagatggcagattaatctc

SEQ ID NO: 169 - DNA, 539 bp, Danio rerio
  ref <308>: DATABASE ACCESSION NUMBER: AL918370
  ref <309>: DATABASE ENTRY DATE: 2004-07-06
  ref <313>: RELEVANT RESIDUES IN SEQ ID NO: (1)..(539)
ccaccagcagagccacgcagatgccagttatcgtcagcactcgttttggtacagctcctcagccttgtagaaaccggccataacggaggtttgacagcgttcgccggtatagtcatttggacacttgcaggacagctgatttataccatgtatgaaataacagtctccaccgttgatgcagtatgtcttctcagtttcattgcacttcctggcatgacttgagcccggagacaatgtggtggttatgctttggacgctgacgaagctggtggcgttttctctgcccagcgagttctccaccacacaggtgtagttcccagaatcctccagtctgactttgctaatgtgaagctttgagtttttcttgttggttttgattttgacggttttcttttggcgaagctggctgccatctttgtaccagttgaaggaggggctcgggttgcccacagcttcacacttcagtgtcaactttttaccttcctggagccactgagaatccatgggcttcacctttggagctgatgcgcagtctttac

SEQ ID NO: 170 - DNA, 654 bp, Gallus gallus
  ref <308>: DATABASE ACCESSION NUMBER: BU465274
  ref <309>: DATABASE ENTRY DATE: 2002-11-29
  ref <313>: RELEVANT RESIDUES IN SEQ ID NO: (1)..(654)
cacgctgggagatgagtgctgtggtgcccagctgtgaggtgcctgggctggcagtgcttctccctctctccctctgcaggggaaagaaagaagggactttttctttctctgaagtagaagttcagattttgatggtaagggagctgatgtggaggcctggccttaaggaaggctttcagtaggcagtacagtctttggagctgctgcagcagacctggcggttgtctaccttgcaatttgagtatgacagaagagtagcctgtggattccactatactacaacgtattccactgagcgatctgagcactttaagccatgcaaagacaaggatcttgcatactgtctcaacgagggggaatgctttgtgattgaaaccttaacaggatcacataaacactgccgcagcaattgcccttctggtgttttctgctggtgacctgtctgaatagatgttcttccagaggtggttgtggtttggggcattgatgctgggaagaggattaccaggaagagctcagctgttccttcattgctcagtccacgtttataaagaaggatggacagtgacctgtgagcaagcttgtttgcaaaagaaagcattatctgttggtaacttttgcaataaaaaatatttcttgtattactctaaaaaa

SEQ ID NO: 171 - DNA, 758 bp, Gallus gallus
  misc_feature (4)..(4): n = undefined nucleitide
  ref <308>: DATABASE ACCESSION NUMBER: BU372401
  ref <309>: DATABASE ENTRY DATE: 2002-11-28
  ref <313>: RELEVANT RESIDUES IN SEQ ID NO: (1)..(758)
gcanggcgggaggcgccgcgcggtcgctgtccgcgggcagacagcggcattacataaccgcgtacagagagcagctgcgggattacacgatgcagattagcggcggcgttgattcagcagatgccctgtgcgtgtgtgagggggattacggcggcgcggggcagaaccgccgtgcgggtgccgttttagaagaatagcttctgaccaagaattagaattgttggaataatatgcgaacagatcatgaagaactctgtggcaccagttatggatctttttgtctaaatggaggcatttgctatatgattcctactgtacccagtccattctgcagacatcttccgaaagcagcaaaccaagcttcagccttacataagtcagtcttctctatcttcgttttacatacagacaccactgcactcccaagctgccatttaatgcctgctcatttctatacgcaatgaaagataactagaaaatccgtatttcaaggctatcctccatttctacatccctgcaaactacctaagaacaattagatggaacaggattgtctacaacattgttatcacaaaggaggctatcttatggatggaatttcttttttctcagatgtattacttaccagcaaggaaggtagttctgtttgaatcttctcaataaacaccacatttcctgtttcaggttgggtgggaactattcttcaaacggaggaggtttatgtgttcctttcgttcctataatgtctcaataatgag

SEQ ID NO: 172 - DNA, 547 bp, Mus musculus
  ref <308>: DATABASE ACCESSION NUMBER: BE624667
  ref <309>: DATABASE ENTRY DATE: 2000-08-24
  ref <313>: RELEVANT RESIDUES IN SEQ ID NO: (1)..(547)
gttgctgaagtcctcagtgttcaaacacttgtgaaacgctgcatgtctagcaaaattttctttttttatgggaatataaatttctgttgaggtgctgattttcaaccttaattcttccatcaagaatgaaactatttaaaaattaagatgccaacagatcacgagcagccctgtggtcccaggcacaggtcattttgcctcaatggggggatttgtattgatccctactatccccacccattctgtaggttttatcatttgtttctaagacattgcctacttaaaccattcgtgcaattgggcaccttggtgtacccagtgtttctgaaggagttattccattgacgcgccccaagttcttcatgcagtggtgttcctgaatgcttgaaatctgttttctgcgaatccttggtgggatggctagaaacctgtgaaaaatcatgaaatcaccaaataccatgtgatgtgtatagtctcttctcctctccactgacagcttaatcaggggaaagggactgttgctgcttctctttgtcttattcccagt

SEQ ID NO: 173 - DNA, 233 bp, Homo sapiens
  ref <308>: DATABASE ACCESSION NUMBER: BE064716
  ref <309>: DATABASE ENTRY DATE: 2000-06-09
  ref <313>: RELEVANT RESIDUES IN SEQ ID NO: (1)..(233)
cggatgtatcccaacaccgtcacggaaatattctgctgacattgcatgttactgcttccaggtgctctatatatttgcattctccgtgaatgcagaaattttgaaattctgcatcacatggatttttcttctttctgtttcttctattttttccatttttgcctccctttttctttcttttgggtttatctgaagtattttcactttccggcttgtgttgggcgataacatca

SEQ ID NO: 174 - DNA, 533 bp, Homo sapiens
  misc_feature (7)..(7): n = undefined nucleitide
  ref <308>: DATABASE ACCESSION NUMBER: BG194271
  ref <309>: DATABASE ENTRY DATE: 2001-04-21
  ref <313>: RELEVANT RESIDUES IN SEQ ID NO: (1)..(533)
ccctagntgccaccacacaatcaaagtggaaaggccactcctctaggtgccccaagcaatacaagcattactgcatcaaagggagatgccgcttcgtggtggccgagcagacgccctcctgtgtccctctccggaaacgtcgtaaaagaaagaagaaagaagaagaaatggaaactctgggtaaagatatgactcctatcaatgaagatattgaagagacaaatattgcttataaggctatgaagttacctccaggttggtggcaagctgcaaagtgccttgctcatttgaaaatggacagaatgcgtctcaggaaaacagctagtagacatgaattttaaataatgtatttactttttatttgcaacttcagtttgtgttattattttttaataagaacattaattatatgtatattgtctagtaattgggaaaaaagcaactggttaggtagcaacaacagaagggaaatttcaataacctttcacttaagtattgtcaccaggattactagtcaaacaaaaaaaaaaaaa

SEQ ID NO: 175 - DNA, 689 bp, Mus musculus
  misc_feature (671)..(671): n = any nucleotide
  ref <308>: DATABASE ACCESSION NUMBER: BY735030
  ref <309>: DATABASE ENTRY DATE: 2002-12-17
  ref <313>: RELEVANT RESIDUES IN SEQ ID NO: (1)..(689)
gcagattatttgtttaccacttagaacacaggatgtcagcgccatcttgtaacgacgaatgtgggggcggctcccaacacttcaccatggttttgaccttgtcatgaccagttattttctggcttatctccactaatcttgggagcctcagcaccagccctgagttcatatcacaccaccaaagtctttgacctggaagagctttaacttcctaagcctcctgcttccactgggcagcactggtacccggagaatcctgtgtcccttgtctactccatcctgttctgcaggtcttgcaattctccactgtgtggtagcagatgggaacacaaccagaacaccagaaaccaatggctctctttgtggagctcctggggaaaactgcacaggtaccacccctagacagaaagtgaaaacccacttctctcggtgccccaagcagtacaagcattactgcatccatgggagatgccgcttcgtggtggacgagcaaactccctcctgcatggcccggctcagcatctacttgtggagaaactgacgcagactttcctcctgaaatctgaatatgagaaaccaggtccagttctgccctgctggtgtcccaactcccttgtgcaagaaaaggcgattctaatcgtgttaggatgctcgatagttccaatcatctnctgggtgtttcaatgaaa

SEQ ID NO: 176 - DNA, 1196 bp, Cercopithecus aethiops (African green monkey)
  ref <308>: DATABASE ACCESSION NUMBER: X89728
  ref <309>: DATABASE ENTRY DATE: 2005-04-18
  ref <313>: RELEVANT RESIDUES IN SEQ ID NO: (1)..(1196)
gcccagcggaatctcttgagtcccaccgcccagctccggtgccagcgcccagtggccgccgcttcgaaagtgactggtgcctcgccgcctcctctcggtgcgggaccatgaagctgctgccgtcggtggtgctgaagctccttctggctgcagttctttcggcactggtgactggcgagagcctggagcagcttcggagagggccagctgctggaaccagcaacccggacccttccactggatctacggaccagctgctacgcctaggaggcggccgggaccggaaagtccgtgacttgcaagaggcagatctggaccttttgagagtcactttatcctccaagccacaagcactggccacaccaagcaaggaggagcacgggaaaagaaagaagaaaggcaagggactagggaagaagagggacccatgtcttcggaaatacaaggacttctgcatccacggagaatgcaaatatgtgaaggagctccgggctccctcctgcatggcagctgggcagaaagatgttacttgatttgtttggtttgtcctgtgatgaaagaggcctggtagctcagcgttcagaggccaaaggccagagctgccacccaggttaccatggagagaggtgtcatgggctgagcctcccagtggaaaatcgcttatatacctatgaccatacaactatcctggctgtggtggccgtggtgctgtcctctgtctgtctgctggtcatcgtggggcttctcatgtttaggtaccataggagaggtggttatgatgtggaaaacgaagagaaagtgaagttgggcatgactaattcccactgagagacttgtgctcaaggaatcagctggtgactgctacctctgagaagacacaaggtgatttcagattgcagaggggaaagacgtcacatctagccacaaagactccttcatccccagtcgccatctaggattgggcctcccataattgctttgccaaaataccagagccttcaagtgccaaaccgagtatgtctgatagtatctgggtgagaagaaagcaaaagcaagggaccttcatgcccttctgattcccctccaccaagccccacttccccttataagtttgtttaagcactcacttctggattagaatgccggttaaattccatatgctccaggatctttgactgaaaaaaaaaaaaaaaaaa

SEQ ID NO: 177 - DNA, 564 bp, Homo sapiens
  ref <302>: TITLE: EGF-like nucleic acids and polypeptides and uses thereof
  ref <308>: DATABASE ACCESSION NUMBER: BD274363
  ref <309>: DATABASE ENTRY DATE: 2003-07-17
  ref <310>: PATENT DOCUMENT NUMBER: JP2002530064
  ref <311>: PATENT FILING DATE: 1999-11-19
  ref <312>: PUBLICATION DATE: 2002-09-17
  ref <313>: RELEVANT RESIDUES IN SEQ ID NO: (1)..(564)
acggggtccgagaaagttaagcaactacaggaaatggctttgggagttccaatatcagtctatcttttattcaacgcaatgacagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactgtgaggcctttgtgaagaattttcatcaaggcatctgtagagatcaagtgagcccaaaattaaagttttcagatgaaacaacaaaacttgtcaagctgactagactcgaaaatatggaaagttggggatcacaatgaaatgagaagataaaatcagcggtggcccttagactttgccatccttaaggagtgatggaagccaagtgaacaagcctcagtgacacaagtcaaattcataggttcactctggg

SEQ ID NO: 178 - DNA, 387 bp, Homo sapiens
  ref <302>: TITLE: HUMAN GENES AND GENE EXPRESSION PRODUCTS XVI
  ref <308>: DATABASE ACCESSION NUMBER: AX261946
  ref <309>: DATABASE ENTRY DATE: 2001-10-26
  ref <310>: PATENT DOCUMENT NUMBER: WO0172781
  ref <311>: PATENT FILING DATE: 2001-03-27
  ref <312>: PUBLICATION DATE: 2001-10-04
  ref <313>: RELEVANT RESIDUES IN SEQ ID NO: (1)..(387)
ggcacgagggaggctctttgttatagatgcttttgcccccttaatacagcaatgagagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactgtgaggcctttgtgaagaattttcatcaaggcatctgtagagatcagtgagcccaaaattaaagttttcagatgaaacaacaaaacttgtcaagctgact

SEQ ID NO: 179 - DNA, 389 bp, Homo sapiens
  ref <302>: TITLE: HUMAN GENES AND GENE EXPRESSION PRODUCTS XVI
  ref <308>: DATABASE ACCESSION NUMBER: AX261991
  ref <309>: DATABASE ENTRY DATE: 2001-10-26
  ref <310>: PATENT DOCUMENT NUMBER: WO0172781
  ref <311>: PATENT FILING DATE: 2001-03-27
  ref <312>: PUBLICATION DATE: 2001-10-04
  ref <313>: RELEVANT RESIDUES IN SEQ ID NO: (1)..(389)
ggcacgaggaaagttaagcatctacaggttatggctttgggagttccaatatcagtctatcttttattcaacgcaatgacagcactgaccgaagaggcagccgtgactgtaacacctccaatcacagcccagcaaggtaactggacagttaacaaaacagaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactgtgaagcctttgtgaagaattttcatcaaggcatctgtagagatcagtgag

SEQ ID NO: 180 - DNA, 409 bp, Homo sapiens
  ref <302>: TITLE: EGF-like nucleic acids and polypeptides and uses thereof
  ref <308>: DATABASE ACCESSION NUMBER: BD274361
  ref <309>: DATABASE ENTRY DATE: 2003-07-17
  ref <310>: PATENT DOCUMENT NUMBER: JP2002530064
  ref <311>: PATENT FILING DATE: 1999-11-19
  ref <312>: PUBLICATION DATE: 2002-09-17
  ref <313>: RELEVANT RESIDUES IN SEQ ID NO: (1)..(409)
aactacaggaaatggctttgggagttccaatatcagtctatcttttattcaacgcaatgacagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactgtgaggcctttgtgaagaattttcatcaaggcatcttgtagagatcaagtgagcccaaaattaaagttttcagatgaaacaacaaaacttgtcaagctgactagactcgaaaatatg

SEQ ID NO: 181 - DNA, 568 bp, Homo sapiens
  ref <302>: TITLE: Compositions isolated from skin cells and methods for their use
  ref <308>: DATABASE ACCESSION NUMBER: BD209747
  ref <309>: DATABASE ENTRY DATE: 2003-07-17
  ref <310>: PATENT DOCUMENT NUMBER: JP2002512798
  ref <311>: PATENT FILING DATE: 1999-04-29
  ref <312>: PUBLICATION DATE: 2002-05-08
  ref <313>: RELEVANT RESIDUES IN SEQ ID NO: (1)..(568)
ccgtcagtctagaaggataagagaaagaaagttaagcaactacaggaaatggctttgggagttccaatatcagtctatcttttattcaacgcaatgacagcactgaccgaagaggcagccgtgactgtaacacctccaatcacagcccagcaaggtaactggacagttaacaaaacagaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactgtgaggcctttgtgaagaattttcatcaaggcatctgtagagatcagtgagcccaaaattaaagttttcagatgaaacaacaaaacttgtcaagctgactagactcgaaaataatgaaagttgggatcacaatgaaatgagaagataaaattcagcgttggcctttagactttgccatccttaaggagtgatggaagccaagtgaacaagcc

SEQ ID NO: 182 - DNA, 282 bp, Homo sapiens
  ref <302>: TITLE: EGF-like nucleic acids and polypeptides and uses thereof
  ref <308>: DATABASE ACCESSION NUMBER: bd274362
  ref <309>: DATABASE ENTRY DATE: 2003-07-17
  ref <310>: PATENT DOCUMENT NUMBER: jp2002530064
  ref <311>: PATENT FILING DATE: 1999-11-19
  ref <312>: PUBLICATION DATE: 2002-09-17
  ref <313>: RELEVANT RESIDUES IN SEQ ID NO: (1)..(282)
atggctttgggagttccaatatcagtctatcttttattcaacgcaatgacagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactg

SEQ ID NO: 183 - protein, 32 aa, Homo sapiens
NSDSECPLSHDGYCLHDGVCMYIEALDKYACK

SEQ ID NO: 184 - protein, 32 aa, Homo sapiens
GHARKCNETAKSYCVNGGVCYYIEGINQLSCK

SEQ ID NO: 185 - protein, 32 aa, Homo sapiens
NSYPGCPSSYDGYCLNGGVCMHIESLDSYTCK

(19) United States
(12) Patent Application Publication
(10) Pub. No.: US 2009/0131308 A1
(43) Pub. Date: May 21, 2009

(54) Splice Variants of ErbB Ligands, Compositions and Uses Thereof

(75) Inventor: Daniel Harari, Rehovot (IL)

Correspondence Address: BROWDY AND NEIMARK, P.L.L.C.;624 NINTH STREET, NW, SUITE 300, WASHINGTON, DC, 20001-5303, US

(73) Assignee: AGOS BIOTECH LTD., Rehovot (IL)

(21) Appl. No.: 10/568,806

(22) PCT Filed: Aug. 19, 2004
(86) PCT No.: PCT/IL04/00759

§ 371 Date: Oct. 3, 2008

Related U.S. Application Data
(60) Provisional application No. 60/495,898, filed on Aug. 19, 2003.

(51) Int. Cl. A61K 38/16; C07K 14/00; C12N 15/11; A61K 31/7088; C12N 15/00; C12N 5/06

(52) U.S. Cl. 514/12; 530/324; 530/350; 536/23.5; 435/320.1; 435/325; 514/44; 435/377

(57) ABSTRACT

The present invention relates to nucleic acid and amino acid sequences of previously unknown ErbB ligands that are splice variants of previously known ErbB ligands, to compositions comprising these sequences and uses thereof in the diagnosis, treatment, and prevention of diseases and disorders mediated by ErbB receptors. Specifically, the present invention relates to splice variants lacking the C-loop of an intact EGF domain.

### FIELD OF THE INVENTION

The present invention relates to nucleic acid and amino acid sequences of ErbB ligands that are splice variants of previously known ErbB ligands and to compositions comprising these sequences, and uses thereof in the diagnosis, treatment, and prevention of diseases and disorders mediated by ErbB receptors.

### BACKGROUND OF THE INVENTION

Receptor tyrosine kinases play a key role in the dissemination of cell to cell signaling in organisms typically upon activation via specific activating ligands. Type-1 tyrosine kinase receptors, also known as ErbB/HER proteins, comprise one such receptor tyrosine kinase family, of which the epidermal growth factor receptor (EGFR; ErbB-1) is the prototype. The mammalian/human ErbB family to date consists of four known receptors (ErbB-1 to ErbB-4). Upon ligand binding the receptors dimerize, transducing their signals by subsequent autophosphorylation catalyzed by an intrinsic cytoplasmic tyrosine kinase, and recruiting downstream signaling cascades (reviewed by Yarden and Sliwkowski 2001).

### The ErbB Ligands

The ErbB receptors are activated by a large number of ligands. This ligand family is encoded in humans by at least eleven independent genes and their splice variants and include the Neuregulins (NRG-1, NRG-2, NRG-3 & NRG-4), the Epidermal Growth Factor (EGF), TGF alpha, Betacellulin, Amphiregulin, Heparin-Binding EGF (HB-EGF), Epiregulin and Epigen (reviewed in Harari et. al. 1999; Harris et. al 2003). These ligands each have a selective repertoire of receptors to which they bind preferentially, each with its own array of differential binding affinities. Typically but not exclusively, the Neuregulins preferably bind to ErbB3 and/or ErbB4, whereas the remaining ligands bind ErbB1. Upon ligand binding, receptor homodimers and heterodimers are typically recruited. ErbB2, which is bound by no known ligand, nevertheless can be actively recruited in a ligand-dependent manner, as a heterodimer. Depending upon the activating ligand, most homodimeric and heterodimeric ErbB combinations can be stabilized upon ligand binding, thus allowing a complex, diverse downstream signaling network to arise from these four receptors. The choice of dimerization partners for the different ErbB receptors, however, is not arbitrary. Spatial and temporal expression of the different ErbB receptors do not always overlap in vivo, thus narrowing the spectrum of possible receptor combinations that an expressed ligand can activate for a given cell type (reviewed in Harari et al. 1999; Harari and Yarden 2000).

A hierarchical preference for signaling through different ErbB receptor complexes takes place in a ligand-dependent manner. Of these, ErbB-2-containing combinations are often the most potent, exerting prolonged signaling through a number of ligands, likely due to an ErbB-2-mediated deceleration of ligand dissociation. In contrast to possible homodimer formation of ErbB-1 and ErbB4, for ErbB-2, which has no known direct ligand, and for ErbB-3, which lacks an intrinsic tyrosine kinase activity, homodimers either do not form or are inactive. Heterodimeric ErbB complexes are arguably of importance in vivo. For example, mice defective in genes encoding either NRG-1, or the receptors ErbB-2 or ErbB4, all result in identical failure of trabeculae formation in the embryonic heart, consistent with the notion that trabeculation requires activation of ErbB-2/ErbB-4 heterodimers by NRG-1 (reviewed in Harari et al. 1999).

The repertoire of ErbB ligands and receptors differs between simpler and more complex organisms. In the worm C. elegans, a single ErbB ligand and receptor are encoded (Moghal and Sternberg 2003). Drosophila melanogaster likewise encodes a single ErbB receptor gene but has an expanded ligand family of four agonists (Vein, Gurken, Spitz and Keren) and a single antagonist, named Argos (Shilo 2003; Table 1). In mammals this has further expanded to genes encoding at least eleven ligands and four receptors. However, no mammalian inhibitory Argos-like ErbB ligand has been described to date. These known ErbB ligands are listed in Table 1.

TABLE 1
Agonist and Antagonist Ligands of the
ErbB Receptor Tyrosine Kinase Family

| | Agonist | Antagonist |
|---|---|---|
| C. elegans | Lin-3 | |
| Drosophila | Vein | Argos |
| | Gurken | |
| | Spitz | |
| | Keren | |
| Mammals | NRG-1 (alpha and beta isoforms) | |
| | NRG-2 (alpha and beta isoforms) | |
| | NRG-3 | |
| | NRG-4 | |
| | EGF | |
| | TGF-alpha | |
| | Betacellulin | |
| | Amphiregulin | |
| | Heparin-Binding EGF (HB-EGF) | |
| | Epiregulin | |
| | Epigen | |

### A Receptor Modulating EGF Domain Motif of the ErbB Ligand Family

Across an evolutionarily diverse selection of organisms, ErbB ligands each harbor a conserved motif, namely the EGF domain. The EGF domains (including the antagonist ligand Argos derived from an invertebrate) are critical for receptor binding and modulation. Most ligands share the common feature of harboring a single EGF domain and a single transmembrane domain. The EGF domain is found adjacent to the transmembrane domain and on its amino terminal side, thus constituting a component of the ligand ectodomain. For numerous ligands the EGF domain has been demonstrated to be both necessary and sufficient to confer receptor binding and activation.

Exceptionally, the Epidermal Growth Factor includes nine extracellular EGF domains of which only the ninth EGF domain, i.e., that in closest proximity to the transmembrane domain has been shown to confer receptor binding (Carpenter and Cohen 1990). The transmembrane domain tethers the ligand to the cell surface. A complex process of post-translational proteolytic cleavage of the extracellular domain is required to release the tethered EGF domain which in many instances is critical for ligand activation (Harris et al. 2003). However, there do exist in nature ligands devoid of a transmembrane domain, as is the case for some splice variants of NRG-1 for example. Additionally, a variant of NRG-1 (Heregulin gamma; NRG1 gamma) with a truncated EGF domain has been described, albeit reportedly unlikely to be bioactive (Falls 2003).

The ErbB-receptor-binding EGF domains harbor six invariant cysteine residues which are responsible for the formation of three disulfide bridges (considered to form the bridges Cys1-Cys3, Cys2-Cys4 and Cys5-Cys6) denoted as loops A, B and C (FIG. 1 from Harari and Yarden 2000). Besides the conserved cysteines, the receptor-binding EGF domain of these ligands encode numerous conserved and semi-conserved residues, including a Glycine and Arginine residue proximal to Cys-6 (boxed residues in FIG. 1 corresponding to Gly-40 & Arg-42 or Gly-39 & Arg41 for synthetic peptides encoding the ligand-binding EGF domain of TGF-alpha and epidermal growth factor respectively as defined by others (Jorissen et al. 2003)). The conservation of these Glycine and Arginine residues are not coincidental. Substitutional mutagenesis of these residues severely compromises ligand binding or function (Campion and Niyogi 1994; Groenen et al. 1994; Summerfield et al. 1996).

### Insect Argos

Genetic evidence from flies, demonstrates that Argos acts as a negative regulator in EGFR signaling (Howes et. al, 1998). The Drosophila melanogaster ligand Argos contains an EGF domain which harbors a B-loop which is larger than that for the activatory ligands (FIG. 1). Despite this divergence from the remainder of the ErbB ligand family, it has been suggested that the Argos EGF domain binds directly to the Drosophila EGF Receptor (Jin et al. 2000; Vinos and Freeman 2000). The Argos EGF domain reportedly plays an essential role not just in receptor binding, but also in the ligand's antagonist function. A domain swap of the Argos EGF domain into the agonist ligand Vein, converted this activatory ligand into an inhibitor (Schnepp et al. 1998). Furthermore, Argos blocks the binding of secreted Spitz to the Drosophila EGF receptor, suggestive that the inhibitory ligand competitively displaces agonist ligand binding (Jin et al. 2000). The assumption that Argos binds to the Drosophila EGFR and inhibits receptor function has been disputed recently when Argos was not found to bind directly to the EGF Receptor, but rather, conferred high affinity binding to the activatory ligand Spitz. This suggests an entirely different mechanism in which Argos inhibits EGF Receptor signaling by ligand sequestration and not by direct Argos—EGF Receptor binding (Mark Lemmon; The Fourth Dubrovnik Signaling Conference, FEBS, May 2004). In summary, although genetic evidence demonstrates that Argos acts as an inhibitory ligand of the EGF Receptor pathway, the general mechanism by which it exerts its inhibitory function still remains in dispute.

In the C-loop, Drosophila melanogaster Argos contains the canonical Glycine and Arginine residues typical for this ligand family (Boxed region; FIG. 1; equivalent to Gly39 and Arg41 of EGF (Groenen et al. 1994)). However, this otherwise invariant Arginine residue has been substituted to a Histidine, in Argos sequenced from Musca domestica, another insect species, demonstrating that absolute conservation at this residue is not required for Argos function (Howes et al. 1998). This finding has been re-represented in FIG. 2, as a multiple alignment for three insect species. The significance of the Arg to H is substitution in Musca domestica Argos should not be underestimated, especially if considering a model where Argos binds the EGF Receptor. A panel of substitution mutations of EGF Arg41 (or the corresponding Arg42 of TGF alpha) were shown to decrease ligand-receptor binding affinity by more than 100-fold (Campion and Niyogi 1994; Defeo-Jones et al. 1989; Engler et al. 1992). Replacement of the Argos C-loop with that from the stimulatory Drosophila ligand Spitz, results in the formation of a chimeric protein that retains moderate inhibitory activity (Howes et al. 1998). These findings do not provide a mechanistic understanding as to the action of Argos. However, they provide evidence to support the hypothesis that the C-loop of Argos cannot be considered responsible (or at least entirely responsible) for the inhibitory function of Argos.

ErbB ligands have been shown to be essential in induction and propagation of cell proliferation and are also involved in many other cell-signaling pathways in a wide variety of normal and malignant physiological events. Therefore, both agonists and antagonists of the ErbB signaling pathways have enormous therapeutic potential (reviewed by Mendelsohn and Baselga, 2003).

The above described ErbB ligands and methods of using same emphasize the phenomenon that different ErbB ligands may have different structure and function. Novel splice variants of ErbB ligands are likely to have a physiological role, whether systemic or tissue specific.

Therefore, there is a recognized need for, and it would be highly advantageous to isolate and characterize ErbB ligand splice variants, that include truncations, deletions, alternative exon splicing or translatable intronic sequences, which alter the composition, length or function of the receptor modulating EGF domain.

### SUMMARY OF THE INVENTION

The present invention provides novel ErbB ligand splice variants, including truncation variants, deletion variants, alternative exon usage, and intronic sequences, that each comprise at least one altered component of the EGF domain that affects ligand-mediated ErbB receptor activation. Without wishing to be bound by any particular mechanism or theory of action, the variant EGF domain may affect receptor activation directly through receptor binding, or indirectly by means of ligand sequestration, or by any other mechanism that alters ErbB receptor activation. The invention relates to isolated polynucleotides encoding these novel variants of ErbB ligands, including recombinant DNA constructs comprising these polynucleotides, vectors comprising the constructs, host cells transformed therewith, and antibodies that specifically recognize one or more epitope present on such splice variants.

It is an object of the present invention to provide vectors, including expression vectors containing the polynucleotides of the invention, cells engineered to contain the polynucleotides of the present invention, cells genetically engineered to express the polynucleotides of the present invention, and methods of using same for producing recombinant ErbB ligand splice variants according to the present invention.

It is a further object of the present invention to provide synthetic peptides comprising the novel amino acid sequences disclosed herein. It is explicitly to be understood that the novel splice variants disclosed herein as ErbB ligands, whether deduced from conserved genomic DNA sequences, deduced from cDNA sequences, or derived from other sources, may be produced by any suitable method involving recombinant technologies, synthetic peptide chemistry or any combination thereof.

It is a yet another object of the present invention to provide pharmaceutical compositions comprising the novel ErbB ligand splice variant or polynucleotide encoding same. It is yet further object of the present invention to provide methods for the diagnosis and treatment of ErbB receptor related diseases comprising administering to a subject in need thereof a pharmaceutical composition comprising as an active ingredient a novel ErbB ligand or a polynucleotide encoding same.

According to one aspect, the present invention provides ErbB ligand splice variant polypeptides and polynucleotides encoding same. Novel isoforms and putative isoforms of known ErbB ligands are disclosed, that are characterized in that they do not comprise the C-loop of the EGF domain. In other words, the unifying feature of the splice variants of the present invention is that they lack cysteines 5 and 6 of the invariant six cysteines of hitherto known ErbB ligand receptor-binding or receptor modulating EGF domains.

According to one embodiment, the present invention provides novel mature polypeptides having ErbB receptor agonist or antagonist activity, as well as fragments, analogs and derivatives thereof. According to some embodiments, the polypeptides of the present invention are of non-mammalian vertebrate origin. According to other embodiments, the polypeptides of the present invention are of mammalian origin.

According to other embodiments the polypeptides are of human origin.

According to a one embodiment the present invention provides a polypeptide comprising a splice variant of an ErbB ligand encoded by differential exon usage comprising a truncated EGF domain devoid of the C-loop of the EGF domain.

According to certain preferred embodiments the present invention provides ErbB ligand splice variants, comprising the sequence set forth in any one of SEQ ID NOs:73 to 84.

It is understood that the present invention includes active fragments, deletions, insertions, and extensions of these sequences with the proviso that any such extensions are absent the C-loop of the corresponding known EGF domain. According to certain specific embodiments, novel splice variants according to the present invention that comprise the truncated EGF domain are those having a sequence as set forth in any one of SEQ ID NOS: 93, 95-104, 109-121.

According to another embodiment the present invention provides polynucleotides encoding for the ErbB ligand splice variants, including an isolated polynucleotide comprising the sequence set forth in any one of SEQ ID NOS: 128-139 and SEQ. ID NOS:148, 150-159, 164-176.

It is to be understood that the present invention encompasses all active fragments, variants and analogs of the sequences disclosed herein that retain the biological activity of the sequence from which they are derived, with the proviso that said variants and analogs are devoid of the C-loop of the EGF domain.

The invention also provides a polynucleotide sequence which hybridizes under stringent conditions to the polynucleotide encoding the amino acid sequence set forth in any one of SEQ ID NOS:73 to 84 and SEQ ID NOS: 93, 95-104, 109-121, or fragments of said polynucleotide sequences. The invention further provides a polynucleotide sequence comprising the complement of the polynucleotide sequence encoding the amino acid sequence set forth in any one of SEQ ID NOS:73 to 84 and SEQ ID NOS: 93, 95-104, 109-121, or fragments or variants of said polynucleotide sequence.

According to some embodiments, the isolated polynucleotides of the present invention include a polynucleotide comprising the nucleotide sequence set forth in any one of SEQ ID NOS:128 to 139 and SEQ ID NOS:148, 150-159, 164-176, or fragments, variants and analogs thereof. The present invention further provides the complementary sequence for a polynucleotide having set forth in any one of SEQ ID NO:128 to 139 and SEQ ID NOS:148, 150-159, 164-176 or fragments, variants and analogs thereof. The polynucleotide of the present invention also includes a polynucleotide that hybridizes to the complement of the nucleotide sequence set forth in any one of SEQ ID NOS:128 to 139 and SEQ ID NOS:148, 150-159, 164-176 under stringent hybridization conditions.

According to yet another embodiment, the present invention provides an expression vector containing at least a fragment of any of the polynucleotide sequences disclosed. In yet another embodiment, the expression vector containing the polynucleotide sequence is contained within a host cell. The present invention further provides a method for producing the polypeptides according to the present invention comprising; a) culturing the host cell containing an expression vector containing at least a fragment of the polynucleotide sequence encoding an ErbB ligand splice variant including sequences encoding the variant EGF domain, under conditions suitable for the expression of the polypeptide; and b) recovering the polypeptide from the host cell culture.

According to another aspect the present invention also provides a method for detecting a polynucleotide which encodes an ErbB variant ligand in a biological sample comprising the steps of: a) hybridizing the complement of the polynucleotide sequence which encodes a polypeptide having the sequence set forth in any one of SEQ ID NOS:73 to 84 and SEQ ID NOS:93, 95-104, 109-121 to nucleic acid material of a biological sample, thereby forming a hybridization complex; and b) detecting the hybridization complex, wherein the presence of the complex correlates with the presence of a polynucleotide encoding an ErbB variant ligand in the biological sample. According to one embodiment the nucleic acid material of the biological sample is amplified by the polymerase chain reaction prior to hybridization.

According to yet another aspect the present invention provides a pharmaceutical composition comprising a polypeptide having the amino acid sequence set forth in any one of SEQ ID NOS:73 to 84 and SEQ ID NOS:93, 95-104, 109-121 or a polynucleotide encoding same, further comprising a pharmaceutically acceptable diluent or carrier.

According to further aspects the present invention provides a purified molecule or compound to prevent or inhibit the function of the ErbB ligand splice variant of the present invention. The inhibitor may be selected from the group consisting of antibodies, peptides, peptidomimetics and small organic molecules. The inhibitor, preferably a specific antibody, has a number of applications, including identification, purification and detection of variant ErbB ligand, specifically any antibody capable of recognizing an epitope present on the ErbB ligand splice variant devoid of the C-loop of the EGF domain, that is absent form the known counterparts that include the C-loop of the EGF domain.

According to one embodiment, the present invention provides a purified antibody which binds to at least one epitope of a polypeptide comprising the amino acid sequence set forth in any one of SEQ ID NOS:73 to 84 and 93, 95-104, 109-121, or specific fragments, analogs and variants thereof, with the proviso that the epitope is absent on the known counterpart ErbB ligands.

Further aspects of the present invention provide methods for preventing, treating or ameliorating an ErbB receptor related disease or disorder, comprising administering to a subject in need thereof a pharmaceutical composition comprising as an active ingredient an ErbB ligand splice variant, as disclosed hereinabove.

According to one embodiment, the present invention provides a method for preventing, treating or ameliorating an ErbB receptor related disease or disorder, comprising administering to a subject in need thereof a pharmaceutical composition comprising as an active ingredient a polypeptide comprising the sequence set forth in any one of SEQ ID NOS:73 to 84 and SEQ ID NOS:93, 95-104, 109-121.

According to another embodiment, the present invention provides a method for preventing, treating or ameliorating an ErbB receptor related disease or disorder, comprising administering to a subject in need thereof a pharmaceutical composition comprising as an active ingredient a polynucleotide encoding a polypeptide comprising any one of sequence set forth in SEQ ID NOS:73 to 84 and SEQ ID NOS: 93, 95-104, 109-121.

According to another embodiment, the present invention provides a method for preventing, treating or ameliorating an ErbB receptor related diseases or disorder, comprising administering to a subject in need thereof a pharmaceutical composition comprising as an active ingredient a polynucleotide comprising the sequence set forth in any one of SEQ ID NOS: 128 to 139 and SEQ ID NOS: 148, 150-159, 164-176.

According to yet another embodiment, the ErbB receptor related diseases or disorders are selected from the group consisting of neoplastic disease, hyperproliferative disorders, angiogenesis, restenosis, wound healing, psychiatric disorders, neurological disorders and neural injury.

As it is anticipated that at least some of the novel ErbB splice variants having a truncated EGF domain lacking the C-loop of the intact EGF domain, may act as antagonists rather than agonists it is to be understood that these variants will be useful to prevent or diminish any pathological response mediated by a ligand agonist. Thus, the neoplastic, hyperproliferative, angiogenic or other response may be attenuated or even abrogated by exposure or treatment with an antagonist according to the present invention.

Furthermore, if an agonist ligand predisposes stem cells to proliferate, survive, migrate, enter or commit to a specific lineage, then exposure or treatment with an antagonist would have the potential to alter the lineage commitment or differentiation pattern, or enhance proliferation prior to commitment to a given cell lineage. According to yet further aspects the present invention provides methods for selectively modulating the survival, proliferation, migration or differentiation of stem cells expressing ErbB receptors, comprising exposing the stem cells to an ErbB ligand splice variant, according to the present invention. Preferably, said stem cells are of neural, cardiac or pancreatic lineages, as ErbB ligands are known in the art to be involved in the development of these lineages.

According to one embodiment, the present invention provides a method for selectively modulating the survival, proliferation, migration or differentiation of stem cells expressing ErbB receptors, comprising exposing the stem cells to an ErbB ligand splice variant comprising the amino acid sequence set forth in any one of SEQ ID NOS:73 to 84 and 93, 95-104, 109-121. More preferably said stem cells are selected from neural, cardiac or pancreatic stem cell lineages.

According to further aspects the present invention provides methods of inhibiting the expression of the ErbB ligand splice variant by targeting the expressed transcript of such splice variant using antisense hybridization, small inhibitory (siRNA) or microRNA inhibition and ribozyme targeting.

The present invention is explained in greater detail in the description, figures and claims that follow.

### BRIEF DESCRIPTION OF THE FIGURES

FIG. 1 Depicts multiple sequence alignment of the evolutionarily conserved EGF domains for different known ErbB-ligands identified for worms (C. elegans), insects (Drosophila melanogaster) and mammals (humans or mice). Sequences shaded in grey demonstrate invariant residues in this alignment. Six cysteine residues are thought to be required for the formation of three disulfide loops within the domain for all these known ligands. An invariant Glycine and Arginine residue, considered critical for high-affinity ligand-receptor binding (boxed region). This multiple sequence alignment was generated by ClustalX (version 1.81) with modification, using the following protocol: The mammalian sequences were independently aligned by ClustalX (default parameters). This was repeated for the invertebrate ligands. These alignments were then treated as independent profiles, where the profile of mammalian sequences was aligned against the profile of invertebrate sequences, once again using clustalX (profile mode). All calculations were performed using default program parameters.

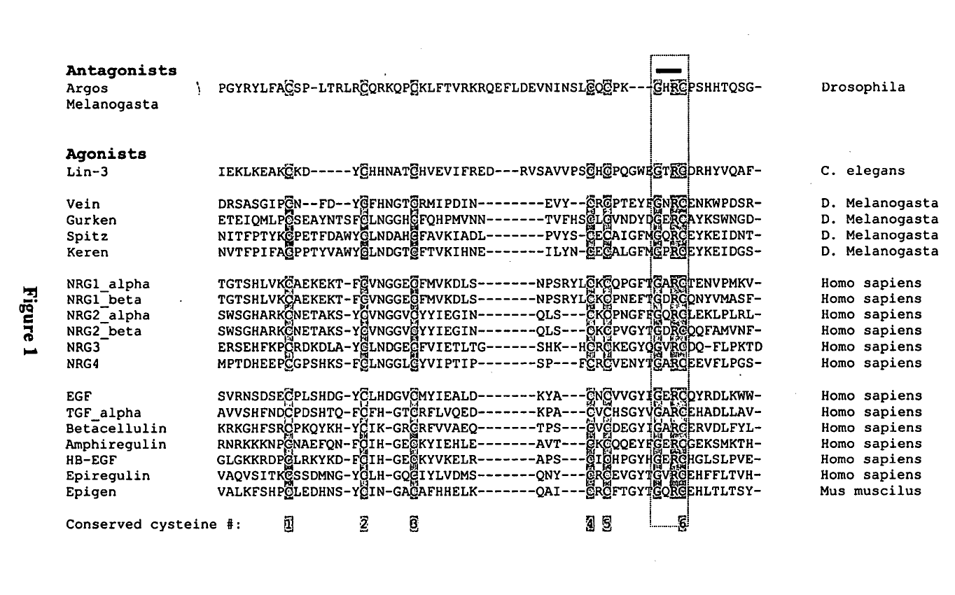

FIG. 2 Represents multiple sequence alignment of Argos primary protein sequences published for three independent insect species, Drosophila melanogaster, Drosophila virilis and Musca domestica. Two cysteine-rich domains defined as A1 and A2 and the EGF domain are marked in bold-set and underlined. The definitions demarking these domains have been borrowed from elsewhere (Howes et. al, 1999). Regions of highly conserved residues indicate the presence of critical domains within the Argos protein sequences. Similarly, the Musca domestica protein sequence demonstrates that an invariant Arg residue found in the EGF domain for all other receptor agonists (see FIG. 1) is not necessarily conserved in insect Argos (boxed region). * denotes invariant residues; : denotes conserved residues; . denotes Semi-conserved residues.

FIG. 3 Shows multiple sequence alignment of the receptor-modulating EGF domain encoded by different mammalian ErbB-ligands. Multiple sequence alignment of the receptor-binding EGF domain encoded by different mammalian ErbB-ligands were used as an input from which to generate a sequence profile in order to perform profile searches against various databases using a Compugen (hosted at EMBL) Bioccelerator. This alignment was generated by ClustalX version 1.81 and with minor manual modification. *=Invariant residues, :=Conserved residues, .=Semi-conserved residues.

FIG. 4 presents an examination of the genomic locus encoding “Exon A” of the EGF domain for the Neuregulin/EGF ligand family. The genomic sequence encoding Exon A for each ligand was extracted from the NCBI human (or where indicated mouse) genomic database. The genomic sequence was then translated, this including extended sequence running into and beyond the 5′ exon:intron splice junction which typically demarks the end of Exon A. This ‘extended Exon A’ potentially encodes an invariant in-frame stop codon positioned at precisely the same coordinate for all ErbB ligands relative to cysteine 4 of the EGF domain. The protein sequences of the full-length EGF domains are aligned in this figure against the translated sequence of extended Exon A. Exon A and Exon B are alternatively shaded. The presence of a stop codon is denoted by an asterisk (*). Dotted lines ( . . . ) indicate that the exon-encoding sequences extend beyond this alignment. The protein sequences present in this figure are listed herein as indicated (SEQ ID NOS:14-26, and 73-84). The nucleotide sequences encoding extended Exon A for each ligand are also provided (SEQ ID NOS:128-139). The EGF domain encoding full length mouse epigen is given here, as the human sequence was not available at the time of this analysis. The “extended exon A” sequence derived from genomic data are provided for both species.

FIG. 5 Demonstrates that genes encoding EGF domains other than ErbB-ligands display a heterogeneous intron-exon structure at the genomic level.

FIG. 5A shows a schematic diagram of the EGF domain structure for TGF alpha, EGF and Notch-1. The proteins TGF alpha, EGF and Notch-1 harbor one, nine and thirty-six EGF domains within their respective sequences as shown (diagram is not to scale). EGF domains are represented as boxes. The transmembrane domain of both EGF and TGF-alpha are represented as vertical black bars. Other unrelated domains are ignored in this diagram. The EGF domains responsible for receptor activation (for both EGF and TGF alpha) are denoted as shaded boxes followed by an astersik (*). Epidermal Growth Factor comprises an additional eight EGF domains not thought to directly activate the receptor. Notch-1 is not considered an ErbB ligand and is shown here as an example of an unrelated protein which also harbors EGF domains (unshaded boxes).

FIG. 5B provides an examination of the genomic locus encoding different EGF domains for human TGF alpha, EGF and Notch-1. The protein sequences for TGF alpha (i), EGF (ii) and Notch-1 (iii) were blasted against the human genomic database (tblastn; NCBI), to examine the exon structure for these genes. The EGF domains of these protein sequences were identified using the SMART database with manual adjustment, where flanking sequences have been ignored. These domain sequences were aligned (Clustalx version 1.81; standard parameters). Dark and light shading indicate the genomic topology demarking exon-exon boundaries within a particular EGF domain. The coordinates of each EGF domain is given in each case. For example, the first EGF domain which spans amino acids 24-57 for Notch-1 is shown as EGF_{—}24_{—}57. The protein sequences and genomic sequences used to examine TGF alpha, EGF and Notch-1 were derived from the NCBI accessions [P01135, NT_{—}022184.9], [NP_{—}001954.1, NT_{—}028147.9] and [AAG33848, NT_{—}024000.13] respectively. Of the aligned domains, the exceptional examples of ErbB-receptor-activating EGF domains are typed in bold-set and demarked with an asterisk (*). Of the forty four EGF domains examined which do not directly activate ErbB receptors (thirty six domains for Notch-1 and eight domains for EGF), only two of these (Notch-1 EGF domains number 1 and 30) harbor an exon-exon boundary which splits Cysteine 1-4 and Cys 5-6. The first EGF domain of Notch-1 is not fully shaded, due to the lack of this segment of genomic sequence found in the BLAST alignment.

FIG. 6 shows the Biocore binding profiles for mEGF(1-32) & hNRG2(1-32) against immobilized betacellulin. mEGF(1-32) and hEGF(1-32) at the indicated concentrations were injected over the surface of a Biacore chip with immobilized betacellulin and the resulting sensor curves were subtracted against a blank channel to yield the specific responses indicated. The results indicate low affinity interaction between each of the two peptides shown with Betacellulin. (RU—Resonance Unit)

### DETAILED DESCRIPTION OF THE INVENTION

The present invention is directed to (i) novel ErbB ligand isoforms identified as splice variants of at least one known ErbB ligand; (ii) polynucleotide sequences encoding the novel splice variants; (iii) oligonucleotides and oligonucleotide analogs derived from said polynucleotide sequences; (v) antibodies recognizing said splice variants; (vi) peptides or peptide analogs derived from said splice variants; and (vii) pharmaceutical compositions; and (viii) methods of employing said polypeptides, peptides or peptide analogs, said oligonucleotides and oligonucleotide analogs, and/or said polynucleotide sequences to regulate at least one ErbB receptor mediated activity.

While conceiving the present invention it was hypothesized that additional, previously unknown, ErbB ligands may exist. Splice variants, which occur in over 50% of human genes, are usually overlooked in attempts to identify differentially expressed genes, as their unique sequence features including donor-acceptor concatenation, an alternative exon, an exon and a retained intron, complicate their identification. However, splice variants may have an important impact on the understanding of disease development and may serve as valuable markers in various pathologies.

ErbB Ligand Splice Variants

The exact definition of what may constitute the boundaries of an ErbB-ligand receptor activating EGF domain is a matter of dispute. A conservative and limiting view is that it spans Cysteine 1 to Cysteine 6 (C1-C6) precisely (e.g. Howes et al. 1998). Even smaller sub-domains of this region were reported to weakly bind to receptors and to induce low levels of biological activity (reviewed in Groenen et al. 1994). An alternative definition is based upon the natural cleavage pattern of pro-ligands, in which EGF-domain containing peptides of varying length are generated after proximal and distal cleavage events (Harris et al. 2003). Yet other definitions rely upon biochemical and bioactivity analyses of synthetic and recombinant peptides of varying length, to reconstitute “typical” ligand function. From such analyses, it is apparent that additional carboxy and amino terminal sequences flanking C1-C6 are required to reconstitute ligand function. The exact length required for “typical” function may differ from ligand to ligand, as has been experimentally demonstrated in studies based upon binding and bioactivity assays (Barbacci et al. 1995; Groenen et al. 1994; Jones et al. 1999). Even so, it is evident that such definitions may vary depending on the biological assay performed. For example, biological assays based upon elucidation of receptor-binding affinity for a synthetic ligand peptide alone may demonstrate that a particular ligand of defined length binds very weakly. However, potent mitogenic low affinity ligands have been described in nature (for example Tzahar et al. 1998). Thus a disparity exists between these two biological parameters.

Although each Neuregulin gene encodes only a single EGF domain, both NRG-1 and NRG-2 genes comprise splice variants in which the carboxy-terminus of the EGF domain can be encoded by two alternative exons (the resultant variants termed alpha and beta). These alternatively encoded ligands possess different binding affinities and capacities to heterodimerize with the four different ErbB receptors (reviewed by Falls, 2003).

The ability to generate alpha and beta isoforms for NRG1 and NRG2 are reflected at the genomic level, where the carboxyl terminus of the EGF domain is encoded by alternate exons. More specifically, for both NRG1 and NRG2, a single exon encodes the amino-terminal component of the EGF domain, spanning C1-C4 and constituting the A-loop and B-loop of the EGF domain. An alternative choice of exons encode the remainder of the domain, which harbors C5-C6; the C-loop of the EGF domain (Crovello et al. 1998). Interestingly, all other members of the ErbB ligand family also share a similar segmented exon domain structure, precisely encoding C1-C4 and C5-C6 of the receptor-activating EGF domains on adjacent exons. However, for all these ligands other than NRG1 and NRG2, there has been no evidence to indicate that they encode alpha and beta alternative isoforms of the EGF domain, thus the evolutionary forces which are maintaining these conserved exon-exon topologies at the genomic level remains enigmatic (Harris et al. 2003; D. Harari, BigRock Seminar, the Weizmann Institute of Science, Feb. 5^{th}, 2001). The functional significance of the maintenance of this exon-exon structure of the receptor-activating EGF domains has remained unresolved, and is the major impetus for the present invention.

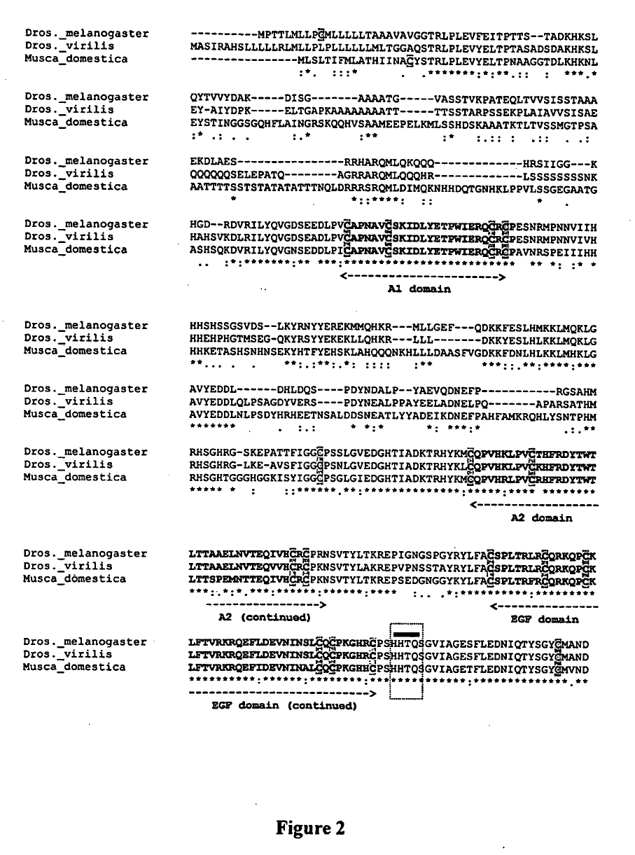

To date only one ErbB ligand having antagonist activity has been identified, namely the Argos ligand from insects. The Argos EGF domain is essential for this ligand's inhibitory function (Howes et. al, 1998). However, the mechanism in which Argos functions as an inhibitory ligand is a matter of dispute. For example, one model suggests that Argos binds to the insect EGF Receptor directly, inhibiting the binding of agonist ligands (such as Spitz) and inhibiting receptor dimerization (Jin et. al. 2000), An alternative model suggests however, that Argos binds directly to agonist ligands (such as Spitz), sequestering the agonist from activating the receptor (Mark Lemmon; The Fourth Dubrovnik Signaling Conference, FEBS, May 2004). The major objective of the present invention is to identify additional ErbB ligands that may possess inhibitory activity, especially naturally occurring ligands, preferably from vertebrate species, more preferably from mammalian species, most preferably from humans. Besides the importance of the EGF domain, Drosophila Argos comprises two additional cysteine rich regions, which have been defined as A1 and A2 (Howes et al. 1998). The multiple sequence alignment of Argos from three species demonstrates that as for the EGF domain, domains A1 and A2 and adjacent sequences are highly conserved (FIG. 2), supporting an important physiological function of these domains in the function of the protein. This multiple alignment also demonstrates conservation of sequence for the EGF domain and flanking carboxyl-terminal sequence (FIG. 2).

Before describing the present proteins, nucleotide sequences, the compositions comprising same and methods of use thereof, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies, which are reported in the publications which might be used in connection with the invention. Nothing herein is to be construed as an admission that the invention is not entitled to antedate such disclosure by virtue of prior invention.

### DEFINITIONS

ErbB ligand, as used herein, refers to the amino acid sequences of substantially purified ErbB ligand obtained from any species, particularly higher vertebrates, especially mammalian, including bovine, ovine, porcine, murine, equine, and preferably human, from any source whether natural, synthetic, semi-synthetic, or recombinant.

As used herein in the specification and in the claims that follow, the phrase “complementary polynucleotide sequence” includes sequences which originally result from reverse transcription of messenger RNA using a reverse transcriptase or any other RNA dependent DNA polymerase. Such sequences can be subsequently amplified in vivo or in vitro using a DNA dependent DNA polymerase.

As used herein in the specification and in the claims section that follows, the phrase “genomic polynucleotide sequence” includes sequences which originally derive from a chromosome and reflect a contiguous portion of a chromosome.

As used herein in the specification and in the claims section that follows, the phrase “composite polynucleotide sequence” includes sequences which are at least partially complementary and at least partially genomic. A composite sequence can include some exonal sequences required to encode a polypeptide, as well as some intronic sequences interposing therebetween. The intronic sequences can be of any source, including of other genes, and typically will include conserved splicing signal sequences. Such intronic sequences may further include cis acting expression regulatory elements.

As used herein in the specification and in the claims the phrase “splice variants” refers to naturally occurring nucleic acid sequences and proteins encoded therefrom which are products of alternative splicing. Alternative splicing refers to intron inclusion, exon exclusion, alternative exon usage or any addition or deletion of terminal sequences, which results in sequence dissimilarities between the splice variant sequence and other wild-type sequence(s). Although most alternatively spliced variants result from alternative exon usage, some result from the retention of introns not spliced-out in the intermediate stage of RNA transcript processing.

An “allele” or “allelic sequence”, as used herein, is an alternative form of the gene encoding an ErbB ligand. Alleles may result from at least one mutation in the nucleic acid sequence and may result in altered mRNAs or polypeptides whose structure or function may or may not be altered. Any given natural or recombinant gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

“Altered” nucleic acid sequences encoding an ErbB ligand as used herein include those with deletions, insertions, or substitutions of different nucleotides resulting in a polynucleotide that encodes the same or a functionally equivalent ErbB ligand. Included within this definition are polymorphisms which may or may not be readily detectable using a particular oligonucleotide probe of the polynucleotide encoding a particular ErbB ligand, and improper or unexpected hybridization to alleles, with a locus other than the normal chromosomal locus for the polynucleotide sequence encoding the ErbB ligand. The encoded protein may also be “altered” and contain deletions, insertions, or substitutions of amino acid residues which produce a silent change and result in a functionally equivalent ErbB ligand. Deliberate amino acid substitutions may be made on the basis of similarity in polarity, charge, solubility, hydrophobicity, hydrophilicity, and/or the amphipathic nature of the residues as long as the biological or immunological activity of the ErbB ligand is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

“Amino acid sequence”, as used herein, refers to an oligopeptide, peptide, polypeptide, or protein sequence, and fragment thereof, and to naturally occurring or synthetic molecules. Fragments of ErbB ligands are preferably about twenty to about forty amino acids in length and retain the biological activity or the immunological activity of the intact ligand. Where “amino acid sequence” is recited herein to refer to an amino acid sequence of a naturally occurring protein molecule, amino acid sequence, and like terms, are not meant to limit the amino acid sequence to the complete, native amino acid sequence associated with the recited protein molecule.

“Amplification” as used herein refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.).

The term “activatory ligand” or “agonist”, as used herein, refer to a ligand which upon binding stimulates ErbB signaling in a receptor-dependent manner. Without contradiction, under certain circumstances, a ligand may be correctly described either as activatory and inhibitory, depending on the environmental and experimental context in which it has been described.

The term “inhibitory ligand” or “antagonist”, as used herein interchangeably, refers to a molecule which decreases the amount or the duration of the effect of the biological or immunological activity of a known ligand to an ErbB receptor. The antagonist may function by directly or indirectly binding to an ErbB receptor. The antagonist may additionally or separately function by another mechanism however, in which the antagonist will directly or indirectly bind to an activatory ErbB ligand, thus sequestering it from receptor-dependent activation.

The term “inhibitor” refers to a molecule or compound that that exerts an inhibitory effect on the function of the ErbB ligand splice variant of the present invention. The inhibitor may include proteins, peptides, nucleic acids, antibodies or any other molecules which decrease the effect of the variant ErbB ligand.

As used herein, the term “antibody” refers to intact molecules as well as fragments thereof, such as Fab, F(ab′)₂, and Fv, which are capable of binding the epitopic determinant. Antibodies that bind ErbB ligand polypeptides can be prepared using intact polypeptides or fragments containing small peptides of interest as the immunizing antigen. The polypeptide or oligopeptide used to immunize an animal can be derived from the translation of RNA or synthesized chemically and can be conjugated to a carrier protein, if desired. Commonly used carriers that are chemically coupled to peptides include bovine serum albumin and thyroglobulin, keyhole limpet hemocyanin. The coupled peptide is then used to immunize the animal (e.g., a mouse, a rat, or a rabbit).

The term “antigenic determinant”, as used herein, refers to that fragment of a molecule (i.e., an epitope) that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The term “antisense”, as used herein, refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

The term “biologically active”, as used herein, refers to a protein having structural, regulatory, or biochemical functions of a naturally occurring molecule. Likewise, “immunologically active” refers to the capability of the natural, recombinant, or synthetic ErbB ligand, or any oligopeptide thereof, to induce a specific immune response in appropriate animals or cells and to bind with specific antibodies.

The term “active fragment” refers to any variant with the truncated domain lacking the C-loop as the minimal receptor modulating fragment. An active fragment may be defined as any fragment having less than the six conserved cysteines of the intact EGF domain capable of perturbing the activity of at least one ErbB receptor subtype. Preferably the term active fragment refers to any fragment having less than the six conserved cysteines of the intact EGF domain capable of perturbing the activity of at least one ErbB receptor subtype, further comprising flanking amino acid sequences known to increase the receptor binding and/or ligand induced receptor mediated activity.

The terms “complementary” or “complementarity”, as used herein, refer to the natural binding of polynucleotides under permissive salt and temperature conditions by base-pairing. For example, the sequence “A-G-T” binds to the complementary sequence “A-C-T”

Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of peptide nucleic acid (PNA) molecules.

A “composition comprising a given polynucleotide sequence” as used herein refers broadly to any composition containing the given polynucleotide sequence. The composition may comprise a dry formulation or an aqueous solution. Compositions comprising polynucleotide sequences encoding a novel ErbB ligand splice variant according to the present invention, or specific fragments thereof may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS) and other components (e.g., Denhardt's solution, dry milk, salmon sperm DNA, etc.).

A “deletion”, as used herein, refers to a change in the amino acid or nucleotide sequence and results in the absence of one or more amino acid residues or nucleotides.

The term “derivative”, as used herein, refers to the chemical modification of a nucleic acid encoding or complementary to an ErbB ligand or to the chemical modification of the encoded ErbB ligand. Such modifications include, for example, replacement of hydrogen by an alkyl, acyl, or amino group. A nucleic acid derivative encodes a polypeptide which retains the biological or immunological function of the natural molecule. A derivative polypeptide is one which is modified by glycosylation, pegylation, or any similar process which retains the biological or immunological function of the polypeptide from which it was derived.

The term “homology”, as used herein, refers to a degree of sequence similarity in terms of shared amino acid or nucleotide sequences. There may be partial homology or complete homology (i.e., identity). For amino acid sequence homology amino acid similarity matrices (e.g. BLOSUM62, PAM70) may be utilized in different bioinformatics programs (e.g. BLAST, FASTA, Smith Waterman). Different results may be obtained when performing a particular search with a different matrix or with a different program. Degrees of homology for nucleotide sequences are based upon identity matches with penalties made for gaps or insertions required to optimize the alignment, as is well known in the art.

The term “humanized antibody”, as used herein, refers to antibody molecules in which amino acids have been replaced in the non-antigen binding regions in order to more closely resemble a human antibody, while still retaining the original binding ability.

The term “hybridization”, as used herein, refers to any process by which a strand of nucleic acid binds with a complementary strand through base pairing.

An “insertion” or “addition”, as used herein, refers to a change in an amino acid or nucleotide sequence resulting in the addition of one or more amino acid residues or nucleotides, respectively, as compared to the naturally occurring molecule.

“Microarray” refers to an array of distinct polynucleotides or oligonucleotides synthesized on a substrate, such as paper, nylon or other type of membrane, filter, chip, glass slide, or any other suitable solid support.

The term “modulate”, as used herein, refers to a change in the activity of at least one ErbB receptor mediated activity. For example, modulation may cause an increase or a decrease in protein activity, receptor binding characteristics, ligand sequestration, or any other biological, functional or immunological properties of an ErbB ligand.

“Nucleic acid sequence” as used herein refers to an oligonucleotide, nucleotide, or polynucleotide, and fragments thereof, and to DNA or RNA of genomic or synthetic origin which may be single- or double-stranded, and represent the sense or antisense strand. “Fragments” are those nucleic acid sequences which are greater than 60 nucleotides than in length, and most preferably includes fragments that are at least 100 nucleotides in length.

The term “oligonucleotide” refers to a nucleic acid sequence of at least about 6 nucleotides to about 60 nucleotides, preferably about 15 to 30 nucleotides, and more preferably about 20 to 25 nucleotides, which can be used in PCR amplification or a hybridization assay, or a microarray. As used herein, oligonucleotide is substantially equivalent to the terms “amplimers”, “primers”, “oligomers”, and “probes”, as commonly defined in the art.

The term “peptide nucleic acid” (PNA) as used herein refers to nucleic acid “mimics”; the molecule's natural backbone is replaced by a pseudopeptide backbone and only the four-nucleotide bases are retained. The peptide backbone ends in lysine, which confers solubility to the composition. PNAs may be pegylated to extend their lifespan in the cell where they preferentially bind complementary single stranded DNA and RNA and stop transcript elongation (Nielsen, P. E. et al. (1993) Anticancer Drug Des. 8:53-63).

The term “portion”, as used herein, with regard to a protein (as in “a portion of a given protein”) refers to fragments of that protein. The fragments may range in size from five amino acid residues to the entire amino acid sequence minus one amino acid. Thus, a protein “comprising at least a portion of the amino acid sequence of SEQ ID NO:1” encompasses the full-length PNIN and fragments thereof.

The term “sample”, as used herein, is used in its broadest sense. A biological sample suspected of containing nucleic acid encoding an ErbB ligand, or fragments thereof, or the encoded polypeptide itself may comprise a bodily fluid, extract from a cell, chromosome, organelle, or membrane isolated from a cell, a cell, genomic DNA, RNA, or cDNA in solution or bound to a solid support, a tissue, a tissue print, and the like.

The terms “specific binding” or “specifically binding”, as used herein, refers to that interaction between a protein or peptide and an agonist, an antibody and an antagonist. The interaction is dependent upon the presence of a particular structure (i.e., the antigenic determinant or epitope) of the protein recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

The terms “stringent conditions” or “stringency”, as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature of the probe to about 20° C. to 25° C. below the melting temperature). One or more factors be may be varied to generate conditions of either low or high stringency different from, but equivalent to, the above listed conditions.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

A “substitution”, as used herein, refers to the replacement of one or more amino acids or nucleotides by different amino acids or nucleotides, respectively.

“Transformation”, as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

A “variant” of an ErbB ligand, as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR software.

A “splice variant” of an ErbB ligand as used herein and in the claims refers to any variant of the known ErbB ligands, including truncation variants, deletion variants, alternative exon usage, and intronic sequences, that each comprise at least one altered component of the EGF domain that affects ligand-mediated ErbB receptor activation. Specifically the term splice variant includes all such variants that lack the C5-C6 loop of the corresponding known EGF domain.

### Novel Inhibitory Ligands Identified by a Bioinformatics Approach:

Utilizing a methodology of sequence comparison, it has been possible to identify homologous ErbB ligand agonists by a bioinformatics approach (e.g. (Harari et al. 1999)). However, despite the wealth of sequence data that is publicly available, no naturally known mammalian inhibitory ErbB ligand has been described in the literature to date. Indeed a preliminary BLAST-based database search failed to identify mammalian genes with sequences sufficiently similar that of insect Argos-like proteins to be readily identified (data not shown).

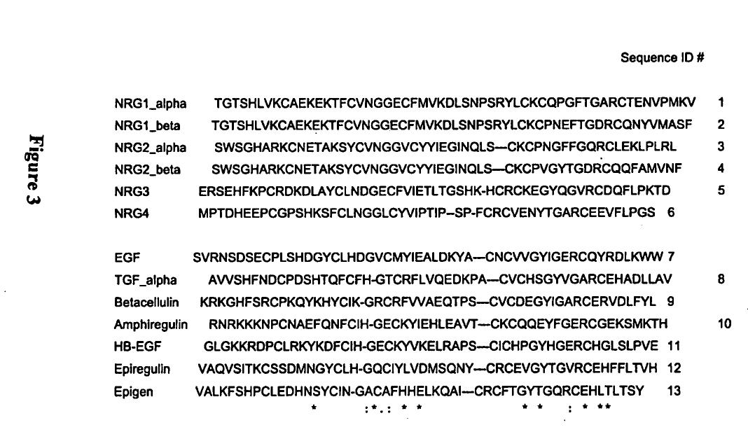

Thus, it was decided to perform searches for sequences that may harbor EGF-like domains with a profile somewhat typical to that already known for members of the mammalian ErbB-ligand family. It should be noted that this search is biased to the identification of ligand agonists, as all known mammalian ligands to date are agonists. However, if the EGF domain of mammalian ErbB antagonist ligands are sufficiently similar to that of their agonist counterparts, it may be possible to identify them by sequence similarity search. Protein sequences for different mammalian ligands were therefore retrieved from the NCBI server (see Tables 5 and 6). Approximate identification of the coordinates in which the receptor-modulating EGF domains for each ligand was revealed and defined by the SMART server. These domains were arbitrarily lengthened to provide a greater span of amino and carboxyl sequences which may be helpful for the identification of novel ligands, and were subsequently aligned using ClustalX. Minor modification to the sequence alignments were performed manually (FIG. 3).

This multiple sequence alignment was subsequently used to create a profile using the program PROFILEWEIGHT (see materials and methods). Translated profile searches were then performed against the EST databases provided at the EMBL site (see materials and methods). At the time of these searches, the EST database was split into five partitions at the EMBL site and each partition was independently scanned by TPROFILESEARH. These searches were performed using global alignments and the choice of gap opening penalties and gap extension penalties (GOP & GEP) being set at (10 & 1) or (12 & 1) respectively with a predefined output of 500 sequences to be aligned per search. No novel ESTs with an obvious encoded sequence profile similar to that typical to the EGF domain of ErbB ligands were identified.

Since it has already been observed that the exon organization encoding all mammalian ErbB ligands at the site of the EGF domain is conserved, it was decided to explore the possibility that alternative ErbB splice variants encoding partial, alternative or truncated EGF domains may be expressed. For example, a truncated form of NRG1, encoding a partial EGF domain up to cysteine 4, followed by a stop codon has been reported (Falls 2003). Splice isoforms can be better characterized when the variants are examined in the context of the genomic sequence encoding each gene.

It was thus decided to extract co-currently the genomic sequences encoding the mammalian ErbB ligands. As a matter of convenience, nomenclature is provided herein to better describe the exons that typically encode the receptor-modulating EGF domain for the mammalian ErbB ligands. The first exon encoding the first component of the EGF modulating domain of ErbB ligands (including C1-C4) is described herein as “Exon A” of the EGF domain. The second exon encoding the second component of the EGF domain (including C5-C6) is described herein as “Exon B” of the EGF domain. In the case of NRG1 and NRG2. which harbor alternative (alpha and beta) carboxyl isoforms of the EGF domain, these are considered herein as exon B (for alpha isoforms) or exon B′ (for beta isoforms) of the EGF domain. Genomic sequences encoding the different mammalian ErbB ligands were extracted from the NCBI database (See Tables 5 and 6). For each gene, the genomic region encoding Exon A including flanking sequences, was identified and translated (using Transeq).

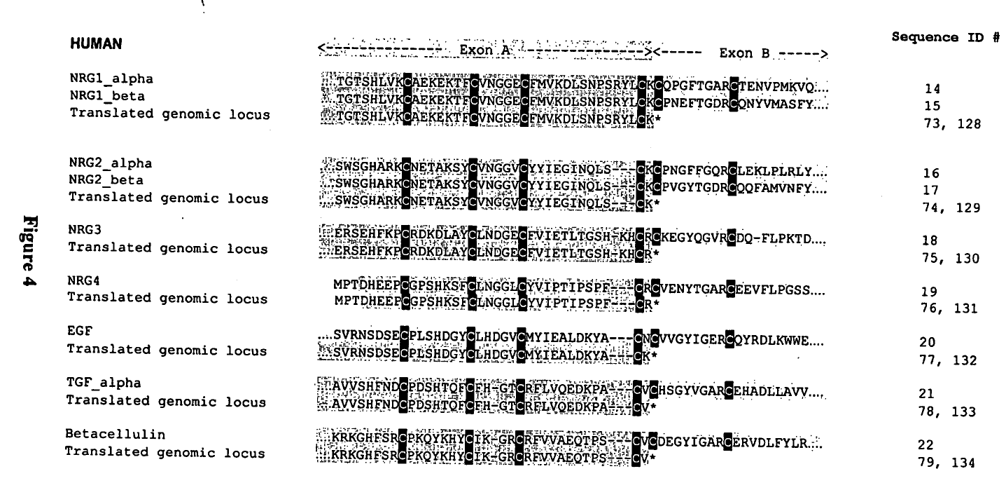

A surprising result was observed. Not only is the position of the exon-exon junction for Exon A and Exon B conserved for all mammalian ErbB ligands, in what would typically be considered as “intronic” region just beyond Exon A, an invariant stop codon has been found and is encoded both in-frame and immediately downstream of Exon A (FIG. 4). This provides indirect evidence to support that alternative isoforms of all mammalian ligands may exist in which the encoded proteins harbor truncated EGF domains. Specifically, such splice variants would encode the EGF domain to one amino acid beyond Cysteine 4 (FIG. 4) as a result of the extension in length of exon A of the EGF domain. Similar topology was found for genes encoding other mouse ErbB ligands and where available other vertebrate species, including for example bovine and chicken, indicating that the observations observed with the human sequences herein are shared by mammals, birds and other higher vertebrates (data not shown).

An examination of the expanded exon A nucleotide sequence (SEQ ID NOS:128-139) demonstrates that for each ligand a common consensus pattern leading to the termination of the translation product. The sequences harbor the consensus G,TXX, where the comma denotes the codon reading frame and TXX encodes a stop codon. The di-nucleotide motif “GT” is required to maintain the evolutionarily conserved exon:intron splice junction that is observed at this site (Darnell et. al. 1986).

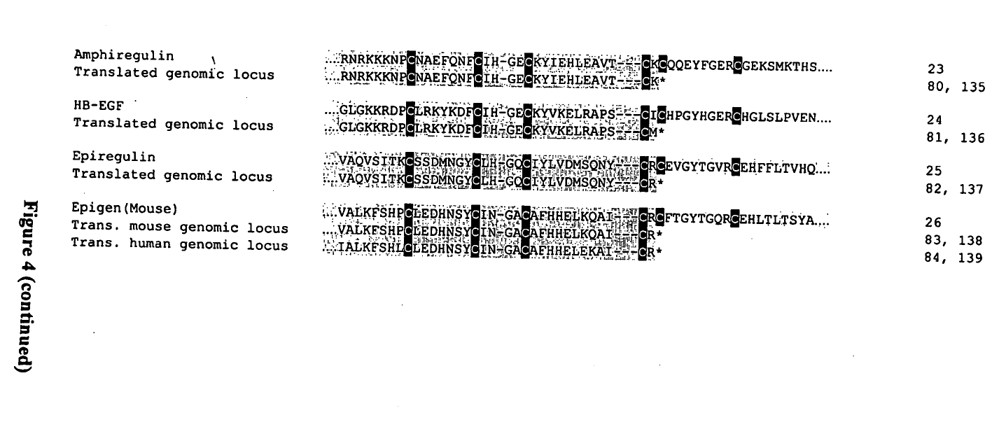

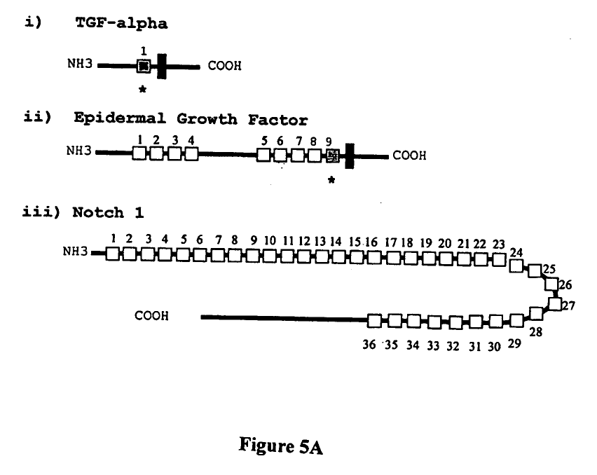

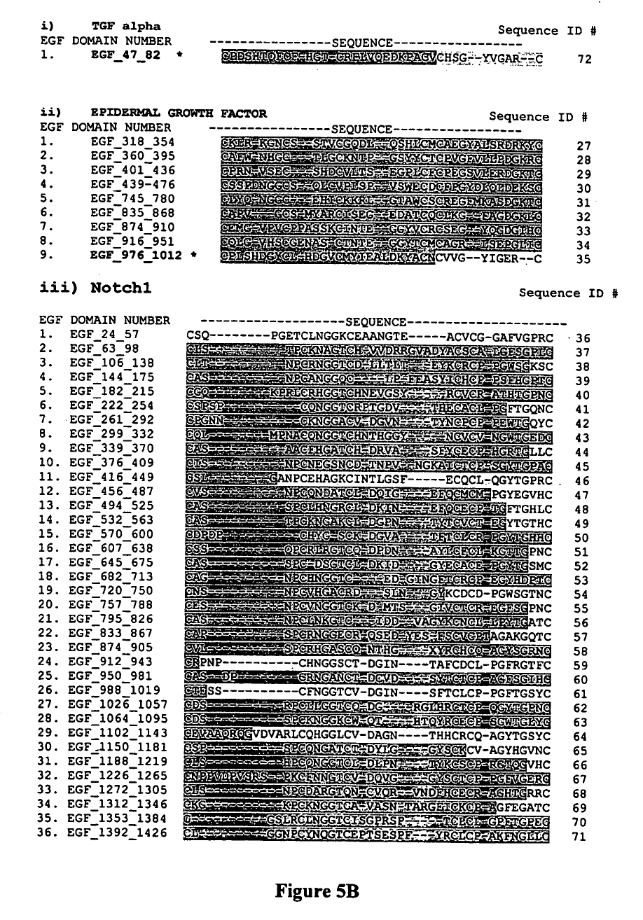

Thus, an initial hypothesis is provided that the evolutionarily conserved genomic topology of the EGF domain is preserved in order to allow the generation of ErbB-ligand splice variants which are truncated after cysteine-4 of the EGF domain. A negative hypothesis to this concept, is that the exon-exon structure encoding the mammalian ErbB ligand receptor-modulating EGF domains has nothing to do with the formation of splice variants, but rather is a result of the general genomic topology found for EGF domain sequences (for reasons that may be known or unknown). EGF domains are commonly encoded by many proteins, with functions that in the most part are unrelated to ErbB-ligand activation (Carpenter and Cohen 1990). Thus it was tested if the invariant genomic organization found for the receptor-modulating EGF domains for the ErbB ligands is also preserved in genomic sequences encoding a sample of unrelated EGF domains. To test this hypothesis, the proteins TGF alpha (as a reference), Epidermal Growth Factor and Notch-1 were tested. TGF alpha harbors a single EGF domain, which is responsible for receptor binding and activation. The Epidermal Growth Factor in comparison comprises nine EGF domains; only the ninth of these being responsible for receptor binding and activation. Notch-1 conversely is another signaling molecule that harbors thirty six EGF domains, none of these being responsible for ErbB-receptor activation (FIG. 5A). The genomic sequences encoding these three genes were examined, in order to elucidate the genomic organization encoding their different EGF domains. For the epidermal growth factor, only the ErbB-receptor-binding EGF domain was encoded by a split codon. In contrast, the eight remaining EGF domains were wholly encoded within individual exons (FIG. 5B). Conversely, for Notch-1, a heterogeneous genomic organization was observed for the thirty six encoded EGF domains (FIG. 5B). Of these, only the first and the thirtieth EGF domain harbors a split exon topology at the position found for the ErbB-receptor binding domains. From these data it can be concluded that the general topology of genomic DNA encoding EGF domains in general does not necessarily require a split exon-exon structure and stop codon encoded immediately after Exon A, as demonstrated for the ErbB-receptor binding domains in mammals.

Genes encoding ErbB ligands that do not harbor a split exon-exon structure encoding the EGF domain remain biologically active. For example, virally encoded ErbB ligands exist in nature, even though their genomes lack intronic sequences to split the EGF domain encoding region (E.g. VGF; NCBI Accession number U18337, embedded protein sequence # AAA69306). Furthermore, it is common practice in molecular biology to express genes in the form of intron-less cDNA sequences under the control of various transcriptional promoters (Maniatis et al. 1982). In this way recombinant genes encoding promoter-less ErbB ligands have been constructed, these which encode functional and active recombinant proteins (Groenen et al. 1994). Thus the evolutionary conserved exon-exon junctions found in genes encoding the different mammalian ErbB-ligands (FIG. 5) are not required for the generation of functional ligands harboring the conserved six-cysteine EGF domain in mammalian cells.

The formation of functional alternative splice variants of ErbB ligands with a shortened EGF domain that ends after cysteine 4 would provide a functional explanation as to the conservation of this domain sequence. The best proof that such truncated ErbB ligand variants exist in nature is to demonstrate that such isoforms are indeed expressed. A saturation cloning effort has been performed to pull out all isoforms of the well characterized NRG1 gene. Indeed there exists a truncated NRG1 variant, which is identical to other typical NRG1 alpha isoforms, except that its sequence ends one amino acid after the fourth cysteine of the EGF domain (Heregulin gamma—not to be confused with gamma heregulin (Falls 2003). An examination of this protein's encoding sequence (Accession numbers NP_{—}004486 and NM_{—}004495) in relation to the NRG1 genomic locus, furthermore confirms that this variant sequence harbors an extended exon A, resulting in it protein's truncation (data not shown). Therefore a proof of principle that such truncated variants exist is demonstrated for NRG1.

Randomly generated transcripts provide a very poor representation of ErbB ligand sequences in public databases, such as is the case for EST sequences, particularly due to the very low expression commonly found for these genes. Nevertheless a bioinformatics search was performed to search for expressed transcripts of genes, or gene fragments, in search of truncated ErbB ligands within the EGF domain. To achieve this, the EGF domain for the different mammalian ErbB ligands (FIG. 4) were used to query the NCBI NR, EST and PATENT genomic databases by method of TBLASTN, in order to search for sequences with truncated homologous sequences. These DNA sequences were extracted, and where appropriate translated into six reading frames (EMBOSS-Transeq). The relevant reading frame encoding the truncated EGF domain was chosen. Interestingly, two different classes of predicted protein sequences were discovered:

Class I: Sequences encoding a protein truncated after cysteine-4 as would be expected upon the extension of Exon A.

Class II: Sequences which encode a partial EGF domain (exon A) with alternative splice variations, in which Exon B is not encoded. The proteins encoded by this class of splice variant tends to be heterogeneous in length beyond the expression of the shortened EGF domain, depending on the alternative exon sequences that are present beyond exon A.

A list of the Class I and Class II protein sequences are shown below, inclusive of their encoded protein sequences. Unless the protein sequences were already known, the sequences provided here were translated and the appropriate reading frame encoding the truncated EGF domain was chosen. It should be noted that some of these sequences, particularly the EST sequences are partial sequences, and also are prone to occasional sequencing error. Thus, the full translated sequences are often given, regardless if an initiating methionine were noted in the translated sequence or not. These data verify the existence of two classes of ErbB ligand splice variants which encode a truncated EGF domain lacking the C-loop of the EGF domain, in a diverse range of species, including humans and other mammals, birds and fish.

TABLE 2
Class I variants

| Nucleotide | | | | | Linked Protein |
|---|---|---|---|---|---|
| Sequence | | Accession | Database & | | Sequence |
| ID No. | Gene | Number | Details | Species | ID No. |
| 140 | NRG1 | A81177.1 | Patent | Human | 85 |
| | | | WO9914323 | | |
| 141 | NRG1 | AX269478.1 | Patent | Human | 86 |
| | | | WO0164876 | | |
| 142 | NRG1 | AX271009.1 | Patent | Human | 87 |
| | | | WO0164877 | | |
| 143 | NRG1 | NM_004495.1 | NR | Human | 88 |
| 144 | NRG1 | AF026146.1 | NR | Human | 89 |
| 145 | NRG1 | NM_178591.1 | NR | Mouse | 90 |
| 146 | NRG1 | AK051824.1 | NR (RIKEN) | Mouse | 91 |
| 147 | NRG1 | BY212704.1 | NR (RIKEN) | Mouse | 92 |
| 148 | NRG2 | AI041451.1 | EST | Human | 93 |
| 149 | NRG2 | AX406619.1 | Patent | Human | 94 |
| | | | WO0222685 | | |
| 150 | NRG3 | BX495970.1 | EST | Human | 95 |
| 151 | NRG4 | BE787057.1 | EST | Human | 96 |
| 152 | NRG4 | BF061527.1 | EST | Human | 97 |
| 153 | NRG4 | BX095400.1 | EST | Human | 98 |
| 154 | NRG4 | BB637399.1 | EST | Mouse | 99 |
| 155 | NRG4 | BB637505.1 | EST | Mouse | 100 |
| 156 | NRG4 | AI743118.1 | EST | Human | 101 |
| 157 | NRG4 | AU059620.1 | EST | Pig | 102 |
| 158 | NRG4 | C94578.1 | EST | Pig | 103 |
| 159 | TGF | AK089870.1 | NR (RIKEN) | Mouse | 104 |
| | alpha | | | | |
| 160 | TGF | I01190.1 | U.S. Pat. No. | Human | 105 |
| | alpha | | 4,742,003 | | |
| 161 | Epiregulin | AR019352.1 | U.S. Pat. No. | Human | 106 |
| | | | 5,783,417 | | |
| 162 | Epiregulin | AR019354.1 | U.S. Pat. No. | Human | 107 |
| | | | 5,783,417 | | |
| 163 | Epiregulin | AR019353.1 | U.S. Pat. No. | Mouse | 108 |
| | | | 5,783,417 | | |
| 164 | Epiregulin | BC035806.1 | EST (HTC) | Human | 109 |
| 165 | Epiregulin | BM561909.1 | EST | Human | 110 |
| | | | (AGENCOURT) | | |

Sequences found in the EST, NR and Patent (DNA) databases having sequences encoding ErbB ligand variants comprising an elongated Exon A, resulting in a protein sequence truncated after the conserved cysteine-4 of the EGF domain. The list includes genomic fragments and transcript data.

TABLE 3
Class II variants

| | | | | | Corresponding |
|---|---|---|---|---|---|
| Nucleotide | | | | | Protein |
| Sequence ID | | | Database & | | Sequence ID |
| Number | Gene | Accession | Details | Species | Number |
| 166 | NRG2 | AA706226.1 | EST | Human | 111 |
| 167 | NRG2 | BX089049.1 | EST | Human | 112 |
| 168 | NRG2 | AI152190.1 | EST | Mouse | 113 |
| 169 | NRG2 | AL918370.1 | EST | Zebrafish | 114 |
| 170 | NRG3 | BU465274.1 | EST | Chicken | 115 |
| 171 | NRG4 | BU372401.1 | EST | Chicken | 116 |
| 172 | NRG4 | BE624667.1 | EST | Mouse | 117 |
| 173 | Amphiregulin | BE064716.1 | EST | Human | 118 |
| 174 | Betacellulin | BG194271.1 | EST (RAGE) | Human | 119 |
| 175 | BY735030.1 | BY735030.1 | EST (RIKEN) | Mouse | 120 |
| 176 | HB-EGF | X89728.1 | NR | Cercopithecus | 121 |
| | | | | aethiops (African | |
| | | | | green monkey) | |
| 177 | Epigen | BD274363.1 | Patent JP | Human | 122 |
| | | | 2002530064- | | |
| | | | A/7. | | |
| 178 | Epigen | AX261946.1 | Patent | Human | 123 |
| | | | WO0172781 | | |
| 179 | Epigen | AX261991.1 | Patent | Human | 124 |
| | | | WO0172781 | | |
| 180 | Epigen | BD274361.1 | Patent JP | Human | 125 |
| | | | 2002530064- | | |
| | | | A/5. | | |
| 181 | Epigen | BD209747.1 | Patent JP | Human | 126 |
| | | | 2002512798- | | |
| | | | A/219 | | |
| 182 | Epigen | BD274362.1 | Patent JP | Human | 127 |
| | | | 2002530064- | | |
| | | | A/6. | | |

Sequences found in the EST, NR and Patent (DNA) databases potentially encode ErbB ligands which include Exon A but lack Exon B, resulting in the predicted expression of proteins of varying lengths extending beyond that of a shortened EGF domain (to the conserved Cys-4).

DNA sequences encoding truncated Class I variants (FIG. 4):
Sequence ID # 128
ACTGGGACAAGCCATCTTGTAAAATGTGCGGAGAAGGAGAAAACTTTCTGTGTGAATGGAGGGGAGTGCT
TCATGGTGAAAGACCTTTCAAACCCCTCGAGATACTTGTGCAAGTAA
Sequence ID # 129
TCCTGGTCGGGGCACGCCCGGAAGTGCAACGAGACAGCCAAGTCCTATTGCGTCAATGGAGGCGTCTGCT
ACTACATCGAGGGCATCAACCAGCTCTCCTGCAAGTAA
Sequence ID # 130
GAGCGATCCGAGCACTTCAAACCCTGCCGAGACAAGGACCTTGCATACTGTCTCAATGATGGCGAGTGCT
TTGTGATCGAAACCCTGACCGGATCCCATAAACACTGTCGGTAA
Sequence ID # 131
GATCACGAAGAGCCCTGTGGTCCCAGTCACAAGTCGTTTTGCCTGAATGGGGGGCTTTGTTATGTGATAC
CTACTATTCCCAGCCCATTTTGTAGGTGA
Sequence ID # 132
TCCGTAAGAAATAGTGACTCTGAATGTCCCCTGTCCCACGATGGGTACTGCCTCCATGATGGTGTGTGCA
TGTATATTGAAGCATTGGACAAGTATGCATGCAAGTAA
Sequence ID # 133
GCAGTGGTGTCCCATTTTAATGACTGCCCAGATTCCCACACTCAGTTCTGCTTCCATGGAACCTGCAGGT
TTTTGGTGCAGGAGGACAAGCCAGCATGTGTGTAA
Sequence ID # 134
AAGCGGAAAGGCCACTTCTCTAGGTGCCCCAAGCAATACAAGCATTACTGCATCAAAGGGAGATGCCGCT
TCGTGGTGGCCGAGCAGACGCCCTCCTGTGTGTAA
Sequence ID # 135
AGAAACAGAAAGAAGAAAAATCCATGTAATGCAGAATTTCAAAATTTCTGCATTCACGGAGAATGCAAAT
ATATAGAGCACCTGGAAGCAGTAACATGCAAGTAA
Sequence ID # 136
GGGCTAGGGAAGAAGAGGGACCCATGTCTTCGGAAATACAAGGACTTCTGCATCCATGGAGAATGCAAAT
ATGTGAAGGAGCTCCGGGCTCCCTCCTGCATGTAA
Sequence ID # 137
GTGGCTCAAGTGTCAATAACAAAGTGTAGCTCTGACATGAATGGCTATTGTTTGCATGGACAGTGCATCT
ATCTGGTGGACATGAGTCAAAACTACTGCAGGTAA
Sequence ID # 138
GTAGCTCTGAAGTTCTCTCATCCTTGTCTGGAAGACCATAATAGTTACTGCATTAATGGAGCATGTGCAT
TCCACCATGAGCTGAAGCAAGCCATTTGCAGGTAA
Sequence ID # 139
ATAGCCTTGAAGTTCTCACACCTTTGCCTGGAAGATCATAACAGTTACTGCATCAACGGTGCTTGTGCAT
TCCACCATGAGCTAGAGAAAGCCATCTGCAGGTAA

Summary of Sequences in this Patent

Sequences 1-72 refer to known polypeptide sequences which are described in FIGS. 3, 4 and 5, and do not include the claimed novel ErbB splice variants. Sequences 73-182, including the novel ErbB ligand splice variants are summarized in Table 4.

TABLE 4
A summary of sequences harboring or encoding ErbB ligand
variants that do not encode Exon B of the EGF domain.

| | | Amino Acid | |
|---|---|---|---|
| ErbB | | Sequences/Translated | DNA Sequence |
| Variant | Details | Sequences ID Nos. | ID Nos. |
| Class I | Sequences of FIG. 4 | 73-84 | 128-139 |
| Variants | | | |
| Class I | Sequences of Table 2 | 85-110 | 140-165 |
| Variants | | | |
| Class II | Sequences of Table 3 | 111-127 | 166-182 |
| Variants | | | |

### Novel Splice Variants of ErbB Ligands

Currently preferred embodiments according to the present invention include isolated polynucleotides selected from the following:

1. Polynucleotides encoding the extended EGF domain derived directly from genomic data (denoted herein as Class I): namely SEQ ID NOS: 128 to 139.
2. Polynucleotides encoding Class I variants or fragments of variants derived from the EST and NR databases (Table 2 excluding heregulin (NRG1) gamma variants): namely SEQ ID NOS: 148, 150-159, 164-165.
3. Polynucleotides encoding Class II variants of fragments of variants derived from the EST and NR databases (Table 3): namely SEQ ID NOS:166 to 176.

It is explicitly understood that all known sequences are excluded from the scope of the present invention. However, it is further explicitly to be understood that any novel uses of sequences previously disclosed as lacking this utility are encompassed within the present invention.

Currently preferred embodiments according to the present invention include polypeptides comprising the following:

1. Polypeptides comprising truncated EGF domain derived directly from genomic data (denoted herein Class I) namely SEQ ID NOS:73 to 84.
2. Class I variants or fragments of variants derived from the EST, NR and Patent databases (translation of Table 2 sequences from the NR and EST databases excluding NRG1 gamma variants) namely

### SEQ ID NOS:93, 95-104, 109-110.

3. Class II variants of fragments of variants derived from the EST and NR databases (translated sequences of Table 3) namely SEQ ID NOS:111 to 121.

It is explicitly understood that all known sequences are excluded from the scope of the present invention.

Thus, according to one aspect of the present invention there are provided isolated nucleic acids comprising a genomic, complementary or composite polynucleotide sequence encoding a polypeptide being capable of modulating a mammalian ErbB which is at least 70%, preferably at least 80%, more preferably at least 90% or more, say at least 95%, or 100% homologous (similar+identical acids) to SEQ ID NOS:73-84 and SEQ ID NOS:93, 95-104, 109-121. Homology is determined for example using Gapped BLAST-based searches (Altschul et. al. 1997) with preferred matrix BLOSUM62 (protein-based searches) and the following default parameters as defined by the NCBI BLAST web site:
−G Cost to open gap [Integer]
default=5 for nucleotides 11 proteins
−E Cost to extend gap [Integer]
default=2 nucleotides 1 proteins
−q Penalty for nucleotide mismatch [Integer]
default=−3
−r reward for nucleotide match [Integer]
default=1
−e expect value [Real]
default=10
−W wordsize [Integer]
default=11 nucleotides 3 proteins
−y Dropoff (X) for blast extensions in bits (default if zero)
default=20 for blastn 7 for other programs
−X X dropoff value for gapped alignment (in bits)
default=15 for al programs except for blastn for which it does not apply
−Z final X dropoff value for gapped alignment (in bits)
50 for blastn 25 for other programs

Accordingly, any nucleic acid sequence which encodes the amino acid sequence of an ErbB ligand can be used to produce recombinant molecules which express this ligand. In particular embodiments, the polynucleotide according to another aspect of the present invention encodes a polypeptide as set forth in SEQ ID NOS:73 to 84 and SEQ ID NOS:93, 95-104, 109-121, or a portion thereof, which modulates at least one biological, immunological or other functional characteristic or activity of a known ligand of at least one ErbB receptor.

The EGF-encoded variant domains disclosed herein comprise a consensus sequence that may be represented as follows: (X-8)-C-(X-7)-C-(X-2 to 3)-G-X-C-(X-10 to 13)-C-X, wherein X is any amino acid. This is the consensus pattern presented in FIG. 4. Shorter or longer amino-terminal sequences (X-8 hereinabove) can provide or define biological activity. Generally, synthetic peptides derived from the novel ligands may have extensions including an amino-terminal tail of amino acids.

It is to be understood that the present invention encompasses all fragments or variants including such amino terminal extensions, with the proviso that the C loop of the EGF domain is absent from these derivatives.

Methods for DNA sequencing are well known and generally available in the art, and may be used to practice any of the embodiments of the invention. The methods may employ such enzymes as the Klenow fragment of DNA polymerase I, Sequenase® (U.S. Biochemical Corp, Cleveland, Ohio), Taq polymerase (Perkin Elmer), thermostable T7 polymerase (Amersham, Chicago, Ill.), or combinations of polymerases and proofreading exonucleases such as those found in the ELONGASE Amplification System marketed by Gibco/BRL (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA Sequencers (Perkin Elmer).

It will be appreciated by those skilled in the art that as a result of the degeneracy of the genetic code, a multitude of nucleotide sequences encoding ErbB ligand isoforms, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring ErbB ligand isoforms, and all such variations are to be considered as being specifically disclosed.

Although nucleotide sequences which encode ErbB ligand isoforms and their variants are preferably capable of hybridizing to the nucleotide sequence of the naturally occurring ErbB ligand isoforms under appropriately selected conditions of stringency, it may be advantageous to produce nucleotide sequences encoding ErbB ligand isoforms or their derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding ErbB ligand isoforms and their derivatives without altering the encoded amino acid sequences include the production of RNA transcripts having more desirable properties, such as a greater half-life, than transcripts produced from the naturally occurring sequence.

The invention also encompasses production of DNA sequences, or fragments thereof, which encode ErbB ligand isoforms and their derivatives, entirely by synthetic chemistry. After production, the synthetic sequence may be inserted into any of the many available expression vectors and cell systems using reagents that are well known in the art. Moreover, synthetic chemistry may be used to introduce mutations into a sequence encoding ErbB ligand isoforms or any fragment thereof.

The present invention also includes polynucleotide sequences that are capable of hybridizing to the nucleotide sequences according to the present invention. According to one embodiment, the polynucleotide is preferably hybridizable with SEQ ID NOS: 73 to 84 and 93, 95-104, 109-121.

Hybridization for long nucleic acids (e.g., about 200 bp in length) is effected according to preferred embodiments of the present invention by stringent or moderate hybridization, wherein stringent hybridization is effected by a hybridization solution containing 10% dextran sulfate, 1 M NaCl, 1% SDS and 5×10⁶ rpm ³²P labeled probe, at 65° C., with a final wash solution of 0.2×SSC and 0.1% SDS and final wash at 65° C.; whereas moderate hybridization is effected by a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

According to preferred embodiments the polynucleotide according to this aspect of the present invention is as set forth in SEQ ID Nos:73 to 84 and 93, 95-104, 109-121, or a portion thereof, said portion preferably encodes a polypeptide comprising an amino acid stretch of at least 80%, preferably at least 85%, more preferably at least 90% or more, most preferably 95% or more identical to positions the polynucleotide sequence encoding the truncated ErbB receptor-modulating EGF domain devoid of the C-loop.

According to still another embodiment of the present invention there is provided an oligonucleotide of at least 17, at least 18, at least 19, at least 20, at least 22, at least 25, at least 30 or at least 40, bases specifically hybridizable with the isolated nucleic acid described herein.

Hybridization of shorter nucleic acids (below 200 bp in length, e.g., 1740 bp in length) is effected by stringent, moderate or mild hybridization, wherein stringent hybridization is effected by a hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 4.1% nonfat dried milk, hybridization temperature of 1-1.5° C. below the Tₘ, final wash solution of 3 M TMACI, 0.01 M sodium phosphate (pH 6.8), 1 m EDTA (pH 7.6), 0.5% SDS at 1-1.5° C. below the Tₘ. Moderate hybridization is effected by a hybridization solution of 6×SSC and 0.1% SDS or 3 M TMACI, 0.01 M sodium phosphate (pH 6.8), 1 mM EDTA (pH 7.6), 0.6% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; whereas mild hybridization is effected by a hybridization solution of 6×SSC and 1% SDS or 3M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 37° C., final wash solution of 6×SSC and final wash at 22° C.

According to an additional aspect of the present invention there is provided a pair of oligonucleotides each independently of at least 1740 bases specifically hybridizable with the isolated nucleic acid described herein in an opposite orientation so as to direct exponential amplification of a portion thereof, say of 50 to 2000 bp, in a nucleic acid amplification reaction, such as a polymerase chain reaction. The polymerase chain reaction and other nucleic acid amplification reactions are well known in the art and require no further description herein. The pair of oligonucleotides according to this aspect of the present invention are preferably selected to have comparable melting temperatures (Tₘ), e.g., melting temperatures which differ by less than that 7° C., preferably less than 5° C., more preferably less than 4° C., most preferably less than 3° C., ideally between 3° C. and 0° C. Consequently, according to yet an additional aspect of the present invention there is provided a nucleic acid amplification product obtained using the pair of primers described herein. Such a nucleic acid amplification product can be isolated by gel electrophoresis or by any other size-based separation technique. Alternatively, such a nucleic acid amplification product can be isolated by affinity separation, either stranded affinity or sequence affinity. In addition, once isolated, such a product can be further genetically manipulated by restriction, ligation and the like, to serve any one of a plurality of applications associated with regulation of ErbB activity as further detailed herein.

The nucleic acid sequences encoding ErbB ligand isoforms may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia, T. et al. (1988) Nucleic Acids Res. 16:8186). The primers may be designed using commercially available software such as OLIGO 4.06 Primer Analysis software (National Biosciences Inc., Plymouth, Minn.), or another appropriate program, to be 22-30 nucleotides in length, to have a GC content of preferably but not exclusively between 40% to 60%, and to anneal to the target sequence at temperatures about 68° C. to 72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom, M. et al. (1991) PCR Methods Applic. 1:111-119). In this method, multiple restriction enzyme digestions and ligations may also be used to place an engineered double-stranded sequence into an unknown fragment of the DNA molecule before performing PCR.

Another method which may be used to retrieve unknown sequences is that of Parker, J. D. et al. (1991; Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences which contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

After defining novel segments of genomic DNA methods to generate novel transcripts, e.g., primer extension, plating and isolation of cDNA cosmid/plasmid clones, RT-PCR using contrived primers guessed from exon prediction programs which read through genomic DNA sequences may be applied as is well known in the art.

Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled devise camera. Output/light intensity may be converted to electrical signal using appropriate software (e.g. Genotyper™ and Sequence Navigator™, Perkin Elmer) and the entire process from loading of samples to computer analysis and electronic data display may be computer controlled. Capillary electrophoresis is especially preferable for the sequencing of small pieces of DNA which might be present in limited amounts in a particular sample.

Thus, this aspect of the present invention encompasses (i) polynucleotides as set forth in SEQ ID NOs: DNA sequence IDs claimed (exclusive of the known gamma isoform):128 to 139, 148, 150-159 and 164-176; (ii) fragments thereof; (iii) sequences hybridizable therewith; (iv) sequences homologous thereto; (v) sequences encoding similar polypeptides with different codon usage; (vi) altered sequences characterized by mutations, such as deletion, insertion or substitution of one or more nucleotides, either naturally occurring or man induced, either randomly or in a targeted fashion.

### Constructs Comprising the Novel Variants

According to another aspect of the present invention there is provided a nucleic acid construct comprising the isolated nucleic acid described herein.

According to a preferred embodiment the nucleic acid construct according to this aspect of the present invention further comprising a promoter for regulating the expression of the isolated nucleic acid in a sense or antisense orientation. Such promoters are known to be cis-acting sequence elements required for transcription as they serve to bind DNA dependent RNA polymerase which transcribes sequences present downstream thereof. Such down stream sequences can be in either one of two possible orientations to result in the transcription of sense RNA which is translatable by the ribosome machinery or antisense RNA which typically does not contain translatable sequences, yet can duplex or triplex with endogenous sequences, either mRNA or chromosomal DNA and hamper gene expression, all as is further detailed hereinunder.

While the isolated nucleic acid described herein is an essential element of the invention, it is modular and can be used in different contexts. The promoter of choice that is used in conjunction with this invention is of secondary importance, and will comprise any suitable promoter sequence. It will be appreciated by one skilled in the art, however, that it is necessary to make sure that the transcription start site(s) will be located upstream of an open reading frame. In a preferred embodiment of the present invention, the promoter that is selected comprises an element that is active in the particular host cells of interest. These elements may be selected from transcriptional regulators that activate the transcription of genes essential for the survival of these cells in conditions of stress or starvation, including the heat shock proteins.

### Vectors and Host Cells

In order to express a biologically active ErbB ligand isoform, the nucleotide sequences encoding ErbB ligand isoforms or functional equivalents according to the present invention may be inserted into appropriate expression vector, i.e., a vector which contains the necessary elements for the transcription and translation of the inserted coding sequence.

Vectors can be introduced into cells or tissues by any one of a variety of known methods within the art, including in vitro recombinant DNA techniques, synthetic techniques, and in vivo genetic recombination. Such methods are generally described in Sambrook et al., Molecular Cloning: A Laboratory Manual, Cold Springs Harbor Laboratory, New York 1989, 1992; in Ausubel et al., Current Protocols in Molecular Biology, John Wiley and Sons, Baltimore, Md. 1989; Chang et al., Somatic Gene Therapy, CRC Press, Ann Arbor, Mich. 1995; Vega et al., Gene Targeting, CRC Press, Ann Arbor Mich. 1995; Vectors: A Survey of Molecular Cloning Vectors and Their Uses, Butterworths, Boston Mass. 1988; and Gilboa et al. (1986) Biotechniques 4 (6): 504-512, and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

A variety of expression vector/host systems may be utilized to contain and express sequences encoding ErbB ligand isoforms. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. The invention is not limited by the host cell employed. The expression of the construct according to the present invention within the host cell may be transient or it may be stably integrated in the genome thereof.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The “control elements” or “regulatory sequences” are those non-translated regions of the vector-enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the Bluescript® phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding variant ErbB-ligand, vectors based on SV40 or EBV may be used with an appropriate selectable marker.

In bacterial systems, a number of expression vectors may be selected depending upon the use intended for variant ErbB-ligand expression. For example, when large quantities of variant ErbB-ligand are needed for the induction of antibodies, vectors which direct high level expression of fusion proteins that are readily purified may be used. Such vectors include, but are not limited to, the multifunctional E. coli cloning and expression vectors such as Bluescript® (Stratagene), in which the sequence encoding variant ErbB-ligand may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding variant ErbB-ligand may be driven by any of a number of promoters. For example, viral promoters such as the 35S and 19S promoters of CaMV may be used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196.

An insect system may also be used to express variant ErbB-ligand. For example, in one such system, Autographa californica nuclear polyhedrosis virus (AcNPV) is used as a vector to express foreign genes in Spodoptera frugiperda cells or in Trichoplusia larvae. The sequences encoding variant ErbB-ligand may be cloned into a non-essential region of the virus, such as the polyhedrin gene, and placed under control of the polyhedrin promoter. Successful insertion of variant ErbB-ligand will render the polyhedrin gene inactive and produce recombinant virus lacking coat protein. The recombinant viruses may then be used to infect, for example, S. frugiperda cells or Trichoplusia larvae in which variant ErbB-ligand may be expressed (Engelhard, E. K. et al. (1994) Proc. Nat. Acad. Sci. 91:3224-3227).

In mammalian host cells, a number of viral-based expression systems may be utilized. In cases where an adenovirus is used as an expression vector, sequences encoding variant ErbB-ligand may be ligated into an adenovirus transcription/translation complex consisting of the late promoter and tripartite leader sequence. Insertion in a non-essential E1 or E3 region of the viral genome may be used to obtain a viable virus which is capable of expressing variant ErbB-ligand in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of 6 to 10M are constructed and delivered via conventional delivery methods (liposomes, polycationic amino polymers, or vesicles) for therapeutic purposes.

Specific initiation signals may also be used to achieve more efficient translation of sequences encoding variant ErbB-ligand. Such signals include the ATG initiation codon and adjacent sequences. In cases where sequences encoding variant ErbB-ligand, its initiation codon, and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

### Polypeptide Purification

Host cells transformed with nucleotide sequences encoding ErbB ligand isoforms may be cultured under conditions suitable for the expression and recovery of the protein from cell culture. The protein produced by a transformed cell may be secreted or contained intracellularly depending on the sequence and/or the vector used. The polynucleotide encoding for ErbB ligand isoforms may include a signal peptide which direct secretion of ErbB ligand isoforms through a prokaryotic or eukaryotic cell membrane. Other constructions may be used to join sequences encoding ErbB ligand isoforms to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.). The inclusion of cleavable linker sequences, such as those specific for Factor XA or enterokinase (Invitrogen, San Diego, Calif.), between the purification domain and the ErbB ligand isoforms encoding sequence may be used to facilitate purification. One such expression vector provides for expression of a fusion protein containing ErbB ligand isoforms and a nucleic acid encoding 6 histidine residues preceding a thioredoxin or an enterokinase cleavage site. The histidine residues facilitate purification on immobilized metal ion affinity chromatography. (IMIAC) (See, e.g., Porath, J. et al. (1992) Prot. Exp. Purif. 3:263-281.) The enterokinase cleavage site provides a means for purifying ErbB ligand isoforms from the fusion protein. (See, e.g., Kroll, D. J. et al. (1993) DNA Cell Biol. 12:441-453.)

Fragments of ErbB ligand isoforms may be produced not only by recombinant production, but also by direct peptide synthesis using solid-phase techniques. (See, e.g., Creighton, T. E. (1984) Protein: Structures and Molecular Properties, pp. 55-60, W. H. Freeman and Co. New York, N.Y.) Protein synthesis may be performed by manual techniques or by automation. Automated synthesis may be achieved, for example, using the Applied Biosystems 431A peptide synthesizer (Perkin Elmer). Various fragments of ErbB ligand isoforms may be synthesized separately and then combined to produce the full length molecule.

### Transgenic Animals or Cell Lines

The present invention has the potential to provide transgenic gene and polymorphic gene animal and cellular (cell lines) models as well as for knock-out and knock-in models. These models may be constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson (1991) Methods in Enzymology, 194:251-270; Capecchi (1989) Science 244:1288-1292; Davies et al. (1992) Nucleic Acids Research, (11) 2693-2698; Dickinson et al. (1993) Human Molecular Genetics, 2(8): 1299-1302; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al. (1991) Genomics, 9:7414 750 1991; Jakobovits et al. (1993) Nature, 362:255-261; Lamb et al. (1993) Nature Genetics, 5: 22-29; Pearson and Choi, (1993) Proc. Natl. Acad. Sci. USA 90:10578-82; Rothstein, (1991) Methods in Enzymology, 194:281-301; Schedl et al. (1993) Nature, 362: 258-261; Strauss et al. (1993) Science, 259; 1904-1907. Further, patent applications WO 94/23049, WO 93/14200, WO 94/06408, WO 94/28123 also provide information.

All such transgenic gene and polymorphic gene animal and cellular (cell lines) models and knockout or knock-in models derived from claimed embodiments of the present invention, constitute preferred embodiments of the present invention.

### Gene Therapy

Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a ligand, hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (i) ex vivo and (ii) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject. Rather, the genetic material to be transferred is introduced into the cells of the recipient organism in situ, that is within the recipient. In an alternative embodiment, if the host gene is defective, the gene is repaired in situ (Culver, 1998. (Abstract) Antisense DNA & RNA based therapeutics, February 1998, Coronada, Calif.). These genetically altered cells have been shown to express the transfected genetic material in situ. The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequences which work contiguously with the coding sequence (in cis) to change the basal transcription level dictated by the promoter. The expression vehicle can also include a selection gene as described hereinbelow.

### Vectors Useful in Gene Therapy

As described herein above, vectors can be introduced into host cells or tissues by any one of a variety of known methods within the art.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Features that limit expression to particular cell type can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The natural specificity of viral vectors is utilized to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles, which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, they do not have to be administered locally at the diseased site. However, when local administration can provide a quicker and more effective treatment, administration can also be performed by, for example, intravenous or subcutaneous injection into the subject. Injection of the viral vectors into a spinal fluid can also be used as a mode of administration. Following injection, the viral vectors will circulate until they recognize cells with appropriate target specificity for infection.

Thus, according to an alternative embodiment, the nucleic acid construct according to the present invention further includes a positive and a negative selection markers and may therefore be employed for selecting for homologous recombination events, including, but not limited to, homologous recombination employed in knock-in and knockout procedures. One ordinarily skilled in the art can readily design a knockout or knock-in constructs including both positive and negative selection genes for efficiently selecting transfected embryonic stem cells that underwent a homologous recombination event with the construct.

Such cells can be introduced into developing embryos to generate chimeras, the offspring thereof can be tested for carrying the knockout or knock-in constructs.

Knockout and/or knock-in constructs according to the present invention can be used to further investigate the functionality of ErbB ligand isoforms. Such, constructs can also be used in somatic and/or germ cells gene therapy to increase/decrease the activity of ErbB signaling, thus regulating ErbB related responses. Further detail relating to the construction and use of knockout and knock-in constructs can be found in Fukushige, S. and Ikeda, J. E. (1996) DNA Res 3:73-50; Bedell, M. A. et al. (1997) Genes and Development 11:1-11; Bermingham, J. J. et al. (1996) Genes Dev 10:1751-1762, which are incorporated herein by reference as if set forth herein.

### Antisense

According to still an additional aspect of the present invention there is provided an antisense oligonucleotide comprising a polynucleotide or a polynucleotide analog of at least 10 bases, preferably between 10 and 15, more preferably between 5 and 20 bases, most preferably, at least 17-40 bases being hybridizable in vivo, under physiological conditions, with a portion of a polynucleotide strand encoding a polypeptide at least 80%, preferably at least 85%, more preferably at least 90% or more, most preferably at least 95% or more homologous (similar+identical acids) to the sequence of the ErbB receptor-modulating EGF ligand devoid of the C-loop disclosed by the present invention. Such antisense oligonucleotides can be used to downregulate expression as further detailed hereinunder. Such an antisense oligonucleotide is readily synthesizable using solid phase oligonucleotide synthesis. The ability of chemically synthesizing oligonucleotides and analogs thereof having a selected predetermined sequence offers means for down-modulating gene expression. Three types of gene expression modulation strategies may be considered. At the transcription level, antisense or sense oligonucleotides or analogs that bind to the genomic DNA by strand displacement or the formation of a triple helix, may prevent transcription. At the transcript level, antisense oligonucleotides or analogs that bind target mRNA molecules lead to the enzymatic cleavage of the hybrid by intracellular RNase H. In this case, by hybridizing to the targeted mRNA, the oligonucleotides or oligonucleotide analogs provide a duplex hybrid recognized and destroyed by the RNase H enzyme. Alternatively, such hybrid formation may lead to interference with correct splicing. As a result, in both cases, the number of the target mRNA intact transcripts ready for translation is reduced or eliminated. At the translation level, antisense oligonucleotides or analogs that bind target mRNA molecules prevent, by steric hindrance binding of essential translation factors (ribosomes), to the target mRNA a phenomenon known in the art as hybridization arrest, disabling the translation of such mRNAs.

Thus, antisense sequences, which as described hereinabove may arrest the expression of any endogenous and/or exogenous gene depending on their specific sequence, attracted much attention by scientists and pharmacologists who were devoted at developing the antisense approach into a new pharmacological tool. For example, several antisense oligonucleotides have been shown to arrest hematopoietic cell proliferation (Szczylik et al., 1991), growth (Calabretta et al.; 1941), entry into the S phase of the cell cycle (Heikhila et al., 1987), reduced survival (Reed et al., 1990) and prevent receptor mediated responses (Burch and Mahan, 1991). For efficient in vivo inhibition of gene expression using antisense oligonucleotides or analogs, the oligonucleotides or analogs must fulfill the following requirements (i) sufficient specificity in binding to the target sequence; (ii) solubility in water; (iii) stability against intra- and extracellular nucleases; (iv) capability of penetration through the cell membrane; and (v) when used to treat an organism, low toxicity. Unmodified oligonucleotides are typically impractical for use as antisense sequences since they have short in vivo half-lives, during which they are degraded rapidly by nucleases. Furthermore, they are difficult to prepare in more than milligram quantities. In addition, Such oligonucleotides are poor cell membrane penetrators. Thus it is apparent that in order to meet all the above listed requirements, oligonucleotide analogs need to be devised in a suitable manner. Therefore, an extensive search for modified oligonucleotides has been initiated. For example, problems arising in connection with double-stranded DNA (dsDNA) recognition through triple helix formation have been diminished by a clever “switch back” chemical linking, whereby a sequence of polypurine on one strand is recognized, and by “switching back”, a homopurine sequence on the other strand can be recognized. Also, good helix formation has been obtained by using artificial bases. thereby improving binding conditions with regard m ionic strength and pH.

Oligonucleotide Analogs

In addition, in order to improve half-life as well as membrane penetration, a large number of variations in polynucleotide backbones have been done. Oligonucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate brides, carboxymethyl ester bridges, carbonate bridges, carboxymethyl ester bridges; acetamide bridges, carbonate bridges, thioether bridges, sulfoxy bridges, sulfono bridges, various “plastic” DNAs, α-anomeric bridges and borane derivatives. International patent application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking or ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO₂—). International patent application WO 92/20702 describe an acyclic oligonucleotide which includes a peptide backbone on which any selected chemical nucleobases or analogs are stringed and serve an coding characters as they do in natural DNA or RNA. These new compounds, known as peptide nucleic acids (PNAs), are not only more stable in cells than their natural counterparts, but also bind natural DNA and RNA, 50 to 100 times more tightly than the natural nucleic acids cling to each other. PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal region.

Thus, in one preferred aspect antisense technology requires pairing of messenger RNA wish an oligonucleotide to form a double helix that inhibits translation. The concept of antisense-mediated gone therapy was already introduced in 1978 for cancer therapy. This approach was based on certain genes that are crucial in cell division and growth of cancer cell. Synthetic fragments of genetic substance DNA can achieve this goal. Such molecules bind to the targeted gene molecules in RNA of tumor cells, thereby inhibiting the translation of the gates and resulting in dysfunctional growth of these cells. Other mechanisms has also been proposed. These strategies have been used, with some success is treatment of cancers, as well or other illnesses, including viral and other infectious diseases. Antisense oligonucleotides are typically synthesized in lengths of 13-30 nucleotides. The life span of oligonucleotide molecules in blood is rather shots.

Thus, they have to be chemically modified to prevent destruction by ubiquitous nucleases present in the body. Phosphorothioates are very widely used modification in antisense oligonucleotide ongoing clinical trials. A new generation of antisense molecules consist of hybrid antisense oligonucleotide with a central portion of synthetic DNA while four bases on each end have been modified with 2′O-methyl ribose to resemble RNA. In preclinical studies in laboratory animals, such compounds have demonstrated greater stability to metabolism in body tissues and an improved safety profile when compared with the first-generation unmodified phosphorothioate (Hybridon Inc. news). Dozens of other nucleotide analogs have also been tested in antisense technology.

RNA oligonucleotides tray also be used for antisense inhibition as they form a stable RNA-RNA duplex with the target, suggesting efficient inhibition However, due to their low stability RNA oligonucleotides are typically expressed inside the cells using vectors designed for this purpose. This approach is favored when attempting to target a mRNA that encodes an abundant and long-lived protein.

Recent scientific publications have validated the efficacy of antisense compounds in animal models of hepatitis, cancers, coronary artery restenosis and other diseases. The first antisense drug was recently approved by the FDA. This drug Fomivirsen, developed by Isis, is indicated for local treatment of cytomegalovirus in patients with AIDS who are intolerant of or have a contraindication to other treatments for CMV retinitis or who were insufficiently responsive to previous treatments for CMV retinitis (Pharmacotherapy News Network).

Several antisense compounds are now in clinical trials in the United States. These include locally administered antivirals, systemic cancer therapeutics. Antisense therapeutics has the potential to treat many life threatening diseases with a number of advantages over traditional drugs. Traditional drugs intervene after a disease-causing protein is formed. Antisense therapeutics, however, block mRNA transcription/translation and intervene before a protein is formed, and since antisense therapeutics target only one specific mRNA, they should be more effective with fewer side effects than current protein-inhibiting therapy.

A second option for disrupting gene expression at the level of transcription uses synthetic oligonucleotides capable of hybridizing with double stranded DNA. A triple helix is formed. Such oligonucleotides may prevent binding of transcription factors to the gene's promoter and therefore inhibit transcription. Alternatively they may prevent duplex unwinding and, therefore, transcription of genes within the triple helical structure.

Thus, according to a further aspect of the present invention there is provided a pharmaceutical composition comprising the antisense oligonucleotide described herein and a pharmaceutically acceptable carries. The pharmaceutically acceptable carrier can be, for example, a liposome loaded with the antisense oligonucleotide. Formulations for topical administration may include, but are not limited to, lotions, ointments, gels, creams, suppositories, drops, liquids, sprays and powders. Conventional pharmaceutical carriers, aqueous, powder or oily bases, thickeners and the like may be necessary or desirable. Compositions for oral administration include powders or granules, suspensions or solutions in water or non-aqueous media, sachets, capsules or tablets. Thickeners, diluents, flavorings, dispersing aids, emulsifiers or binders may be desirable. Formulations for parenteral administration may include but are not limited to, sterile aqueous solutions which tray also contain buffers, diluents and other suitable additives.

According to still a further aspect of the present invention there is provided a ribozyme comprising the antisense oligonucleotide described herein and a ribozyme sequence fused thereto. Such a ribozyme is readily synthesizable using solid phase oligonucleotide synthesis.

Ribozymes are being increasingly used for the sequence-specific inhibition of gene expression by the cleavage of mRNAs encoding proteins of interest. The possibility of designing ribozymes to cleave any specific target RNA has rendered them valuable toots in both basic research and therapeutic applications. In the therapeutics area, ribozymes have been exploited to target viral RNAs in infectious diseases, dominant oncogenes in cancers and specific somatic mutations in genetic disorders. Most notably, several ribozyme gene therapy protocols for HIV patients are already in Phase 1 trials. More recently, ribozymes have been used for transgenic animal research, gene target validation and pathway elucidation Several ribozymes are in various stages of clinical trials. ANGIOZYME was the first chemically synthesized ribozyme to be studied in human clinical orals. ANGIOZYME specifically inhibits formation of VEGF-r (Vascular Endothelial Growth Factor receptor), a key component in the angiogenesis pathway, Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated-WEB home page). According to yet a further aspect of the present invention there is provided a recombinant or synthetic (i.e., prepared using solid phase peptide synthesis) protein comprising a polypeptide capable of modulating an ErbB receptor and which is at least 80%, preferably at least 85%, more preferably at least 90% or more, most preferably at least 95% or more or 100% identical or homologous (identical+similar) to a novel splice variant comprising the receptor modulating EGF domain of an ErbB ligand with the proviso that said ligand is devoid of the C-loop of the receptor modulating EGF domain. Most preferably the polypeptide includes at least a portion of the ErbB ligand splice variants of the present invention that may include amino acids spanning cyteines 1 to 4 but are absent cysteines 5 and 6 of the receptor modulating EGF domain. Additionally or alternatively, the polypeptide according to this aspect of the present invention is preferably encoded by a polynucleotide hybridizable with SEQ ID NOs: 128 to 139. 148, 150-159 and 164-176, or a portion thereof under any of the stringent or moderate hybridization conditions described above for long nucleic acids. Still additionally or alternatively, the polypeptide according to this aspect of the present invention is preferably encoded by a polynucleotide at least 80%, at least 85%, at least 90%, at least 95%, or 100%, identical with the sequences disclosed herein that encode the splice variants lacking the C-loop of the receptor modulating EGF domain.

Thus, this aspect of the present invention encompasses (i) polypeptides as set forth in SEQ ID NOs: 73 to 84 and 93, 95-104, 109-121; (ii) fragments thereof; (iii) polypeptides homologous thereto; and (iv) altered polypeptide characterized by mutations, such as deletion, insertion or substitution of one or more amino acids, either naturally occurring or man induced, either random or in a targeted fashion, either natural, non-natural or modified at or after synthesis, with the proviso that the C-loop is absent form the receptor modulating domain.

According to still a further aspect of the present invention there is provided a pharmaceutical composition comprising, as an active ingredient the recombinant protein described herein and a pharmaceutical acceptable carrier which is further described above.

Peptides

As used herein in the specification and in the claims section below the phrase “derived from a polypeptide” refers to peptides derived from the specified protein or proteins and further to homologous peptides derived from equivalent regions of proteins homologous to the specified proteins of the same or other species. The term further relates to permissible amino acid alterations and peptidomimetics designed based on the amino acid sequence of the specified proteins or their homologous proteins.

As used herein in the specification and in the claims section below the term “amino acid” is understood to include the 20 naturally occurring amino acids; those amino acids often modified post-translationally in vivo, including for example hydroxyproline, phosphoserine and phosphothreonine; and other unusual amino acids including, but not limited to, 2-aminoadipic acid: hydroxylysine isodesmosine, nor-valine, nor-leucine and ornithine. Furthermore, the term “amino acid” includes both D- and L-amino acids, Further elaboration of the possible amino acids usable according to the present invention and examples of non-natural amino acids are given hereinunder. Hydrophilic aliphatic natural amino acids can be substituted by synthetic amino acids, preferably Nleu, Nval and/or α-aminobutyric acid or by aliphatic amino acids of the general formula —HN(CH₂)ₙCOOH, wherein n=3-5, as well as by branched derivatives thereof, wherein an alkyl group, for example, methyl, ethyl or propyl, is located at any one or more of the n carbons.

Each one, or more, of the amino acids can include a D-isomer thereof. Positively charged aliphatic carboxylic acids, such as, but not limited to, H₂N(CH₂), COOH, wherein n=24 and H₂N—C(NH)—NH(CH₂)ₙCOOH, wherein n=2-3, as well as by hydroxy Lysine, N-methyl Lysine or ornithine (Orn) can also be employed. Additionally, enlarged aromatic residues, such as, but not limited to, H₂N—(C₆H₆)—CH₂—COOH, p-aminophenyl alanine, H₂N—F(NH)—NH—(C₆H₆)—CH₂—COOH, p-guanidinophenyl alanine or pyridinoalanine (Pal) can also be employed. Side chains of amino acid derivatives (if these are Ser, Tyr, Lys, Cys or Orn) can be protected-attached to alkyl, aryl, alkyloyl or aryloyl moieties. Cyclic derivatives of amino acids can also be used. Cyclization can be obtained through amide bond formation, e.g., by incorporating Glu, Asp, Lys, Orn, di-amino butyric (Dab) acid, di-aminopropionic (Dap) acid at various positions is the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COON)—C(R)H—NH₂, wherein n=1-4, and further wherein R is any natural or non-natural side chain of an amino acid. Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap, Peptide bonds (—CO—NH—) within the peptide may be substituted by N-methylated bonds (—N(CH₃)—CO—), ester bonds (—C(R)H—CO—O—C(R)—N—), ketomethylene bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (2-3) at the same time. Natural aromatic amino acids, Trp, Tyr and Phe, may be substituted far synthetic port-natural acid such as TIC, naphthylelanine (Nol), ring-methylated derivatives of Phe, halogenated derivatives of Phe or o-methyl Tyr.

### Display Libraries

According to still another aspect of the present invention there is provided a display library comprising a plurality of display vehicles (such as phages, viruses or bacteria) each displaying at least 5-10 or 15-20 consecutive amino acids derived from a polypeptide at least 80%, at least 85%, at least 90%, at least 95%, or 100% identical or homologous (identical+similar) to SEQ ID Nos:73 to 84 and 93, 95-104, 109-121.

According to a preferred embodiment of this aspect of the present invention substantially every 5-10 or 15-20 consecutive amino acids derived from the polypeptide at least 80%, at least 85%, at least 90%, at least 95%, or 100% identical or homologous (identical+similar) to SEQ ID NOs:73 to 84 and SEQ ID NOS:93, 95-104, 109-121 are displayed by at least one at the plurality of display vehicles, so as to provide a highly representative library. Preferably, the consecutive amino acids or amino acid analogs of the peptide or peptide analog according to this aspect of the present invention are derived from SEQ ID NOs.:73 to 84 and 93, 95-104, 109-121, with the proviso that these peptides are devoid of the C-loop of the EGF domain. Methods of constructing display libraries are well known in the art, such methods are described, for example, in Young A C, et al., J Mol Biol 1997; 274(4):622-34; Giebel L B et al. Biochemistry 1995; 34 (47):15430-5; Davies E L et al., J Immunol Methods 1995; 186(1):125-35; Jones C et al. J Chromatogr A 1995; 707(1):3-22; Deng S J et al. Proc Natl Acad Sci USA 1995; 92(11):4992-6; and Deng S J et al. J Biol Chem 1994; 269(13):9533-8, which are incorporated herein by reference. Display libraries according to this aspect of the present invention can be used to identify and isolate polypeptides and variants which are capable of up- or down-regulating ErbB activity.

### Antibodies

According to still another aspect of the present invention there is provided an antibody comprising at least the antigen binding portion of an immunoglobulin specifically recognizing and binding a polypeptide at least 80%, at least 85%, at least 90%, at least 95%, or 100% identical or homologous (identical+similar) to SEQ ID NOs: 73 to 84 and 93, 95-104, 109-121 with the proviso that these antibodies do not bind significantly to the C-loop of an intact EGF domain.

The present invention can utilize serum immunoglobulins, polyclonal antibodies or fragments thereof, (i.e., immunoreactive derivative of an antibody), or monoclonal antibodies or fragments thereof. Monoclonal antibodies of purified fragments of the monoclonal antibodies having at least a portion of an antigen bidding region, including such as Fv, F(abl)₂, Fab fragments (Harlow and Lane, 1988 Antibody, Cold Spring Harbor); single chain antibodies (U.S. Pat. No. 4,946,778), chimeric or humanized antibodies and complementarily determining regions (CDR) may be prepared by conventional procedures. Purification of these serum immunoglobulins antibodies or fragments can be accomplished by a variety of methods known to those of skill including, precipitation by ammonium sulfate or sodium sulfate followed by dialysis against saline, ion exchange chromatography, affinity or immunoaffinity chromatography as well as gel filtration, zone electrophoresis, etc. (see Goding in, Monoclonal Antibodies: Principles and Practice, 2nd ed., pp. 104-126, 1986, Orlando, Fla., Academic Press). Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains. Additional classes includes IgD, IgE, IgA, IgM and related proteins.

### Monoclonal Antibodies

Methods for the generation and selection of monoclonal antibodies are well known in the art, as summarized for example in reviews such as Tramontano and Schloeder, Methods in Enzymology 178, 551-568, 1989. A recombinant or synthetic ErbB ligand or a portion thereof of the present invention may be used to generate antibodies in vitro. More preferably, the recombinant or synthetic ErbB ligand of the present invention is used to elicit antibodies in vivo. In general, a suitable host animal is immunized with the recombinant or synthetic ErbB ligand of the present invention or a portion thereof including at least one continuous or discontinuous epitope. Advantageously, the animal host used is a mouse of an inbred strain. Animals are typically immunized with a mixture comprising a solution of the recombinant or synthetic ErbB ligand of the present invention or portion thereof in a physiologically acceptable vehicle, and any suitable adjuvant, which achieves as enhanced immune response to the immunogen. By way of example, the primary immunization conveniently may be accomplished with a mixture of a solution of the recombinant or synthetic ErbB ligand of the present invention or a portion thereof and Freund's complete adjuvant, said mixture being prepared in the form of a water-in-oil emulsion. Typically the immunization may be administered to the animals intramuscularly, intradermally, subcutaneously, intraperitoneally, into the footpads, or by any appropriate route of administration. The immunization schedule of the immunogen may be adapted as required, but customarily involves several subsequent or secondary immunizations using a milder adjuvant such as Freund's incomplete adjuvant.

Antibody titers and specificity of binding can be determined during the immunization schedule by any convenient method including by way of example radioimmunoassay, or enzyme linked immunosorbant assay, which is known as the ELISA assay. When suitable antibody titers are achieved, antibody producing lymphocytes from the immunized animals are obtained, and these are cultured, selected and closed, as is known in the art. Typically, lymphocytes may be obtained in large numbers from the spleens of immunized animals, but they may also be retrieved from the circulation, the lymph nodes or other lymphoid organs. Lymphocyte are then fused with any suitable myeloma cell line, to yield hybridomas, as is well known in the art. Alternatively, lymphocytes may also be stimulated to grow in culture; and may be immortalized by methods known in the art including the exposure of these lymphocytes to a virus; a chemical or a nucleic acid such as an oncogene, according to established protocols. After fusion, the hybridomas are cultured under suitable culture conditions, for example in multiwell plates, and the culture supernatants are screened to identify cultures containing antibodies that recognize the hapten of choice. Hybridomas that secrete antibodies that recognize the recombinant or synthetic NRG-4 of the present invention are cloned by limiting dilution and expanded, under appropriate culture conditions. Monoclonal antibodies are purified and characterized in terms of immunoglobulin type and binding affinity.

### Pharmaceutical Compositions for Regulation of ErbB Receptor Activity

According to yet another aspect of the present invention there is provided a pharmaceutical composition comprising, as an active ingredient, an agent for regulating an ErbB receptor mediated activity in vivo or in vitro. The following embodiments of the present invention are directed at intervention with ErbB ligand activity and therefore with ErbB receptor signaling.

According to yet another aspect of the present invention there is provided a method of regulating an endogenous protein affecting ErbB receptor activity in vivo or in vitro. The method according to this aspect of the present invention is effected by administering an agent for regulating the endogenous protein activity in vivo, the endogenous protein being at least 80%, at least 85%, at least 90%, at least 95%, or 100% identical or homologous (identical+similar) to SEQ ID NOs: 73 to 84 and 93, 95-104, 109-121, with the proviso that it is devoid of the C-loop of the intact EGF domain.

An agent which can be used according to the present invention to upregulate the activity of the endogenous protein can include, for example, an expressible sense polynucleotide at least 80%, at least 85%, at least 90%, at least 95%, or 100% identical with SEQ ID NOs:128 to 139, 148, 150-159, 164-176, with the proviso that it does not encode the C-loop of the intact EGF domain.

An agent which can be used according to the present invention to down-regulate the activity of the endogenous protein can include, for example, an expressible antisense polynucleotide at least 80%, at least 85%, at least 90%, at least 95%, or 100%, identical with a portion of SEQ ID Nos:128 to 139, 148, 150-159, 164-176, with the proviso that it does not encode the C-loop of the intact EGF domain. Alternatively, an agent which can be used according to the present invention to downregulate the activity of the endogenous protein can include, for example, an antisense oligonucleotide or ribozyme which includes a polynucleotide or a polynucleotide analog of at least 10 bases, preferably between 10 and 15, more preferably between 15 and 20 bases, most preferably, at least 17-40 bases which is hybridizable in vivo, under physiological conditions, with a portion of a polynucleotide strand encoding a polypeptide at least 80%, at least 85%, at least 90%, at least 95%, or 100% identical or homologous (identical+similar) to SEQ ID NOs:128 to 139 and, 148, 150-159, 164-176, Still alternatively, an agent which can be used according to the present invention to downregulate the activity of the endogenous protein can include, for example, an peptide or a peptide analog representing a stretch of at least 6-10, 10-15, or 15-20 consecutive amino acids or analogs thereof derived from a polypeptide at least 80%, at least 85%, at least 90%, at least 95%, or 100% identical or homologous (identical+similar) to SEQ ID NOs: 73 to 84 and SEQ ID NOS:93, 95-104, 109-121.

Peptides or peptide analogs containing the interacting EGF-like domain according to the present invention will compete by protein interactions to form protein complexes with ErbB receptor, inhibiting or accelerating the pathways in which ErbB ligands are involved.

The following biochemical and molecular systems are known for the characterization and identification of protein-protein interaction and peptides as substrates, through peptide analysis, which systems can be used to identify inhibitory peptide sequences. One such system employs introduction of a genetic material encoding a functional protein or a mutated form of the protein, including amino acid deletions and substitutions, into cells. This system, can be used to identify functional domains of the protein by the analysis of its activity and the activity of its derived mutants in the cells. Another such system employs the introduction of small encoding fragments of a gene into cells, e.g., by means of a display library or a directional randomly primed cDNA library comprising fragments of the gene, and analyzing the activity of the endogenous protein in their presence (see, for example, Gudkov et al. 1993, Proc. Natl. Acad. Sci. USA 90:3231-3236; Gudkov and Robinson (1997) Methods Mol Biol 69; 221-240; and Pestov et al. 1999, Bio Techniques 26:102-106). Yet an additional system is realized by screening expression libraries with peptide domains, as exemplified, for example, by Yamabhai et al. 1998, J Biol Chem 273: 31401-31407). In yet another such system overlapping synthetic peptides derived from specific gene products are used to study and affect in vivo and in vitro protein-protein interactions. For example, synthetic overlapping peptides derived from the HIV-1 gene (20-30 amino acids) were assayed for different viral activities (Baraz et al. 1998, FEBS Letters 441:419-426) and were found to inhibit purified viral protease activity; bind to the viral protease; inhibit the Gag-Pol polyprotein cleavage; and inhibit mature virus production in human cells.

The following examples are provided solely for purposes of illustration of the principles of the invention and are not intended to limit the scope of the invention in any manner.

### EXAMPLES

### Synthetic Peptides Comprising the Novel Variants

Peptides were synthesized on an Applied Biosystems (ABI) 430A peptide synthesizer using standard tert-butyloxycarbonyl (t-Boc) chemistry protocols as provided (version 1.40; N-methylpyrrolidonelhydroxybenzotriazole). Acetic anhydride capping was employed after each activated ester coupling. The peptides were assembled on phenylacetamidomethyl polystyrene resin using standard side chain protection except for the use of t-Boc Glu(O-cyclohexyl) and t-Boc Asp(O-cyclohexyl). The peptides were deprotected using the “Low-High” hydrofluoric acid (HF) method of Tam et al. (J. Am. Chem. Soc. 105:6442 (1983)). In each case crude HF product was purified by reverse phase HPLC (C-18 Vydac, 22×250 mm), diluted without drying into folding buffer (1 M urea, 100 mM Tris, pH 8.0, 1.5 mM oxidized glutathione, 0.75 mM reduced glutathione, 10 mM Met), and stirred for 48 h at 4° C. Folded, fully oxidized peptides were purified from the folding mixture by reverse phase HPLC and characterized by electrospray mass spectroscopy; quantities were determined by amino acid analysis.

### Bioinformatics

EST, genomic and non redundant databases were searched for homology particularly to the EGF-like domains of various ErbB ligands by BLAST and Smith-Waterman based searches (Altschul et al., 1997; Samuel and Altschul, 1990; Smith and Waterman, 1981). BLASTN, BLASTP and TBLASTN—based searches were performed using the National Center for Biological Information (NCBI) node, utilizing both the search engines and databases offered at this site. Multiple sequence alignments were performed using ClustalX (Version 1.81 for Windows); (Chema et. al. 2003). Smith-Waterman based searches were performed using a software package and Compugen Bioccelerator maintained at the European Molecular Biology Laboratory (EMBL-interface). Profile-based searches were also performed using this Bioccelerator; Sequence profiles were generated from ClustalX multiple sequence alignments of proteins using the software PROFILEWEIGHT, which is provided as a software component of the EMBL-interface Compugen Bioccelerator. Profile searches were then performed against DNA databases, using the program TPROFILESEARCH (Compugen Bioccelerator at EMBL; program version 1.9). The databases scanned for the Bioccelerator searches were in this case maintained at the EMBL site.

Sequences of defined names or accession numbers were retrieved directly using the NCBI Entrez sequence retrieval tools. DNA sequence translations were performed using the program Transeq, a component of the EMBOSS package and provided by the EMBL-European Bioinformatics Institute Node (Rice et. al.; Trends Genet. 2000 June; 16(6):276-7). Domain architecture was defined with the aid of reading the literature and also by use of the SMART (Simple Modular Architecture Research Tool; EMBL) (Letunic et. al.; Nucleic Acids Res. 2002 Jan. 1; 30(1):2424). Default settings were used with the use of all bioinformatics tools, unless otherwise indicated in the text. At the time of the writing of this manuscript the above programs and Web interfaces could be accessed from the sites shown in Table 5.

TABLE 5
Resources/tools used for bioinformatics analyses

| Name | Site |
|---|---|
| Entrez Server | http://www.ncbi.nlm.nih.gov/Entrez/ |
| Blast Server | http://www.ncbi.nlm.nih.gov/blast/ |
| Compugen Bioccelerator | http://eta.embl-heidelberg.de:8000/misc/ |
| Server (EMBL) | |
| Compugen | http://eta.embl-heidelberg.de:8000/profw/ |
| PROFILEWEIGHT | |
| Emboss Transeq Server | http://www.ebi.ac.uk/emboss/transeq/ |
| SMART Server | http://smart.embl-heidelberg.de/ |
| ClustalX | ftp://ftp-igbmc.u-strasbg.fr/pub/ClustalX/ |

Typical members of the ErbB ligand family have already been described elsewhere (Harari et al., 1999; Harris et al., 2003; Strachan et al., 2001). Protein sequences for these ligands were extracted from the NCBI server by utilization of the Entrez sequence retrieval tool as well as by BLASTP searches against the NR protein database. Subsequently corresponding cDNA sequences were pulled out as reference links to the protein sequences, or by TBLASTN searches against the NR DNA database. Finally, genomic contigs encoding at least portions of the ErbB ligands were extracted by performing TBLASTN searches against the NCBI human and mouse genomic databases. Accession numbers of representative sequences are provided in Table 6. It should be noted, that these sequences are often redundantly represented in the database, and furthermore, there are the existence of alternative splice variants for some ligands. Thus the accession numbers given here are representative ones only. Reference to alternative accession numbers may be incorporated into the text.

TABLE 6
Accession numbers pertaining to genomic, transcript and
protein sequences encoding different ErbB-ligands

| | NCBI accession # | NCBI accession # | NCBI accession # |
|---|---|---|---|
| GENE | cDNA | Protein | Genomic Contig |
| NRG 1 Alpha | AF491780 * | AM71141.1 | NT_007995.10 |
| NRG1 Beta | AF491780 * | AAM71136.1 | |
| NRG2 Alpha | NP_004874 | NM_013982 | NT_029289 |
| NRG2 Beta | NM_013983 | NP_053586.1 | |
| NRG3 | XM_170640.1 | P56975 | NT_033890.2 |
| NRG4 | NM_138573.1 | NP_612640.1 | NT_024654.12 |
| EGF | NM_001963.2 | NP_001954.1 | NT_028147.9 |
| TGF alpha | K03222 | P01135 | NT_022184.9 |
| Amphiregulin | M30704 | AAA51781.1 | NT_006216.11 |
| HB-EGF | BC033097 | AAH33097.1 | NT_034777.1 |
| Betacellulin | S55606 | P35070 | NT_034698.1 |
| Epiregulin | NM_001432 | NP_001423.1 | NT_006216.11 |
| Epigen (Mouse) | AJ291391 | CAC39435.1 | NT_039307.1 |
| Epigen (Human) | | | NT_006216.1 |
| Lin-3 (C. elegans) | NM_171919 | NP_741490 | |
| Argos (Dros. melanogaster) | NM_079383 | NP_524107.2 | AE003527 |
| Argos (Musca domestica) | AF038405 | AAB92420 | |
| Argos (Dros virilis) | AB089249 | BAC56702 | |

* Numerous NRG1 variants are provided with this single accession.

It was initially decided to generate and analyze synthetic peptides encoding the EGF domains of class I variants of EGF, and NRG2 described in FIG. 4 (Sequence ID NOS:77 and 74). However, the synthetic peptides generated, were slightly shorter than that shown; spanning from five amino acids before the first cysteine residue to one amino acid residue carboxyl-to the fourth cysteine.

Generation of variant ErbB ligands devoid of loop C of the EGF domain.

It has previously been demonstrated that the EGF domain from different activatory ErbB ligands are both necessary and sufficient to confer receptor activation. For example, a refolded synthetic peptide harboring the NRG4 EGF domain alone is sufficient to elicit activation of ErbB4 (Harari et. al., 1999). Thus it was decided to synthetically generate and refold the EGF domain encoding two Class I variant ligands. Both truncated human EGF and truncated human NRG2 (of length 32 amino acids) were generated and refolded by air oxidation (described herein as EGF (1-32) and NRG2 (1-32). The peptides generated are a subsequence to peptide sequences listed in FIG. 4 (sequence IDs #77 and #74). In addition to human EGF, the sequence of mouse EGF (1-32) derived by a translated blast search against the mouse genome (tblastn search against the mouse genome, using the NCBI bast server), and the mouse EGF (1-32) was also synthesized and refoled in an independent manner, in this case by method of regioselective disulphide synthesis. The details of synthesis and refolding are given below:

### Synthesis and Refolding of Human EGF (1-32) and Human NRG2 (1-32)

Human EGF (1-32) i.e.: hEGF (1-32);
Sequence ID NO: 183 (Derived from SEQ ID NO: 77)

### NSDSECPLSHDGYCLHDGVCMYIEALDKYACK-OH

A first synthesis approach for HEGF (1-32) utilizing solid phase Fmoc technology starting from commercial available preloaded Tentagel-Lys(Boc)-Fmoc resin (Rapp Polymere, Germany) was not successful. One of the main problems during this synthesis was the high aggregation potential of the peptide sequence which led to incomplete couplings and sequence termination. In a second synthesis this problem was circumvented by switching to Boc chemistry. The reduced HEGF (1-32) peptide was thus synthesized with solid phase Boc technology utilizing preloaded Boc-Lys(2-Cl-Z)-Merrifield resin on a 1.5 mmol scale. Peptide sequence was synthesized using three equivalents of amino acids for coupling with DCCI. No recoupling and one acetylation after amino acid #11 (from N-terminal) was necessary. To minimize aspartimide formation Boc-Asp(OcHxl)-OH was used, for similar reason Boc-Glu(OcHxl)-OH was also employed. Cleavage from the resin with HF containing 10% anisole (v/v) yielded a crude peptide with moderate purity in HPLC and a dominant peak of a main product (t_{R} 37.9 mins, see HPLC #1). MS analysis of this crude showed the reduced form of the peptide (data not shown).

The bulk of the crude peptide was refolded by air-oxidation in water at pH 8-9 to produce the folded peptide: The reduced peptide was dissolved in water and the stirred solution was adjusted to pH 8.0 by addition of diluted aqueous NH₃ and solid NH₄Ac. Stirring was continued at room temperature and the reaction monitored by HPLC. Samples for analytical HPLC were acidified with acetic acid prior to injection and samples extracted at different time-points. HPLC analysis indicated that the refolding reaction was complete after 18 hours (data not) shown. A sample of the reaction after 18 hours subjected to the Ellman-Test shows no free thiols to be present.

Human NRG2 (1-32) i.e.: hNRG2 (1-32):
Sequence ID NO:184 (Derived from SEQ ID NO:74)

### GHARKCNETAKSYCVNGGVCYYIEGINQLSCK-OH

The reduced hNRG2 peptide was synthesized with solid phase Fmoc technology utilizing commercial available preloaded Tentagel-Lys(Boc)-Fmoc resin (Rapp Polymere, Germany). Peptide sequence was synthesized using two or three equivalents of amino acids for coupling with DIPCDI, beginning with coupling #14 (Fmoc-Val-OH)HOBt was additionally added to each coupling step. Recoupling was performed where necessary using TBTU/DIPEA with two equivalents of amino acid. Amino acids (from N-terminal) #2, 6, 13, 14, 15, 21, 24 27, 29, 31 were recoupled. Cleavage from the resin with King's cocktail yielded a crude peptide. MS analysis of this crude showed the presence of the reduced form of WPPL185 (data not shown). A reduction of disulfide-bridged oligomers with DDT did not result in an increased purity of reduced peptide.

A sample of the reduced peptide was dissolved in water and the stirred solution was adjusted to pH 8.5 by addition of diluted aqueous NH₃ and solid NH₄Ac. Stirring was continued at room temperature and the reaction monitored by HPLC. Samples for analytical HPLC were acidified with acetic acid prior to injection. Comparison of reaction samples after 2.5 hours and 21 hours indicated that the reaction was completed within a few hours. A sample of the reaction after 21 hours subjected to the Ellman-Test shows no free thiols to be present. Even prolonged reaction times did not have an influence on the efficiency or quality of disulfide-bridge forming. A sample of the reaction after 48 hours showed a by-product as a second new peak at t_{R} 29.2 Minutes. For this reason, for this experimental system, a reaction time of about 12-16 hours seems favourable.

### Mouse EGF (1-32):

### Sequence ID NO:185: (The Homologous Mouse Sequence to Human SEQ ID NO:183)

### NSYPGCPSSYDGYCLNGGVCMHIESLDSYTCK-OH

This peptide was synthesized using a regioselective disulphide synthesis protocol: The peptide was assembled on a 0.1 mmol scale by continuous flow Fmoc-solid phase synthesis as previously described (Dawson, et. al., (1999) J. Peptide Res. 53, 542-547). The solid support was Fmoc-Lys(Boc)-PAC-PEG-PS (PerSeptive Biosystems, USA), and a four-fold molar excess of HBTU-activated Fmoc-amino acids were used throughout. Na-Fmoc deprotection was with 20% piperidine in DMF. Amino acid side chain protection was afforded by the following: Asn and Gln, Trt; Asp and Glu, But; His, Trt; Tyr, But; Lys, Boc; Ser and Thr, But; and Cys(6,20), Trt; and Cys (14, 31), Acm. All derivatives were purchased from Auspep (Melbourne, Australia). No repeat amino acid couplings were carried out. At the end of assembly, cleavage from the solid supports and side chain deprotection was achieved by a 3.5-h treatment of the peptide-resin with trifluoroacetic acid (TFA) in the presence of phenol, thioanisole, ethanedithiol and water (82.5/5/5/2.5/5, v/v). An aliquot of the crude S-thiol (6, 20), S-Acm (14,31) peptide was purified by RP-HPLC on a Vydac C18 column using a gradient of acetonitrile containing 0.1% TFA. An aliquot of the purified peptide (50 Mg) was then subjected to disulfide bond formation between Cys 6 and 20 by treatment with 2-pyridyl disulfide in pH 8.5 buffer for 2 hours. It was subjected to preparative reversed-phase high performance liquid chromatography (RP-HPLC) on a Vydac C18 column (Hesperia, USA) using a 1%/min gradient of CH3CN in 0.1% aqueous TFA to yield 7.2 mgs. This was then subjected to formation of the second disulfide bond between Cys 14 and 31 by treatment with iodine in glacial acetic acid for 30 minutes at room temperature. The bis-disulfide mEGF(1-32) was HPLC-purified as before to give 2.5 mgs of highly homogeneous peptide that had the expected molecular mass as assessed by MALDI-TOF MS (described below).

### Mass Spectrometry Analysis of Peptides

### MILDI Analysis of Purified and Refolded Peptides:

Aqueous solutions of the synthetic peptides mEGF (1-32) hNRG2 (1-32) (1 mg/mL) were provided for analysis. 1.0 μL samples of each of these solutions were spotted onto a Perseptive Biosystems 10×10 MALDI target. A 10 mg/mL solution of α-cyano-4-hydroxycinnamic acid (Sigma-Aldrich Pty. Ltd, Sydney, Australia), which had been purified by recrystallisation from aq. ethanol, was prepared in 60% aq. Acetonitrile, 0.1% TFA immediately before use and 0.5 μL of this solution was added to each sample spot on the target. Samples were allowed to air dry at room temperature. TOF-MS data was acquired using a QSTAR Pulsar i mass spectrometer (Applied Biosystems, U.S.A.) equipped with an oMALDI II source. Ionisation was performed using a 337 nm wavelength nitrogen laser with a pulse rate of 20 Hz and a power level of 14.8 μJ. Data from [Glu¹]-fibrinopeptide B (Auspep Pty. Ltd, Melbourne, Australia) was used for TOF calibration. Mass accuracy in TOF-MS mode was better than 35 ppm. The theoretical monoisotopic molecular weights of the peptides were calculated using Protein Prospector (I) at the Asia-Pacific website (http://jpsl.ludwig.edu.au/). The molecular mass of refolded peptide HEGF (1-32) was determined independently on a different device, but by using a similar MALDI mass spectrometry approach. The results are summarized in Table 7.

TABLE 7
Mass Spectrometry measurements for refolded synthetic Class I Peptides.

| | | Mass reduction | | |
|---|---|---|---|---|
| | Reduced Peptide | with formation of | Oxidized Peptide | Oxidized Peptide |
| Sample | Expected Mass | two disulfide bridges | Expected mass | Observed Mass |
| hEGF (1-32) | 3579.5 | −4.0 | 3 575.47 | 3575.0 |
| mEGF (1-32) | 3463.4 | −4.0 | 3 459.38 | 3459.5 |
| hNRG2 (1-32) | 3508.6 | −4.0 | 3 504.59 | 3504.7 |

Monoisotopic mass measurements are given [M + 1]:

All refolded peptide products appeared to be reasonably pure as only a small number of peaks other than the MH+ reported were detected. The mass observed corresponds to the fully oxidized form of these peptides. Some minor deletion products were detected for hNRG2 (1-32), but their intensity was low relative to the major [M+1] reported. The mass observed corresponds to the fully oxidized form of these peptides.

### Determination of Disulfide Bridge Formation of Synthetic Peptides

It was initially assumed, from the structure determined from a number of ErbB ligand EGF domains, that (for the full length domain) that the encoded six cysteines form disulfide bridges in the following conformation: C1-C3; C2-C4; C5-C6 (Harari et al. 2000). Thus it was anticipated that the variant Class I peptides would form a C1-C3; C2-C4 conformation. In the case of mEGF (1-32), which was generated by a regioselective disulphide synthesis protocol, this expected order of disulfide bridging was directed by default during peptide synthesis. However, HEGF (1-32) and hNRG2 (1-32) were refolded by oxidation and the order of the disulfide bridge formation was not determined. Two approaches are performed to determine the disulfide bonding profile for these two ligands; proteolytic cleavage of the peptides, followed by mass spectrometry, and NMR determination.

Cleavage of the Peptides with the Protease V8.

HEGF (1-32) and hNRG2 (1-32) were suspended at a concentration of 1 mg/ml in 100 Mm bicarbonate buffer and then digested overnight at room temperature with lug V8 protease (Endoproteinase Glu-C; Roche Diagnostics GmbH), in order to produce cleavage of peptide bonds C-terminal of glutamic acid and aspartic acid residues. If fully digested, this cleavage pattern was to ideally result in the formation of peptide fragments between all the peptide bonds of hEGF (1-32). In the case of hNRG2 (1-32) however, cleavage with V8 produces fewer cuts, resulting in the generation of independent fragments harboring C1, C4 and Cys(2^{nd}+3^{rd} combined). The molecular mass of the tethered fragments were then measured, with the aim of determining the Cys-Cys bonding profiles for these air-oxidized peptides.

hEGF (1-32)

Cleavage with V8 resulted in the formation of novel bands of molecular weight [M+1] 1282.7 Da and 1522.72 Da, which closely resembles a disulfide bonding pattern of C1-C4 and C2-C3 (Table 8). A major peak of 3577.549 Da was also detected, which corresponds to the expected molecular mass of the full-length uncleaved hEGF+2 Da, which indicates that two hydrogen atoms have bound to this peptide after incubation in the V8 cleavage buffer. These data are consistent with the possibility that most of the peptide remained uncleaved after digest. Repeat digestion of hEGF (1-32) with V8 together with 10% acetonitrile failed to improve the yield of fully digested fragments (data not shown).

hNRG2 (1-32)

Incubation of NRG2 with V8 proteinase resulted in the formation of molecular masses consistent with a C1-C4 and C2-C3 disulfide bridge formation (Table 8). No evidence of other peaks corresponding to the formation of alternative disulfide bridge conformations were detected; nor was there evidence of uncleaved peptide. Thus to the resolution of detection, this experiment indicates that air oxidized hNRG2 (1-32) harbors a homogeneous structure in which C1-C4 and C2-C3, a result not originally anticipated.

### Interpretation of the Mass Spectrometry Results

The data provided here indicate the synthetic peptide hNRG2(1-32) and perhaps hEGF(1-32), after air oxidation, by the described method, have formed a disulfide bridge structure as follows: C1-C4; C2-C3, and is contrary to the expected bridge formation of C1-C3; C2-C4. If the interpretation of the mass spectrometry data is correct, then based on the disulfide bridge profile, the Class I variants may be folded in a different configuration to that expected by that extrapolated from known EGF domains structures (having six cysteines). Alternatively, it is technically possible that this uncleaved fraction may represent an alternatively folded population. It should be noted though that as it is assumed that a large fraction of hEGF(1-32) remained uncleaved after V8 digestion, To independently verify these findings, NMR analyses of the peptides are performed (see below).

TABLE 8
Predicted and measured masses of refolded
synthetic Class I peptides

| | Mass of | Possible | Predicted | |
|---|---|---|---|---|
| Fragment | Fragment | disulfide | Mass | Observed |
| harboring: | [M + H] | bridge | [M + H] | Mass |
| Cys-6 (C1) | 914.4267 | C1-C2 bridge | 3540.66 | ND |
| | | bound to C3-C4 | | ND |
| Cys-14 &Cys20 | 1769.7879 | C1-C3 bridge | 3540.66 | ND |
| (C2&C3)* | | bound to C2-C4 | | ND |
| Cys-31 (C4) | 862.4457 | C1-C4 bridged | 1773.8724 | 1774.09 |
| | | C2-C3 bridged | 1767.7879 | 1767.99 |
| | | Uncut | 3504.7 | ND |

hEGF (1-32)

| | Mass of | Possible | Predicted | Observed |
|---|---|---|---|---|
| Fragment | Fragment | disulfide | Mass | Mass |
| harboring: | [M + H] | bridge | [M + H] | [M + H] |
| Cys-6 (C1) | 671.28 | C1-C2 bridged | 1375.56 | ND |
| Cys-14 (C2) | 707.28 | C3-C4 bridged | 1423.67 | ND |
| Cys-20 (C3) | 814.35 | C1-C3 bridged | 1482.63 | ND |
| Cys-31 (C4) | 612.32 | C2-C4 bridged | 1316.6 | ND |
| | | C1-C4 bridged | 1280.6 | 1282.712 |
| | | C2-C3 bridged | 1518.63 | 1522.72 |
| | | Uncut | 3575.0 | 3577.549 |

These predictions and results are given in monoisotopic mass measurements [M + H].
Note:
ND—Not Detected. Predicted masses are adjusted to give a decrease in MW of 2 Da per disulfide bridge. Furthermore, all peptides retain a theoretical molecular mass of [M + 1], regardless of the number of fragments tethered together by disulfide bridges.
*As a result of the cleavage pattern of hNRG2 with V8 (1-32), C2 and C3 remain as a single fragment after cleavage. This will result in the inability to separate the fragments in two of the three possible permutations in which two pairs of Cys-Cys bonds are formed, as indicated above.

### Nuclear Magnetic Resonance (NMR) Spectral Analysis

The synthesized Class I ligands are being analysed by NMR. All 1H NMR spectra are recorded on a Bruker ARX 500 spectrometer equipped with a z-gradient unit. Peptide concentrations range from 1-3 mM. The ¹H NMR experiments include NOESY with a mixing time of 350 ms and TOCSY with a mixing time of 65 ms. All spectra are recorded at 303 K. Spectra are run over 6024 Hz with 4K data points, 400-600 FIDs, 16 (TOCSY) or 64 (NOESY) scans and a recycle delay of 1 s.

### Mitogenic Assay:

### Activatory Ligand Stimulated Mitogenesis

Before determining inhibitory activity of the class I variant ligands, it is important to first test if these ligands exhibit activatory mitogenic potential. BaF/3 cells transfected with the EGFR (BaF/3-EGFR; Walker et al, Growth Factors 16: 53-67, 1998) are washed three times to remove residual IL-3 and resuspended in RPMI 1640+10% FCS. Cells are then seeded into 96 well plates using a Biomek 2000 (Beckman) at 2×10⁴ cells per 200 microlitres and incubated for 4 h at 37 C in 10% CO2. To determine the efficacy of this system with a positive control, cells are first grown with titrating concentrations of activatory ligand EGF alone, to determine the minimum amount of ligand required to achieve maximal or sub-maximal receptor-mediated mitogenesis for these cells. EGF purified from mouse salivary glands (Burgess et al, Proc Natl Acad Sci USA. 79:5753-7 (1982)) at a concentration of approximately 200 μM, typically induce a sub-maximal to maximal mitogenic response in these cells. Titrating concentrations of ErbB variant ligands are added to the cells to test their mitogenic potential. In a similar manner, BaF/3 or different cells expressing a range of ErbB receptors, rendering them mitogenically responsive to ErbB ligand stimulation, are used to test these and other variant ligands for activatory ligand stimulated mitogenesis (exemplified in Harari et. al., 1999).

Preliminary results indicate that the ligands mEGF(1-32) and hNRG2(1-32) do not potentiate mitogenesis of the BaF/3-EGFR cells (data not shown).

### Inhibitory Mitogenic Assay

In serial dilutions, titrating concentrations of variant ErbB ligands are added to BaF/3-EGFR cells seeded into 96 well plates with duplicate±mouse EGF (typically within an order of magnitude of 200 μM). In one series of experiments, the variant ligands are pre-incubated with the BaF/3-EGFR cells for half an hour before mouse EGF is added. In an other series of experiments, the variant ligands are preincubated with mouse EGF, or other activatory ErbB ligands for half an hour before adding the mixture of ligands to the cells. Plates are incubated with 3H-Thymidine (1 microCi/well) for 18 hours prior to cell harvesting (Filtermate, Packard), cells being trapped onto Unifilter 96 GF/C plates (Packard). These plates are to dried for 1 hour before addition of Microscint 20 (Packard) scintillation cocktail (20 microlitre) to each well. 3H-Thymidine incorporation was determined using a TopCount NXT beta counter (Packard). In a similar manner, BaF/3 or different cells expressing a range of ErbB receptors, rendering them mitogenically responsive to ErbB ligand stimulation, are used to test these and other variant ligands for their ability to inhibit ligand-induced mitogenesis (exemplified in Harari et. al., 1999).

BIAcore™ Analysis of hNRG2 (1-32) & mEGF(1-32)-Receptor Binding Assays.

Biosensor analyses were performed using a BIAcore™ 3000. A CM-5 (research grade) sensor chip was immobilized with soluble EGFR (amino acids 1-501), soluble EGFR (amino acids 1-621) and soluble ErbB2 (amino acids 1-509) on flowcells 2, 3 and 4, respectively. Immobilizations were performed using amine coupling chemistry in 10 mM Sodium Acetate at pH 4.2. Varying concentrations (1.25 μM, 2.5 μM, 5 μM and 10 μM) of peptides were injected (30 μl) over the sensor surfaces in HBS running buffer (10 mM HEPES, 3.4 mM EDTA, 0.15M NaCl, 0.005% Tween 20, pH 7.4) at a flow rate of 5 μl/min. The surfaces were then regenerated by injecting 10 μl of 10 mM NaOH at a flow rate of 20 μl/min. The resulting sensor curves were subtracted against the blank channel (Flowcell 1) to yield the specific response.

BIAcore™ Analysis of hNRG2 (1-32) & mEGF(1-32)-Measuring Ligand-Ligand Interactions.

Biosensor analyses were performed using a BIAcore™ 3000. A CM-5 (research grade) sensor chip was immobilized with recombinant human or bovine EGF, TGF alpha and Betacellulin on flowcells 2, 3 and 4, respectively. Immobilizations were performed using amine coupling chemistry in 10 mM Sodium Acetate at pH 4.2. Varying concentrations (0.3 μM, 0.6 μM, 1.25 μM, 2.5 μM, 5 μM, 10 μM [and in some cases 50 μM]) of hNRG2(1-32), mEGF(1-32) & hEGF(1-32) were injected (30 μl) over the sensor surfaces in HBS running buffer (10 mM HEPES, 3.4 mM EDTA, 0.15M NaCl, 0.005% Tween 20, pH 7.4) at a flow rate of 5 μl/min. The surfaces were then regenerated by injecting 10 μl of 10 mM NaOH at a flow rate of 20 μl/min. The resulting sensor curves were subtracted against the blank channel (Flowcell 1) to yield the specific response.

### Biacore Results

### Class I ErbB Ligand Variants—ErbB Receptor Interactions:

Peptides hNRG2 (1-32) & mEGF(1-32) when added to a concentration of up to 10 uM, failed to demonstrate measurable binding to immobilized soluble ErbB1, and ErbB2 (data not shown).

### Class I ErbB Ligand Variants—ErbB Ligand Interactions

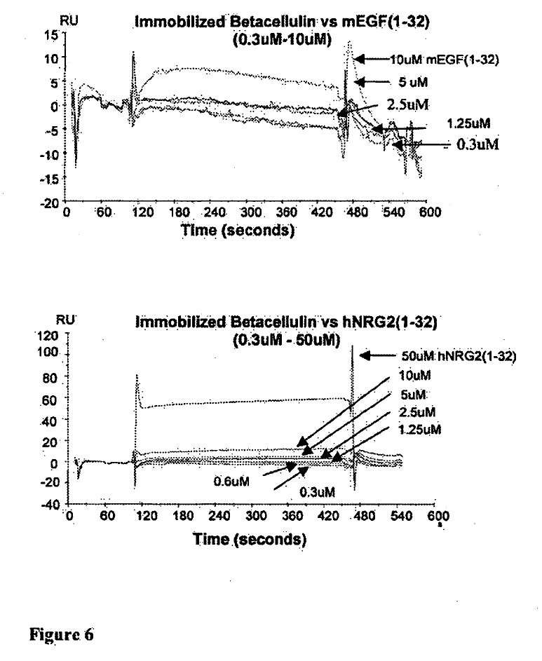

In an initial experiment, hNRG2 (1-32) and mEGF(1-32) and hEGF(1-32) were separately added to the immobilized agonist Betacellulin. For both hNRG2(1-32) and mEGF(1-32), weak binding to the Betacellulin was noted (FIG. 6). However, no binding of the hEGF(1-32) peptide to immobilized Betacellulin was detected (data not shown).

The present invention has been described with reference to specific preferred embodiments and examples. It will be appreciated by the skilled artisan that many possible alternatives will be apparent within the scope of the present invention which is not intended to be limited by the specific embodiments exemplified herein but rather by the following claims.

### REFERENCES

Altschul, S. F., Madden, T. L., Schaffer, A. A., Zhang, J., Zhang, Z., Miller, W. and Lipman, D. J. Gapped BLAST and PSI-BLAST: a new generation of protein database search programs. Nucleic Acids Res 25, 3389-402, 1997.
Barbacci, E. G.; Guarino, B. C.; Stroh, J. G.; Singleton, D. H.; Rosnack, K. J.; Moyer, J. D.; and Andrews, G. C. The structural basis for the specificity of epidermal growth factor and heregulin binding. J Biol Chem, 270(16):9585-9589, 1995.
Campion, S. R., and Niyogi, S. K. Interaction of epidermal growth factor with its receptor. Prog Nucleic Acid Res Mol Biol, 49:353-383, 1994.
Carpenter, G., and Cohen, S. Epidermal growth factor. J. Biol. Chem., 265:7709-7712, 1990.
Chema, R., Sugawara, H., Koike, T., Lopez, R., Gibson, T. J., Higgins, D. G. and Thompson, J. D. Multiple sequence alignment with the Clustal series of programs. Nucleic Acids Res 31, 3497-3500, 2003.
Crovello, C. S.; Lai, C.; Cantley, L. C.; and Carraway, K. L., 3rd. Differential signaling by the epidermal growth factor-like growth factors neuregulin-1 and neuregulin-2. J Biol Chem, 273(41):26954-26961, 1998.
Darnell, J.; Lodish, H.; and Baltimore, D., Molecular Cell Biology, Scientific American Books, USA (1986)
Defeo-Jones, D.; Tai, J. Y.; Vuocolo, G. A.; Wegrzyn, R. J.; Schofield, T. L.; Riemen, M. W.; and Oliff, A. Substitution of lysine for arginine at position 42 of human transforming growth factor-a eliminates biological activity without changing internal disulfide bonds. Mol. Cell. Biol., 9:4083-4086, 1989.
Engler, D. A.; Campion, S. R.; Hanser, M. R.; Cook, J. S.; and Niyogi, S. K. Critical functional requirements for the guanidinium group of the arginine 41 side chain of human epidermal growth factor as revealed by mutagenic inactivation and chemical reactivation. J. Biol. Chem., 267:2274-2281, 1992.
Falls, D. L. Neuregulins: functions, forms, and signaling strategies. Exp Cell Res., 284(1):14-30, 2003.
Groenen, L. C.; Nice, E. C.; and Burgess, A. W. Structure-function relationships for the EGF/TGF-a family of mitogens. Growth Factors, 11:235-257, 1994.
Harari, D.; Tzahar, E.; Romano, J.; Shelly, M.; Pierce, J. H.; Andrews, G. C.; and Yarden, Y. Neuregulin-4: a novel growth factor that acts through the ErbB4 receptor tyrosine kinase. Oncogene, 18(17):2681-2689, 1999.
Harari, D., and Yarden, Y. Molecular mechanisms underlying ErbB2/HER2 action in breast cancer. Oncogene, 19(53):6102-6114, 2000.
Harris, R. C.; Chung, E.; and Coffey, R. J. EGF receptor ligands. Experimental Cell Research, 284:2-13, 2003.
Howes, R.; Wasserman, J. D.; and Freeman, M. In vivo analysis of Argos structure-function. Sequence requirements for inhibition of the Drosophila epidermal growth factor receptor. Journal of Biological Chemistry, 273(7):4275-4281, 1998.
Jin, M. H.; Sawamoto, K.; Ito, M.; and Okano, H. The interaction between the Drosophila secreted protein argos and the epidermal growth factor receptor inhibits dimerization of the receptor and binding of secreted spitz to the receptor. Mol Cell Biol, 20(6):2098-2107, 2000.
Jones, J. T.; Akita, R. W.; and Sliwkowski, M. X. Binding specificities and affinities of egf domains for ErbB receptors. FEBS Lett, 447(2-3):227-231, 1999.
Jorissen, R. N.; Walker, F.; Pouliot, N.; Garrett, T. P.; Ward, C. W.; and Burgess, A. W. Epidermal growth factor receptor: mechanisms of activation and signalling. Exp Cell Res, 284(1):31-53, 2003.
Kurreck, J. Antisense technologies. Improvement through novel chemical modifications. Eur J Biochem, 270(8):1628-1644, 2003.
Maniatis, T.; Fritsch, E. F.; and Sambrook, J. Molecular Cloning: A Laboratory Manual. Cold Spring Harbor (New York: Cold Spring Harbor Laboratory), 1982.
Moghal, N., and Sternberg, P. W. The epidermal growth factor system in Caenorhabditis elegans. Exp Cell Res, 284(1):150-159, 2003.
Pennock, S.; and Wang, Z. Stimulation of Cell Proliferation by Endosomal Epidermal Growth Factor Receptor As Revealed through Two Distinct Phases of Signaling. Mol. Cell. Biol., 23(16): 5803-15, 2003
Sarup, J. C.; Johnson, R. M.; King, K. L.; Fendly, B. M.; Lipari, M. T.; Napier, M. A.; Ullrich, A.; and Shepard, H. M. Characterization of an anti-p185HER2 monoclonal antibody that stimulates receptor function and inhibits tumor cell growth. Growth Regul, 1(2):72-82, 1991.
Schnepp, B.; Donaldson, T.; Grumbling, G.; Ostrowski, S.; Schweitzer, R.; Shilo, B. Z.; and Simcox, A. EGF domain swap converts a drosophila EGF receptor activator into an inhibitor. Genes Dev, 12(7):908-913, 1998.
Shilo, B. Z. Signaling by the Drosophila epidermal growth factor receptor pathway during development. Exp Cell Res, 284(1):140-149, 2003.
Strachan, L.; Murison, J. G.; Prestidge, R. L.; Sleeman, M. A.; Watson, J. D.; and Kumble, K. D. Cloning and biological activity of epigen, a novel member of the epidermal growth factor superfamily. J Biol Chem, 276(21):18265-18271, 2001.
Summerfield, A. E.; Hudnall, A. K.; Lukas, T. J.; Guyer, C. A.; and Staros, J. V. Identification of residues of the epidermal growth factor receptor proximal to residue 45 of bound epidermal growth factor. J. Biol. Chem., 271:19656-19659, 1996.
Tzahar, E.; Moyer, J. D.; Waterman, H.; Barbacci, E. G.; Bao, J.; Levkowitz, G.; Shelly, M.; Strano, S.; Pinkas-Kramarski, R.; Pierce, J. H.; Andrews, G. C.; and Yarden, Y. Pathogenic poxviruses reveal viral strategies to exploit the ErbB signaling network. Embo J, 17(20):5948-5963, 1998.
Vinos, J., and Freeman, M. Evidence that Argos is an antagonistic ligand of the EGF receptor. Oncogene, 19(31):3560-3562, 2000.
Yarden, Y., and Sliwkowski, M. X. Untangling the ErbB signalling network. Nat Rev Mol Cell Biol, 2:127-137, 2001.

1. A polypeptide comprising a splice variant of an ErbB ligand encoded by differential exon usage comprising a truncated EGF domain devoid of the C-loop of the EGF domain.

2. The polypeptide according to claim 1 wherein the splice variant comprises a truncated ErbB receptor modulating EGF domain comprising only the first four of the six conserved cysteines found in an intact EGF domain.

3. The polypeptide of claim 2 wherein the fourth conserved cysteine of the truncated ErbB receptor modulating EGF domain is the penultimate amino acid at the C terminus of the polypeptide.

4. The polypeptide according to claim 3 having the sequence set forth in any one of SEQ ID NOS:73 to 84.

5. The polypeptide according to claim 3 having the sequence of any one of SEQ ID NOS: 93, 95-104, 109-110.

6. The polypeptide according to claim 2 wherein the splice variant comprises a receptor-modulating EGF domain having only the first four of the six conserved cysteines found in an intact EGF domain, further comprising an amino acid sequence encoded by an alternative exon other than the second exon encoding conserved cysteines five and six of the intact ErbB receptor-modulating EGF domain.

7. The polypeptide according to claim 6 having the sequence of any one of SEQ ID NOS:111-121.

8. The polypeptide according to claim 2 wherein the splice variant comprises a receptor modulating EGF domain having only the first four of the six conserved cysteines found in an intact EGF domain, wherein the splice variant has at least 90% homology to the aligned amino acid sequence of the same fragment in the EGF domain of a known ErbB ligand between cysteine 1 and cysteine 4.

9. The polypeptide of claim 8 wherein the splice variant has at least 95% homology to the aligned amino acid sequence of the same fragment in the EGF domain of a known ErbB ligand between cysteine 1 and cysteine 4.

10. The polypeptide of claim 1 wherein the N terminal flanking sequences preceding the cysteine 1 are at least 90% homologous to the same sequence in the EGF domain of a known ErbB ligand.

11. The polypeptide of claim 1 wherein the splice variant retains binding activity to at least one member of the ErbB/EGF receptor family.

12. The polypeptide of claim 10 which retains binding activity to the receptor cells with significantly reduced biological activity compared to an equimolar concentration of at least one known agonist ligand.

13. The polypeptide of claim 1 wherein the splice variant exerts inhibitory activity on at least one member of the ErbB/EGF receptor family.

14. The polypeptide of claim 10 which exerts inhibitory activity to the receptor when in a 100-fold molar excess or less, to at least one known agonist ligand.

15. An isolated polynucleotide encoding a splice variant of an ErbB ligand comprising a truncated ErbB-Receptor-modulating EGF domain devoid of the C-loop of the EGF domain.

16. The polynucleotide according to claim 15 wherein the splice variant comprises a truncated receptor-modulating EGF domain comprising only the first four of the six conserved cysteines found in an intact EGF domain.

17. The polynucleotide of claim 16 wherein the fourth conserved cysteine of the encoded truncated ErbB-Receptor modulating EGF domain is the penultimate amino acid at the C terminus of the polypeptide.

18. The polynucleotide according to claim 17 comprising the sequence of any one of SEQ ID NOS:128 to 139.

19. The polynucleotide according to claim 17 having the sequence of any one of SEQ ID NOS:148 to 165.

20. The polynucleotide according to claim 16 wherein the encoded splice variant comprises a receptor-modulating EGF domain having only the first four of the six conserved cysteines found in an intact EGF domain, further comprising an amino acid sequence encoded by an alternative exon other than the second exon encoding conserved cysteines five and six the of the intact ErbB receptor-modulating EGF domain.

21. The polynucleotide according to claim 20 having the sequence of any one of SEQ ID NOS:166-182.

22. The polynucleotide according to claim 16 wherein the splice variant comprises a receptor modulating EGF domain comprising only the first four of the six conserved cysteines found in an intact EGF domain, wherein the splice variant has at least 90% homology to the aligned amino acid sequence of the same fragment in the EGF domain of a known ErbB ligand between cysteine 1 and cysteine 4.

23. The polynucleotide of claim 22 wherein there is at least 95% homology to the aligned amino acid sequence of the same fragment in the EGF domain of a known ErbB ligand between cysteine 1 and cysteine 4.

24. The polynucleotide of claim 20 wherein the encoded N terminal flanking sequences preceding the cysteine 1 are at least 90% homologous to the same sequence in the EGF domain of a known ErbB ligand.

25. The polynucleotide of claim 15 wherein the splice variant exerts inhibitory activity to at least one member of the ErbB/EGF receptor family.

26. The polynucleotide of claim 25 which encodes a polypeptide that exerts inhibitory activity to the receptor on cells with significantly reduced biological activity compared to an equimolar amount at least one known agonist ligand.

27. An antisense oligonucleotide capable of specifically inhibiting the expression of a polypeptide according to claim 1.

28. A polynucleotide construct comprising an isolated polynucleotide encoding the splice variants claim 1.

29. A vector comprising the isolated polynucleotide encoding the splice variants of claim 1.

30. A host cell transformed with a polynucleotide encoding the splice variants of claim 1.

31. A host cell transformed with a polynucleotide according to claim 15.

32. A pharmaceutical composition comprising as an active ingredient a polypeptide according to claim 1.

33. A pharmaceutical composition comprising as an active ingredient a polynucleotide according to claim 15

34. A pharmaceutical composition comprising as an active ingredient an antisense oligonucleotide according to claim 27.

35. A method of treating a disease or disorder related to an ErbB receptor in an individual in need thereof comprising administering to the individual a therapeutically effective amount of a polypeptide comprising a splice variant of an ErbB ligand encoded by differential exon usage comprising a truncated EGF domain devoid of the C-loop of the EGF domain.

36. The method of claim 35 wherein the disease or disorder is selected from a neoplastic disease, a hyperproliferative disease, angiogenesis, restenosis, wound healing, psychiatric disorders, neurological disorders and neurological injuries.

37. A method of treating a disease related to pathological activity of at least one ErbB receptor comprising administering a therapeutically effective amount of a polynucleotide according to claim 15.

38. The method of claim 37 wherein the disease or disorder is selected from a neoplastic disease, a hyperproliferative disease, angiogenesis, restenosis, wound healing, psychiatric disorders, neurological disorders or neural injury.

39. A method for selectively enhancing or promoting the proliferation or differentiation of stem cells expressing ErbB receptors, comprising exposing the stem cells to an ErbB ligand splice variant, according to claim 1.

40. The method of claim 39 wherein the stem cells are of neural, cardiac or pancreatic lineages.
